(19) United States
(12) Patent Application Publication
(10) Pub. No.: US 2022/0267446 A1
(43) Pub. Date: Aug. 25, 2022

(54) COMBINATIONS OF ANTI-PD1 AND ANTI-CTLA4 ANTIBODIES

(71) Applicant: QILU PUGET SOUND BIOTHERAPEUTICS CORPORATION, Bothell, WA (US)

(72) Inventors: Zhi LIU, Shoreline, WA (US); William C. FANSLOW, III, Normandy Park, WA (US); Wei YAN, Samamish, WA (US); David L. TREIBER, North Bend, WA (US)

(21) Appl. No.: 17/676,036

(22) Filed: Feb. 18, 2022

Related U.S. Application Data
(60) Provisional application No. 63/151,030, filed on Feb. 18, 2021.

(51) Int. Cl. C07K 16/28; A61P 35/00

(52) U.S. Cl. CPC C07K 16/2818; A61P 35/00; A61K 48/00; C07K 2317/515; C07K 2317/51

(57) ABSTRACT

Provided herein are mixtures of antibodies comprising an anti-hCTLA4 antibody and an anti-hPD1 antibody, polynucleotides encoding such mixtures and host cells containing the polynucleotides, methods of making and using such mixtures, and pharmaceutical compositions comprising such a mixture of antibodies or (a) polynucleotide(s) encoding the mixture.

### CROSS-REFERENCE TO RELATED APPLICATIONS

This application claims the benefit of U.S. Provisional Application No. 63/151,030, filed on Feb. 18, 2021, the contents of which are hereby incorporated by reference in their entireties.

### FIELD

Described herein are compositions and methods within the field of therapeutic recombinant antibodies.

### SEQUENCE LISTING

The instant application contains a Sequence Listing which has been submitted electronically in ASCII format and is hereby incorporated by reference in its entirety. Said ASCII copy, created on Feb. 15, 2022, is named 126861-0003WO01_SL.txt and is 47,915 bytes in size.

### BACKGROUND

Monoclonal antibody technology has gradually become more mature and many successful monoclonal antibody products have been approved. However, the numbers of fully validated targets that are amenable for developing a single effective antibody drug have become increasingly scarce. It is often necessary to combine two or more individual antibodies to achieve full therapeutic benefits. For example, the combination of pertuzumab and trastuzumab can prolong the survival of breast cancer patients by more than 15 months, and the effect is far greater than that of trastuzumab monotherapy¹. Recently, the combination of atezolizumab and bevacizumab has been shown to significantly prolong the survival of metastatic hepatocellular carcinoma patients, highlighting the power of antibody combination therapy in bringing the synergy of immune checkpoint inhibitor and an angiogenesis inhibitor for the treatment of liver cancer².

The instant application contains a Sequence Listing which has been submitted electronically in ASCII format and is hereby incorporated by reference in its entirety. Said ASCII copy, created on Feb. 15, 2022, is named 126861-0003WO01_SL.txt and is 47,915 bytes in size.

Although many antibodies are currently being clinically evaluated in combinations, the regulatory path for the approval of antibody combination therapy is often long and costly. Typically, an antibody combination needs to be tested in a randomized trial against the individual antibodies alone, and quite often the individual antibodies should have been approved products before the antibody combination therapy can be approved. This process poses big challenges for a combination product of which each individual antibody has little efficacy by itself. Thus, the developers need to commit a lot of resources to develop single antibody beyond phase 1 trial before a combination study can be carried out. Because of this reason, development of a bispecific antibody, which is a single entity with simple regulatory path but can still engage two different targets, is often the option to achieve the same objectives of the antibody combinations. However, due to the limitation of its design, bispecific antibodies do not have the full flexibility of antibody combinations, particular in controlling the ratio of two antibody arms for each target. A single product with bispecific antibodies can only choose one type of Fc backbone whereas the antibody combination allows flexible selection of Fc backbone with an appropriate effect function and pharmacokinetics (PK) for each of the two antibodies. There is a pressing need for a new approach in developing antibody combination therapy.

Cytotoxic T-lymphocyte-associated antigen-4 (CTLA-4)³ and programmed cell death-1 (PD-1)⁴ are key immune checkpoint inhibitors (ICIs) of T cell immune response. CTLA-4 signaling limits the initiation of the T cell proliferation in the lymph nodes during the early phase of the immune response, whereas PD-1 restricts T cell activity later in the process in the tumor microenvironment⁵. CTLA-4 is critical for the function of regulatory T cells (Tregs), which are essential for suppressing autoimmunity and for maintaining self-tolerance but also play key roles in maintaining the suppressive tumor environment⁶. The CTLA-4 and PD-1 checkpoints are commonly exploited by tumors through the upregulation of the ligands for these inhibitory receptors on cancer cells or tumor infiltrating immune cells to evade and/or suppress the immune system^{7,8}. Blockade of CTLA-4 or PD-1 has resulted in dramatic reductions in the tumor burden in cancer patients, which has led to the regulatory approval of several products for multiple indications^{9,10} Moreover, PD-1 blocking antibodies such as nivolumab and CTLA-4 blocking antibodies such as ipilimumab have been shown to work through distinct but complementary mechanisms of actions ¹¹⁻¹³.

The combination of anti-PD-1 (aPD-1) and anti-CTLA-4 (aCTLA-4) antibodies have been tested extensively in multiple tumor types in clinical trials^{14,15} The main driver of the combination studies was to improve the overall response rates and the duration of the responses over PD-1 monotherapy, which typically works in about 20-30% of patients and in tumors with a high level of PD ligand 1 (PD-L1) expression. Addition of an anti-CTLA-4 antibody to PD-1 blockade increases the overall response rate numerically in almost all cases, which can often be translated into a longer duration of response and survival¹⁶. The combination of nivolumab and ipilimumab was approved for the treatment of melanoma, renal carcinoma, MSI-H CRC, NSCLC, MPM and hepatocarcinoma. However, the exact mechanisms of the increased responses for the combination are still unclear. In recently published multi-arm phase 3 studies, two different anti-CTLA-4 antibodies were tested in combination with an anti-PD-1 antibody or PD-L1 antibody for the first-line treatment of advanced non-small-cell Lung cancer (NSCLC)^{17,18.} A combination of ipilimumab, an IgG1 anti-CTLA-4 antibody, with nivolumab (an anti-PD-1 antibody) improves the overall survival compared to chemotherapy, and has been approved in the United States for patients with tumors expressing PD-L1≥1%¹⁷. In contrast, tremelimumab, an IgG2 anti-CTLA-4 antibody, when combined with durvalumab (an anti-PD-L1 antibody), does not improve progression-free or overall survival as compared to chemotherapy¹⁸. The difference in the outcomes between these two trials remains unexplained. Subgroup analysis indicates addition of ipilimumab can provide benefit over PD-1 monotherapy regardless of the PD-L1 level (>1% or <1%) or tumor mutation burden (TMB) threshold. Whereas tremelimumab can only provide benefits to patients in which the bTMB is higher than 20 mut/Mb. It is likely that there are multiple mechanisms in which anti-CTLA-4 antibodies provide benefits. Choosing the IgG1 isotype may be important for some of the CTLA-4 antibody activities in vivo. For example, although not confirmed in humans, multiple preclinical studies in mice have suggested the ability to deplete intra-tumor Tregs or alter the relative ratio of Treg to CD8 cells within tumor is critical to the anti-tumor response of an anti-CTLA-4 antibody. This ability depends on Fc mediated antibody effector functions¹⁹⁻²¹.

The combination PD-1 and CTLA-4 blockades can also lead to the increase of immune-related adverse events (irAE) compared to anti-PD-1 monotherapy²². Most common irAEs include pruritus, nausea, rash, diarrhea, and atony. Studies using different ratio of nivolumab and ipilimumab in the combination have demonstrated that the level of irAEs is more likely associated with the dose of ipilimumab than that of nivolumab. The current strategy to manage the elevated toxicity of the combination therapy is by reducing the dose and frequency of ipilimumab when administrated together with nivolumab²³. A commonly used regimen of 1 mg/kg ipilimumab every 6 weeks together with 3 mg/kg or 240 mg flat dose of nivolumab every 2 or 3 weeks can significantly reduce the dropout rate of patients due to serious AEs. However, the frequency of grade 3 or 4 AEs is still much higher with the combination therapy than with PD-1 monotherapy¹⁷. Interestingly in a recent study of quavonlimab, an anti-CTLA-4 IgG1 molecule, in combination with pembrolizumab (an anti-PD-1 antibody) in NSCLC patients, a low dose of CTLA-4 antibody (25 mg every 6 weeks) demonstrated an equal efficacy with a better safety profile than other higher dose regimens and was selected as the recommended phase 2 dose (RP2D) for further combination studies with a regular dose of pembrolizumab (200 mg every 3 weeks)²⁴. These findings provide a need for additional improvement on the safety and tolerability of the combination treatment.

As set forth above, further improvements in efficacy are needed. Moreover, in some studies such combination treatments have been associated with greater numbers of adverse events, including Grade 3 and 4 adverse events (see definitions below and Common Terminology Criteria for Adverse Events (CTCAE) version 5.0 2010, available at /ctep.cancer.gov/protocoldeyelopment/electronic_applications/docs/CTCAE_v5_Quick_Reference_8.5x11.pdf, which is incorporated herein by reference), than treatments using either an anti-PD1 or an anti-CTLA4 antibody alone. In addition, separate production two different antibodies is burdensome, expensive, and complex. Thus, there is a need in the art for more efficiently produced combinations of anti-PD1 and anti-CTLA4 antibodies and for combinations that are safer and more effective than existing combinations.

### SUMMARY OF INVENTION

Provided herein is a combination product that can deliver a dual blockade of PD-1 and CTLA-4 immune checkpoint pathways. In one embodiment, invention PSB205 (QL1706) is provided and was generated using a new technology platform that enables the production of two antibodies close to their natural forms from a single host cell line and is manufactured as a single product. The new PSB205 product is contemplated herein to maintain the enhanced anti-tumor activities of the dual-blockers but not induce increased incidence of irAEs. In contrast to a bispecific antibody, the anti-PD-1 and anti-CTLA-4 components of PS205 were individually designed to achieve the optimal target coverage and biological activities for each antibody as well as in the context of combination therapy. The anti-CTLA-4 component of PSB205 was engineered to have a faster clearance rate than other CTLA-4 antibodies, which leads to a reduced exposure within each treatment cycle. This unique profile of reduced anti-CTLA-4 exposure in the presence of steady duration of anti-PD-1 exposure is contemplated herein to improve tolerability and thus enable the patient to receive PSB205 for a longer period of time without discontinuation due to CTLA-4 antibody-mediated irAEs. Preliminary data from our phase 1 study showed that PSB205 (QL1607) was well tolerated and exhibited good anti-tumor responses in nasopharyngeal and lung cancer patients, including those resistant to PD-1 inhibitors.

Also provided herein are mixtures of antibodies comprising an anti-hCTLA4 antibody and an anti-hPD1 antibody, polynucleotides encoding such mixtures and host cells containing these polynucleotides, methods of making and using such mixtures and the polynucleotides encoding them, and pharmaceutical compositions comprising such a mixture of antibodies or (a) polynucleotide(s) encoding the mixture. The numbered items below describe aspects of these compositions and methods and are not meant to limit the scope of the description herein.

In a particular embodiment, PSB205 (QL1706) contains two engineered monoclonal antibodies (anti-PD-1 IgG4 and anti-CTLA-4 IgG1) that are expressed in a fixed ratio from the same single cell and manufactured together as one product (MabPair). To achieve optimal efficacy and safety profile, PSB205 was designed to bring different level of target coverage for PD-1 and CTLA-4 in a single product. In a particular embodiment, the anti-CTLA-4 antibody was engineered to have a shorter half-life to reduce its exposure and lower the risk of irAEs. In both preclinical experiment and phase 1 clinical trials, PSB205 has been found to demonstrate anti-tumor activities with evidence of functional dual blockade of both PD-1 and CTLA-4 pathways including an increase of KI67+CD8 T cells and ICOS+CD4 T cells. Preliminary data from phase I trials also showed that PSB205 (QL1607) was well tolerated with good anti-tumor effects in solid tumor patients, including those resistant to PD1 inhibitors.

The MabPair platform enables the delivery of antibody combination therapy in a single bifunctional product. MabPair molecules such as PSB205 (QL1706) can be specifically engineered to achieve the optimal level of target coverage for two different molecules such as anti-PD-1 and anti-CTLA-4 monoclonal antibodies, which can be translated into improved efficacy with good tolerability.

Also provided herein are particular embodiments set forth as numbered Aspects, such as for example as Aspect 1, corresponding to a mixture of antibodies comprising:
(a) an anti-human Programmed Death 1 (anti-hPD1) antibody comprising a heavy chain (HC) and a light chain (LC), wherein (1) the HC of the anti-hPD1 antibody is encoded by a nucleic acid sequence which encodes the amino acid sequence of SEQ ID NO: 1, and (2) the LC of the anti-hPD1 antibody is encoded by a nucleic acid sequence which encodes the amino acid sequence of SEQ ID NO: 5; and
(b) an anti-human cytotoxic T-lymphoctye associated protein 4 (anti-hCTLA4) antibody comprising an HC and an LC, wherein (1) the HC of the anti-hCTLA4 antibody is encoded by a nucleic acid sequence which encodes the amino acid sequence of SEQ ID NO: 13, and (2) the LC of the anti-hCTLA4 antibody is encoded by a nucleic acid sequence which encodes the amino acid sequence of SEQ ID NO: 17;
(c) wherein the weight for weight (w/w) ratio of the amount of the anti-hCTLA4 antibody in the mixture to the amount of the anti-hPD1 antibody in the mixture (anti-hCTLA4: anti-hPD1 ratio) ranges from 1:1 to 1:4,

wherein the anti-hPD1 antibody has an in vivo half-life (t_{1/2}) in a single dose study in cynomolgus monkeys of 220 to 380 hours and/or the anti-hPD1 antibody has an in vivo t_{1/2} of 135 to 300 hours when administered to a human who has not previously been dosed with the anti-hPD1 antibody, and
(d) wherein the anti-hCTLA4 antibody has an in vivo t_{1/2} in a single dose study in cynomolgus monkeys of 40 to 150 hours and/or the anti-hCTLA4 antibody has an in vivo t_{1/2} of 90 to 210 hours when administered to a human who has not previously been dosed with the anti-hCTLA4 antibody.

Aspect 2. The mixture of Aspect 1,

wherein the nucleic acid sequence which encodes the amino acid sequence of SEQ ID NO: 1 also encodes the amino acid sequence of SEQ ID NO: 10,
wherein the nucleic acid sequence which encodes the amino acid sequence of SEQ ID NO: 5 also encodes the amino acid sequence of SEQ ID NO: 12,
wherein the nucleic acid sequence which encodes the amino acid sequence of SEQ ID NO: 13 also encodes the amino acid sequence of SEQ ID NO: 22, and
wherein the nucleic acid sequence which encodes the amino acid sequence of SEQ ID NO: 17 also encodes the amino acid sequence of SEQ ID NO: 24.

Aspect 3. The mixture of Aspect 1 or 2, wherein the anti-hCTLA4:anti-hPD1 ratio ranges from 1:1 to 1:3.

Aspect 4. The mixture of Aspect 3, wherein the anti-hCTLA4:anti-hPD1 ratio ranges from 1:1.2 to 1:2.5.

Aspect 5. The mixture of Aspect 4, wherein the anti-hCTLA4:anti-hPD1 ratio ranges from 1:1.5 to 1:2.5.

Aspect 6. The mixture of Aspect 5, wherein the anti-hCTLA4:anti-hPD1 ratio ranges from 1:1.7 to 1:2.3.

Aspect 7. The mixture of any one of Aspects 1 to 6, wherein:

the amino acid sequences of the HC and LC of the anti-hPD1 antibody are encoded by the nucleic acid sequences of SEQ ID NOs: 2 and 6, respectively; and
the amino acid sequences of the HC and LC of the anti-hCTLA4 antibody are encoded by the nucleic acid sequences of SEQ ID NOs: 14 and 18, respectively.

Aspect 8. The mixture of any one of Aspects 1 to 7,

wherein the anti-hPD1 antibody has an in vivo t_{1/2} in a single dose study in cynomolgus monkeys of 250 to 350 hours and/or the anti-hPD1 antibody has an in vivo t_{1/2} of 140 to 250 hours when administered to a human who has not previously been dosed with the anti-hPD1 antibody, and
wherein the anti-hCTLA4 antibody has an in vivo t_{1/2} in a single dose study in cynomolgus monkeys of 70 to 130 hours and/or the anti-hCTLA4 antibody has an in vivo t_{1/2} of 90 to 140 hours when administered to a human who has not previously been dosed with the anti-hCTLA4 antibody.

Aspect 9. The mixture of any one of Aspects 1 to 8, wherein

when the mixture is administered to a group of at least ten human patients at a dose of no more than 5 mg/kg, no more than 15%, 14%, 13%, 12%, or 11% of the patients experience a grade 3 or grade 4 adverse event (AE).

Aspect 10. The mixture of Aspect 9, wherein

when the mixture is administered to a group of at least ten human patients at a dose of no more than 5 mg/kg, no more than 10%, 9%, or 8% of the patients experience a grade 3 or grade 4 AE.

Aspect 11. The mixture of Aspect 10, wherein

when the mixture is administered to a group of at least ten human patients at a dose of no more than 5 mg/kg, no more than 7%, 6%, or 5% of the patients experience a grade 3 or grade 4 AE.

Aspect 12. A pharmaceutical composition comprising the mixture of any one of Aspects 1 to 11.

Aspect 13. The pharmaceutical composition of Aspect 12, wherein the pH of the pharmaceutical composition is from pH 4.5 to pH 5.5.

Aspect 14. The pharmaceutical composition of Aspect 12 or 13, wherein the total protein concentration in the composition is from 20 mg/mL to 30 mg/mL.

Aspect 15. The pharmaceutical composition of any one of Aspects 12 to 14, wherein the pharmaceutical composition has an osmolality from 250 to 380 mOsm/kg.

Aspect 16. One or more polynucleotide(s) encoding the mixture of any one of Aspects 1 to 11.

Aspect 17. The polynucleotide(s) of Aspect 16, wherein the polynucleotide(s) comprise the nucleic acid sequences of SEQ ID NOs: 2, 6, 14, and 18.

Aspect 18. One or more vector(s) comprising the polynucleotide(s) of Aspect 16 or 17.

Aspect 19. The vector(s) of Aspect 18, which is (are) (a) viral vector(s).

Aspect 20. The vector(s) of Aspect 19, which is (are) (an) oncolytic viral vector(s).

Aspect 21. The vector(s) of Aspect 19 or 20, which is (are) (a) retroviral, adenoviral, adeno-associated viral (AAV), vaccinia viral, modified vaccina viral Ankara (MVA), herpes viral, lentiviral, measles viral, coxsackie viral, Newcastle Disease viral, reoviral, or poxviral vector(s).

Aspect 22. A host cell comprising the polynucleotide(s) of Aspect 16 or 17 and/or the vector(s) of Aspect 18, wherein the host cell can produce the mixture of any one of Aspects 1 to 11.

Aspect 23. The host cell of Aspect 22, wherein the anti-hCTLA4:anti-hPD1 ratio of the mixture produced by the host cell ranges from 1:1.2 to 1:3.

Aspect 24. The host cell of Aspect 23, wherein the anti-hCTLA4:anti-hPD1 ratio of the mixture produced by the host cell ranges from 1:1.5 to 1:2.5.

Aspect 25. The host cell of Aspect 24, wherein the anti-hCTLA4:anti-hPD1 ratio of the mixture produced by the host cell ranges from 1:1.7 to 1:2.3.

Aspect 26. The host cell of any one of Aspects 22 to 25, which is a CHO cell or a mouse myeloma cell.

Aspect 27. A method for making a mixture of antibodies comprising the following steps: culturing the host cell of any one of Aspects 22 to 26; and recovering the mixture of antibodies from the culture supernatant or the host cell mass.

Aspect 28. A method for treating a patient having a cancer, an immunodeficiency disorder, or an infection comprising:

(a) administering to the patient a dose of the mixture of any one of Aspects 1 to 11 or the pharmaceutical composition of any one of Aspects 12 to 15 to the patient, or
(b) administering to the patient a dose of the polynucleotide(s) of Aspect 16 or 17 or the vector(s) of any one of Aspects 18 to 21.

Aspect 29. The method of Aspect 28(a), wherein the dose of the mixture or pharmaceutical composition is administered about twice a week, once a week, or once every two, three, four, five, six, seven, or eight weeks, and wherein the dose of the mixture or pharmaceutical composition is described by one or more of the following:

(1) the dose is at least about 0.5, 1.0, 2.0, 3.0, 4.0, 5.0, 6.0, 7.0, or 8.0 mg/kg;
(2) the dose is at most about 9, 8, 7, 6, 5, 4, or 3 mg/kg;
(3) the dose is about 1.0, 2.0, 3.0, 4.0, 5.0, 6.0, 7.0, or 8.0 mg/kg;
(4) the dose is about 200, 225, 250, 275, 300, 325, 350, 375, 400, 425, 450, 475, or 500 mg;
(5) the dose is at least about 75, 100, 125, 150, 200, 225, or 250 mg; and
(6) the dose is at most about 600, 500, 400, or 300 mg.

Aspect 30. The method of Aspect 29, wherein

the dose is at least 3 mg/kg and no more than 5 mg/kg, and/or

the dose is at least 180 mg and no more than 400 mg,

wherein the dose is administered about once every three weeks.

Aspect 31. The method of Aspect 30, wherein the dose is about 5 mg/kg.

Aspect 32. The method of Aspect 30, wherein the dose is 300 to 400 mg.

Aspect 33. The method of any one of Aspects 29 to 32,

wherein the patient has a cancer,
wherein the mixture or the pharmaceutical composition is administered to at least 10 patients, and
wherein the objective response rate (ORR) is at least 5, 10, 15, 20, 25, 30, or 35 percent and/or the disease control rate (DCR) is at least 25, 30, 35, 40, 45, 50, 55, or 60 percent.

Aspect 34. The method of Aspect 28(b), wherein the dose of the polynucleotide(s) or the vector(s) is administered about twice a week, once a week, or once every two, three, four, five, six, seven, or eight weeks, and wherein the dose of the polynucleotide(s) or vector(s) is described by one or more of the following:

(1) the dose is at least about 5×10⁹ copies of the polynucleotide(s) or the vector(s) per kilogram of patient body weight (copies/kg);
(2) the dose is at most about 10¹⁵ copies/kg;
(3) the dose is from about 10¹⁰ copies/kg to about 10¹⁴ copies/kg; and
(4) the dose is about 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, or 10¹⁵ copies.

Aspect 35. The method of any one of Aspects 28 to 34, wherein the dose of the mixture, pharmaceutical composition, polynucleotide(s), or vector(s) is administered by intravenous injection, including infusion or bolus injection, subcutaneous injection, or intramuscular injection.

Aspect 36. The method of any one of Aspects 28 to 35, wherein the patient has melanoma, lung cancer, including squamous non-small cell lung cancer and small cell lung cancer, nasopharyngeal cancer, squamous cell carcinoma of the head and neck, gastric or gastroesophageal carcinoma, clear cell or non-clear cell renal cell carcinoma, urothelial cancer, soft tissue or bone sarcoma, mesothelioma, classical Hodgkin lymphoma, primary mediastinal large B-cell lymphoma, bladder cancer, Merkel cell carcinoma, neuroendocrine carcinoma, cervical cancer, hepatocellular carcinoma, ovarian cancer, or microsatellite instability high (MSI-H) or DNA mismatch repair deficient (dMMR) adult and pediatric solid tumors.

Aspect 37. The method of any one of Aspects 28 to 36, wherein the patient is treated with a chemotherapeutic agent or radiation before, after, or concurrently with the dose of the mixture, the pharmaceutical composition, the polynucleotide(s), or the vector(s).

Aspect 38. The method of any one of Aspects 28 to 37, wherein the dose of the mixture, the pharmaceutical composition, the polynucleotide(s), or the vector(s) is administered to at least ten patients, wherein the patients to whom the dose has been administered are not treated concurrently with radiation or with a chemotherapeutic agent, and wherein no more than 15%, 14%, 13%, 12%, or 11% of the patients to whom the dose has been administered experience a grade 3 or grade 4 AE.

Aspect 39. The method of Aspect 38, wherein no more than 10%, 9%, or 8% of the patients to whom the dose has been administered experience a grade 3 or grade 4 AE.

Aspect 40. The method of Aspect 39, wherein no more than 7%, 6%, 5%, 4%, 3%, 2%, 1%, or 0% of the patients to whom the dose has been administered experience a grade 3 or grade 4 AE.

### BRIEF DESCRIPTION OF THE FIGURES

FIG. 1: Plasmid map for vector encoding anti-hPD1 IgG4 antibody PSB103. Various genetic elements in the plasmid are labeled as follows: Pro PGK, promoter of phosphoglycerate kinase; DHFR, dihydrofolate reductase gene; SV40 pA, SV40 polyadenylation signal; Pro EF2/CMV, hybrid promoter of elongation factor 2 and cytomegalovirus (CMV); anti-PD-1 IgG4-HC, sequence encoding the PSB103 anti-hPD1 HC; CMV pA, polyadenylation signal from CMV; Pro CMV/EF1, hybrid promoter of CMV and elongation factor 1; anti-PD-1 LC, sequence encoding the LC of the PSB103 anti-hPD1 antibody, which is a kappa LC; Puro Resis, puromycin resistance gene; pMB1 ori, pMB1 origin of DNA replication; Kana Resis, kanamycin resistance gene; NruI, recognition site of restriction enzyme NruI.

FIG. 2: Selection scheme for obtaining a CHO cell line expressing PSB103. This process is explained in Example 1. The box labeled “Transfection” at left represents the transfection of CHO cells with the plasmid diagrammed in FIG. 1. Transfected cells were split into two pools, which were subjected to two different phase 1 selection regimens, as shown in the two middle boxes. Then the two phase 1 pools were each split into two pools, which were subjected to two different phase 2 selection regimens, as shown in the four boxes at right.

FIG. 3: Plasmid map for vector encoding the HC of anti-hCTLA4 antibody PSB105. Various genetic elements in the plasmid are labeled as follows: Promoter PGK Mm, promoter of phosphoglycerate kinase from Mus musculus; Promoter EF1a Hs, promoter of elongation factor 1-alpha from Homo sapiens; Intron EF1a Hs, intron of elongation factor 1-alpha from H. sapiens; anti-CTLA-4 IgG1-HC, DNA encoding the IgG1 HC of anti-hCTLA4 antibody PSB105; EES, expression enhancement sequence proprietary to Atum (Newark, Calif.); HPRE, hepatitis B-virus post-transcriptional regulatory element; Ocrabbit, the polyadenylation (poly(A)) signal of the rabbit beta-globin gene; HS4 Insulator, HS4 insulator element from the chicken beta-globin gene; Ori pUC, origin of DNA replication for replication in Escherichia coli; Kanamycin Resistance, kanamycin resistance gene; NruI, recognition site for restriction enzyme NruI; pA Globin Hs, poly(A) signal of H. sapiens beta-globin gene; and Hygromycin Resistance, hygromycin resistance gene.

FIG. 4: Plasmid map for vector encoding the LC of anti-hCTLA4 antibody PSB105. Various genetic elements in the plasmid are labeled as follows: P-EF1a Hs, promoter of elongation factor 1-alpha from H. sapiens; EF1-Hs Exon 1, exon 1 of elongation factor-alpha from H. sapiens; Intron EF1a Hs, intron of elongation factor 1-alpha from H. sapiens; Intron acceptor Mm IgH, the IgH intron acceptor from Mus musculus; anti-CTLA-4 LC, DNA encoding the LC of anti-hCTLA4 antibody PSB105; EES, expression enhancement sequence proprietary to Atum (Newark, Calif.); HPRE, hepatitis B-virus post-transcriptional regulatory element; Ocrabbit, the poly(A) signal of the rabbit beta-globin gene; HS4 Insulator, HS4 insulator element from the chicken beta-globin gene; Ori pUC, origin of DNA replication for replication in E. coli; Kanamycin Resistance, kanamycin resistance gene; NruI, recognition site for restriction enzyme NruI; pA Globin Hs, poly(A) signal of H. sapiens beta-globin gene; Mm Glutamine Synthetase, glutamine synthetase from M. musculus; and P-PGK Mm, promoter of phosphoglycerate kinase from M musculus.

FIGS. 5A-B: Binding specificity of PSB103 and PSB105. Experiments are described in Example 2. Panels FIG. 5A and FIG. 5B, respectively, show results for the anti-hPD1 antibody PSB103 and the anti-hCTLA4 antibody PSB105. In both panels A and B, the ligands tested for binding to these antibodies are indicated below the x axes from left to right as follows: huPD1, the extracellular domain of human PD1 fused to an Fc fragment; muPD1, the extracellular domain of murine PD1 fused to a histidine-avi tag (which enables the efficient purification (histidine tag) of the protein and labeling of the protein (avi tag) with biotin); huPDL1, the extracellular region of human PDL1 fused to a histidine-avi tag; huCD28, the extracellular domain of human CD28 fused to an Fc fragment; and huCTLA4, the extracellular domain of human CTLA4 fused to an Fc fragment. Bars with diagonal stripes, discontinuous short horizontal lines, or heavy continuous horizontal lines indicate data from samples containing, respectively, 100 ng/mL, 33 ng/mL, or 0 ng/mL of the tested ligand. The y axes show optical density at 450 nm (OD₄₅₀), which reflects the amount of binding in this assay.

FIGS. 6A-B: Single-dose pharmacokinetics of PSB103 and an unrelated IgG4 antibody in cynomolgus monkeys (Macaca fascicularis). This experiment is described in Example 3. Panels FIG. 6A and FIG. 6B show, respectively, data from monkeys injected with PSB103 and the unrelated IgG4 antibody. The x and y axes show, respectively, the time (hours) after the injection of the test antibody and the concentration of antibody detected in serum (μg/mL). Symbols signify as follows: open and filled circles indicate data from a first and second aliquot, respectively, both from a sample from a female monkey; and open and filled triangles indicate data from a first and second aliquot, respectively, both from a sample from a male monkey.

FIG. 7: Single-dose pharmacokinetics of PSB105, an unrelated IgG1 antibody, and ipilimumab in cynomolgus monkeys. This experiment is described in Example 3. The x and y axes show, respectively, the time (hours) after the injection of the test antibody and the amount of antibody detected in serum (μg/mL). Symbols indicate as follows: closed and open squares indicate data from male and female monkeys, respectively, dosed with PSB105; closed and open circles indicate data from male and female monkeys, respectively, dosed with the unrelated IgG1 antibody; and closed and open triangles indicate data from male and female monkeys, respectively, dosed with ipilimumab.

FIGS. 8A-B: General selection scheme for producing host cells expressing both PSB103 (an anti-hPD1 antibody) and PSB105 (an anti-hCTLA4 antibody). Panel FIG. 8A diagrams the creation of host cells expressing the anti-hPD1 antibody PSB103, which is described in Example 1. The “Y” symbol inside the cells represents antibody. Panel FIG. 8B diagrams the creation of cells expressing both PSB103 and the anti-hCTLA4 antibody PSB105, which is described in Example 4.

FIG. 9: Diagram of drug selection protocol for selecting cells expressing PSB103 and PSB105. This process is described in Example 4. The box at far left represents the transfection of G19G4-4B4 cells (expressing PSB103) with vectors encoding the heavy and light chains of PSB105. After 48 hours this culture was subdivided into three cultures, which were subjected to different drug selections (indicated by the three boxes in the middle of FIG. 9). These three cultures were seeded into 96 well plates (indicated by the nine boxes at the right of FIG. 9). As indicated, 48 of these wells exhibiting growth under selection were expanded in 12-well microtiter plates and assayed for the relative amounts of anti-hPD1 and anti-hCTLA4 antibody they produced.

FIGS. 10A-C Fluorescence activated cell sorting (FACS) analysis of transfected cells. As explained in Example 4, cell lines transfected with vectors encoding both PSB103 and PSB105 were screened using FACS to find lines where most individual cells in the cell line expressed both PSB103 (an IgG4 anti-hPD1 antibody) and PSB105 (an IgG1 anti-hCTLA4 antibody). Panel FIG. 10A shows the portions of a graph of FACS data where cells expressing only PSB103, only PSB105, or both would be expected to appear. As indicated, the anti-IgG1 antibody used to detect PSB105 was labeled with fluorescein isothiocyanate (FITC), and the anti-IgG4 antibody used to detect PSB103 was labeled with allophycocyanin (APC). Panel FIG. 10B shows data from a cell line where most individual cells in the cell line expressed both PSB103 and PSB105 and very few expressed only PSB103 or PSB105. Panel FIG. 10C shows data from a cell line where clearly detectable numbers of cells express only PSB103 or PSB105, while the majority of individual cells express both.

FIGS. 11A-B: Screen of clonal cell lines for total antibody titer and percent anti-hPD1 antibody. As explained in Example 4, total antibody titer (shown in panel FIG. 11A) and percent of the antibody that was the anti-hPD1 antibody PSB103 (shown in panel FIG. 12B) was determined. As indicated the clonal cell lines are identified by clone number under the x axes, followed in parenthesis by the number of days the cells had been cultured when the antibody was harvested.

FIGS. 12A-C: Productivity and growth characteristics of clonal cell line 20F5. Methods are explained in Example 4. In panel FIG. 12A, the x axis indicates the population doubling level (PDL, i.e., the number of cell doublings post thaw from a research cell bank (RCB)) of the culture used to initiate the fed-batch production culture (from which the data in panels FIG. 12A and FIG. 12B comes), which is indicated by a number below each bar. The presence or absence of hygromycin B (HGB) and methotrexate (MTX) in the medium of the cultures used to initiate the fed batch cultures is indicated below the PDLs. All fed-batch cultures were in medium lacking MTX and HGB (−MTX/−HGB medium). The cultures represented by the four leftmost bars were in medium lacking MTX and HGB (−MTX/−HGB) for nine to ten cell doublings prior to initiation of fed batch cultures, which were also in −MTX/−HGB medium. Prior to that, these were in medium containing MTX and lacking HGB (+MTX/−HGB medium). Thus, the culture represented by the leftmost bar was in −MTX/−HGB medium for the whole of its propagation to a PDL of 9.2, when a fed batch culture initiated from this culture. The cultures represented by the fifth and sixth bars from the left were in +MTX/−HGB and +MTX/+HGB medium, respectively, for the number of cell doublings indicated by their PDLs. Fed batch cultures (from which the data in panels FIG. 12A and FIG. 12B comes) were initiated from these cultures in −MTX/−HGB medium. The y axis indicates total antibody productivity (grams/liter) of the fed-batch culture, which was determined from samples taken 11 days after the initiation of each fed batch culture (Day 11). In panel FIG. 12B, individual bars in each of the three groups of bars represent data from the same fed batch cultures in the same order as in panel FIG. 12A. The fed batch culture day at which the analyzed sample was taken is indicated under each of the three groups of bars below the x axis. The y axis indicates the percent of the total antibody produced that is the anti-hPD1 antibody, i.e., PSB103. Panel FIG. 12C indicates the cell doubling time of cell line 20F5 as a function of PDL in +MTX/+HGB medium (dashed line) or in +MTX/−HGB medium (solid line). As indicated, the x axis indicates PDL, and the y axis indicates cell doubling time.

FIGS. 13A-C: Analysis of PSB103 and PSB105 isolated from a preparation of PSB205 (called PSB103-S and PSB105-S) by liquid chromatography-mass spectrometry (LC-MS). This experiment is described in Example 5. Panels FIG. 13A, FIG. 13B, and FIG. 13C show, respectively, data from PSB103-S, PSB105-S, and the preparation of PSB205 from which these antibodies were isolated. Sizes of peaks in daltons are indicated near the largest peaks. Glycosylation states of the HC residue N297 (numbered according to the numbering scheme of Edelmann et al. (1969), The covalent structure of an entire γG immunoglobulin molecule, Proc. Natl. Acad. Sci. USA 63: 78-85, which is incorporated herein by reference; N297 corresponds to positions N296 and N298 in SEQ ID NOs: 1 and 13, respectively) of various species are indicated by the following markings: GOF/GOF, a glycan including three mannose (Man3) residues, four N-acetyl glucosamine residues ((glcNAc)4), and one fucose residue ((Fuc)1) (Man3(glcNAc)4(Fuc)1) on the N297 of each HC; GOF/GOF-GlcNAc, a Man3(glcNAc)4(Fuc)1 glycan on the N297 of one HC and a Man3(glcNAc)3(Fuc)1 glycan on the N297 of the other HC; and GOF/G1F, a Man3(glcNAc)4(Fuc)1 glycan on the N297 of one HC and a glycan including one galactose residue ((Gal)1), three mannose residues, four N-acetyl glucosamine residues, and one fucose residue ((Gal)1Man3(glcNAc)4(Fuc)1) on the N297 of the other HC. For depictions of these chemical structures see, e.g., Yang et al. (2016), Ultrafast and high-throughput N-glycan analysis for monoclonal antibodies, MAbs 8(4): 706-717 and Symbol nomenclature for glycans (SNFG), available at https://www.ncbi.hlm.nih.gov/glycans/sngf.html, which is incorporated herein by reference. The x axes show masses (in Daltons) of antibody species detected, and the y axes show the percent intensity which reflects the percent abundance in the sample analyzed.

FIG. 14A-B: Analysis of different lots of PSB205 by LC-MS. Two different lots of PSB205 were analyzed by LC-MS as described in Example 5. Panels FIG. 14A and FIG. 14B show data from the PSB205-Tox lot and the PSB205-GMP lot, respectively. The x axes show masses (in Daltons) of antibody species detected, and the y axes show the percent intensity which reflects the percent abundance in the sample analyzed. Glycosylation states of the antibodies are indicated as in FIG. 13.

FIG. 15: Heat capacity plot from a GMP lot of PSB205. This experiment is described Example 7. The x axis indicates temperature (° C.), and the y axis indicates molar heat capacity (kcal/mole/° C.).

FIGS. 16A-D: Flow cytometric analysis of cells stimulated with a CMV-infected cell lysate and an antibody and labeled with an anti-CD8 antibody and an HLA-CMV dextramer. This experiment is described in Example 8. The x axes of all panels show fluorescence from the FITC-labeled anti-CD8 antibody, and the y axes show fluorescence from the phycoerythrin (PE)-labeled HLA-CMV dextramer. The boxed area in each panel indicates the CD8⁺CMV⁺ cells, and the number to the left of the boxed area indicates the percentage of all cells that are CD8⁺CMV⁺ cells. Panel FIG. 16A shows data from cells stimulated with the IgG1 isotype control antibody and the CMV-infected cell lysate. Panel FIG. 16B shows data from cells stimulated with PSB103 and the CMV-infected cell lysate. Panel FIG. 16C shows data from cells stimulated with PSB105 and the CMV-infected cell lysate. Panel FIG. 16D shows data from cells stimulated with PSB205 and the CMV-infected cell lysate.

FIGS. 17A-B: Quantitation of percentage of and absolute numbers of CD8⁺CMV⁺ cells among cells stimulated with a CMV-infected cell lysate and an antibody. This experiment is described in Example 8. Panel FIG. 17A shows the percentage of all cells that were CD8⁺CMV⁺ cells. The x axis indicates the antibodies used to stimulate the samples, and the y axis shows the percentage of all cells that were CD8⁺CMV⁺ cells. The graph in panel FIG. 17B shows the absolute numbers of CD8⁺CMV⁺ cells detected on its y axis and the antibodies used to stimulate the sample on its x axis. In both panels, the error bars indicate standard deviation. For simplicity, only half of the error bar is shown.

FIG. 18: Effect of PSB205 versus its component antibodies in a tumor model system. Experiment is described in detail in Example 9. The x axis shows days in the course of the experiment, and the y axis shows the tumor volume in mm³. Tested antibody treatments for the tumors are indicated by symbols as follows: filled circles, IgG negative control antibody; filled squares, PSB103 (anti-hPD1 antibody); filled upward-pointing triangles, PSB105 (anti-hCTLA4 antibody); and filled downward-pointing triangles, PSB205 (a mixture of PSB103 and PSB105). The asterisks indicate the following p values for the level of statistical significance of the difference between data for the IgG control antibody and data for PSB205: *, p=0.03; **, p=0.01; and ***, p=0.0006.

FIG. 19: Change from baseline in tumor diameter in individual patients. Methods are described in Example 10. The bars show the change from baseline in tumor diameter for each individual human patient among 32 of the evaluable patients listed in Table 12, including 13 lung cancer (LC) patients (blue bars) and 19 nasopharyngeal cancer (NPC) patients (brown bars). The y axis indicates the change in tumor diameter in mm. The change in diameter is also indicated above each bar (for increases) or below each bar (for decreases). The dosage of PSB205 administered to each patient is indicated directly below the x axis. The three bars marked by stars above them indicate patients that had new tumors in addition to having a change in the diameter of their original tumor.

FIGS. 20A-F: Generation and characterization of PSB205. (FIG. 20A): Principle of MabPair technology for producing two correctly assembled antibodies from a single mammalian cell line. Uniquely designed HC pairing keys and HC/LC pairing keys can be used to control the cognate HC and LC pairing and eliminate undesirable byproducts. (FIG. 20B): Co-expression of PSB103 and PSB105 in the production cell line was detected by intracellular staining of hu IgG4 and hu IgG1 specific reagents respectively. (FIG. 20C): PSB205 size variants were analyzed by size-exclusion HPLC. The chromatogram shows the main peak for monomers of the two mAbs overlaid, frontal minor peak(s) for high molecular weight (HMW) species, and post minor peak(s) for low molecular weight (LMW) species (not detected) in PSB205. As a result, the PSB205 purity as defined by the monomers (the main peak) was typically measured as 97-99% for different batches. (FIG. 20D) Baseline separation of the two mAbs in PSB205 was achieved by the hydrophobic interaction HPLC method. Thus it served as a tool to determine the concentration ratio of the two mAbs, [anti-PD-1]: [anti-CTLA-4] (w/w).

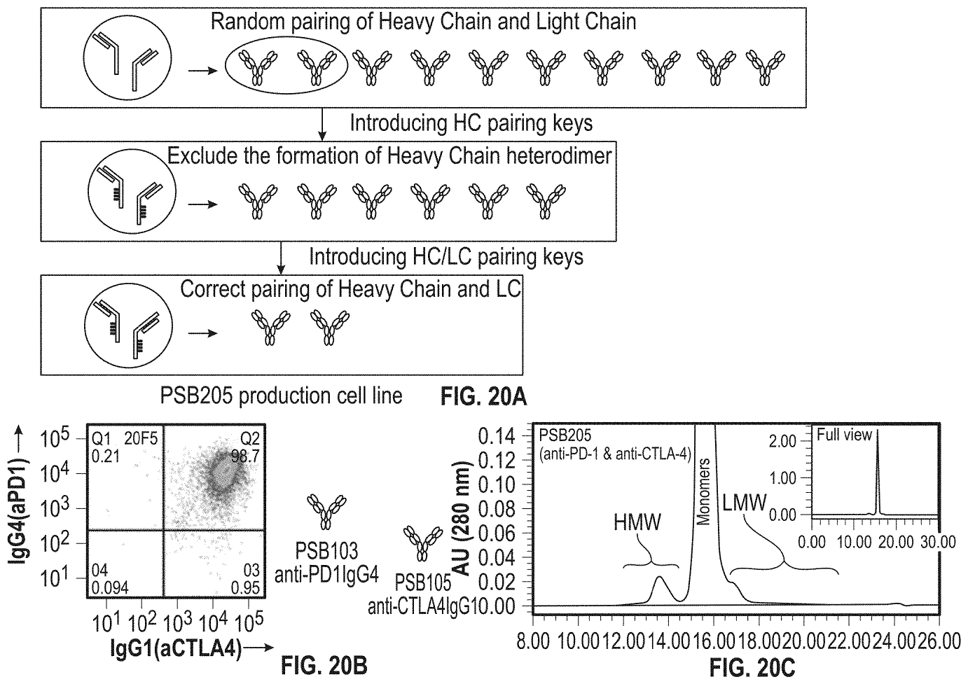

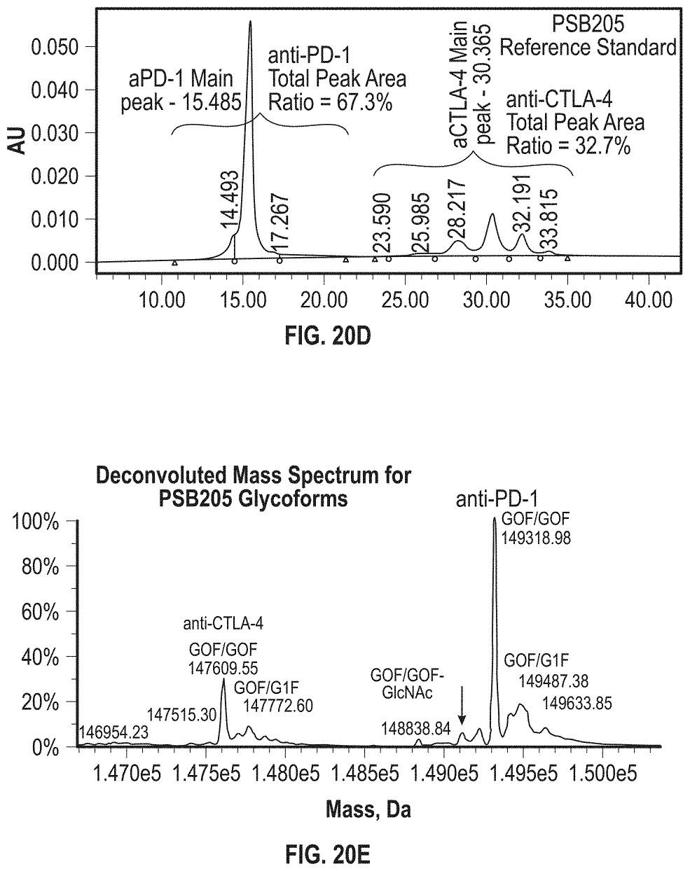

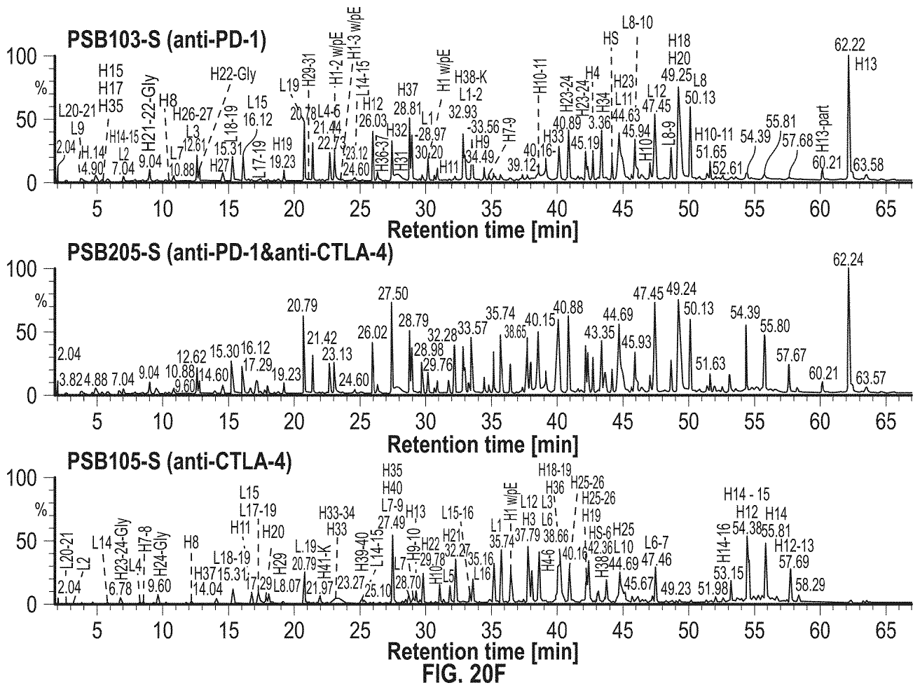

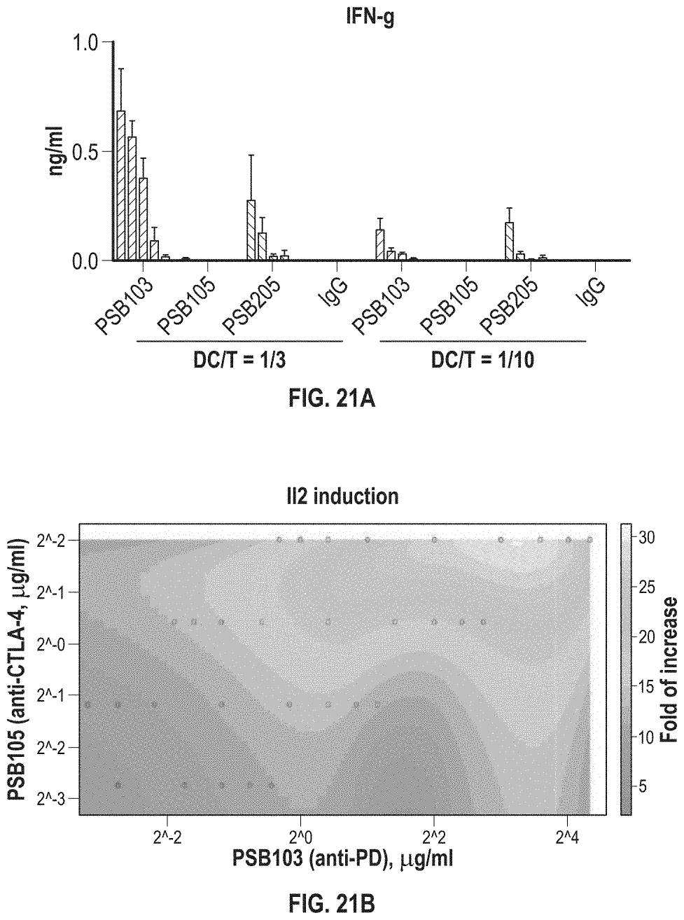

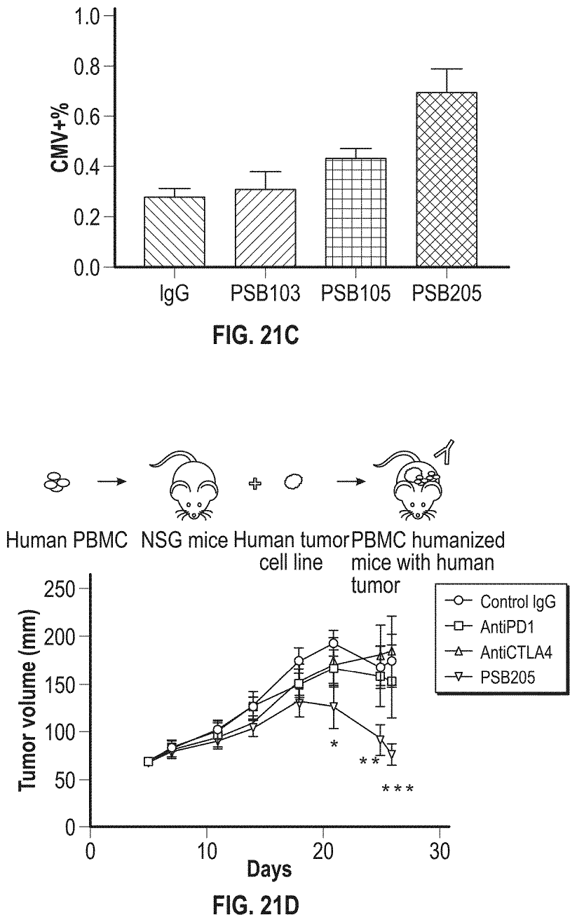

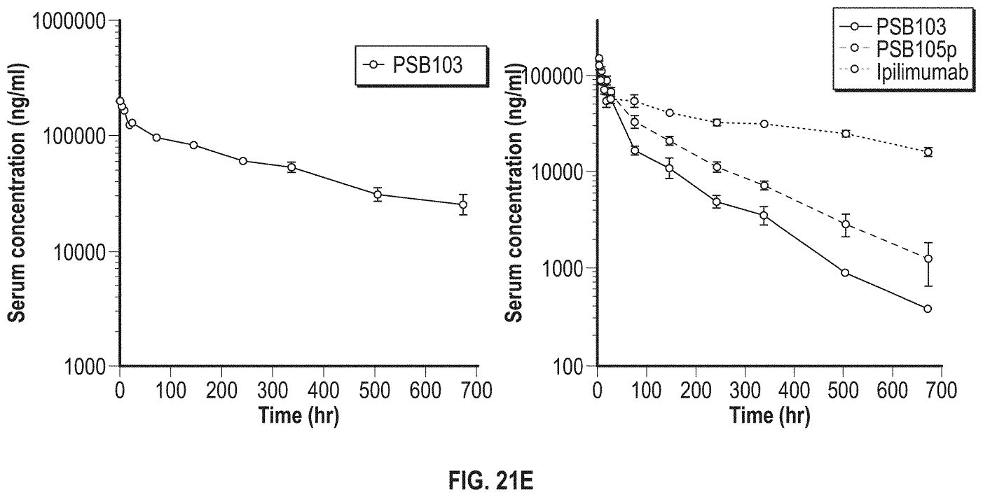

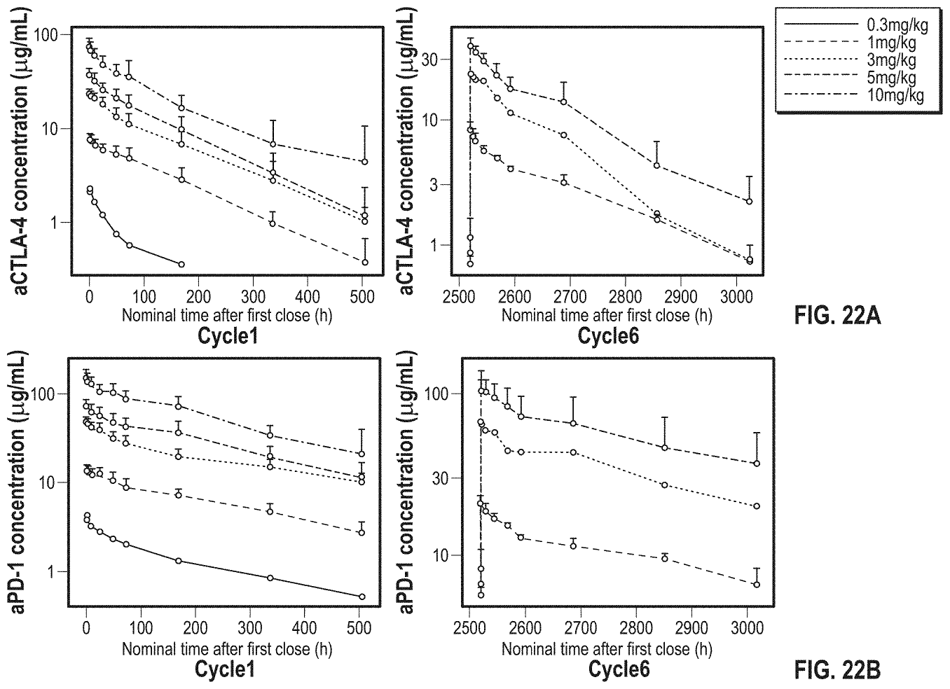

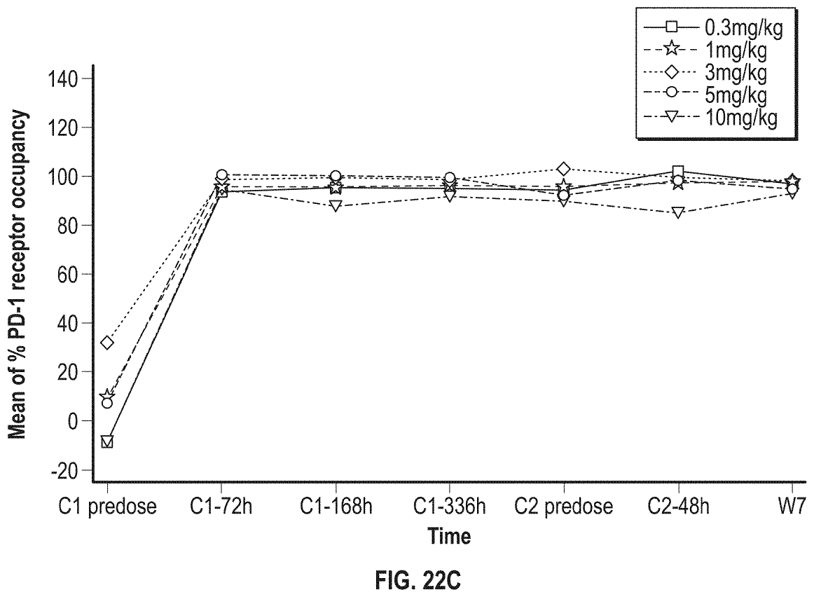

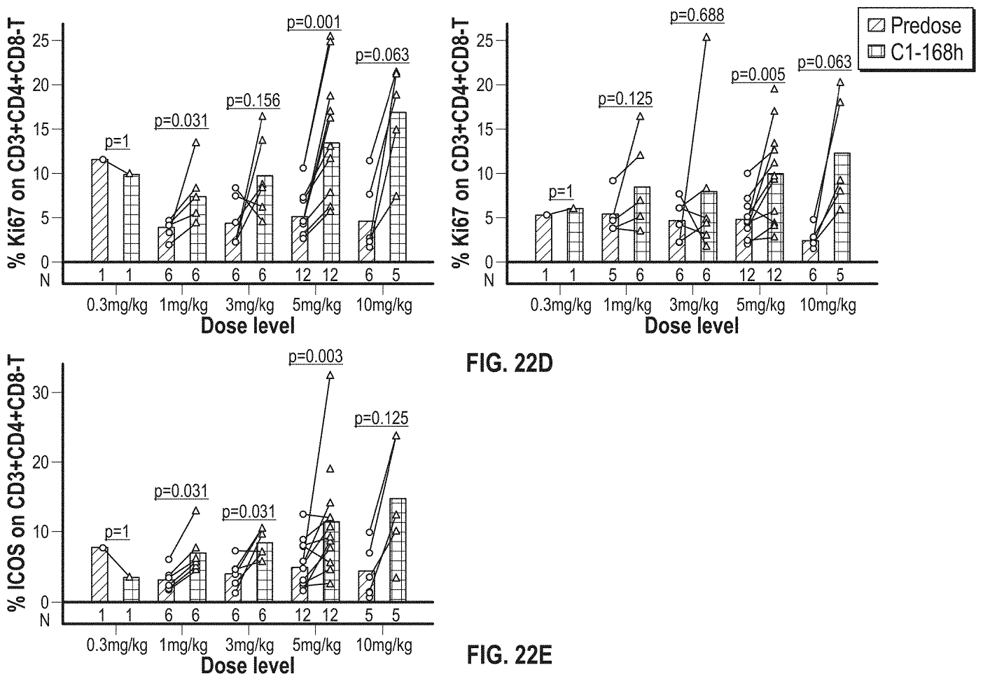

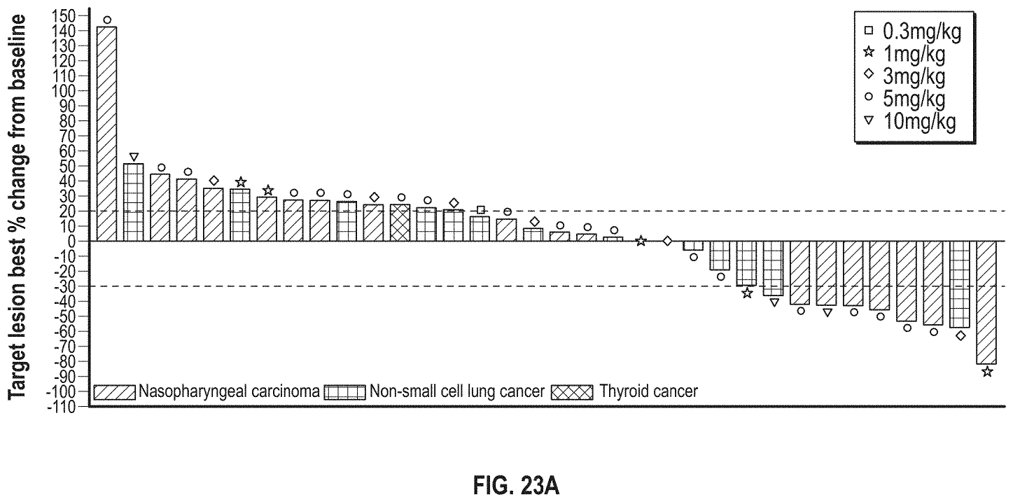

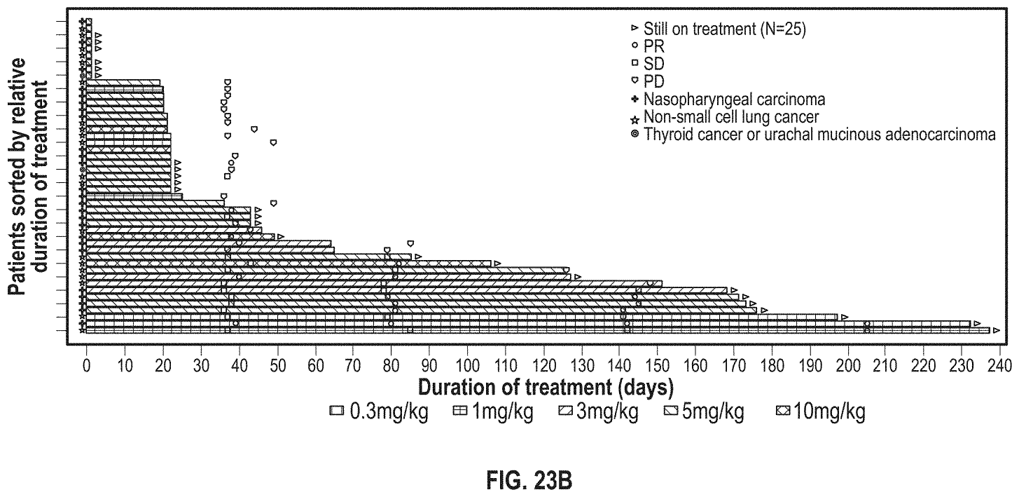

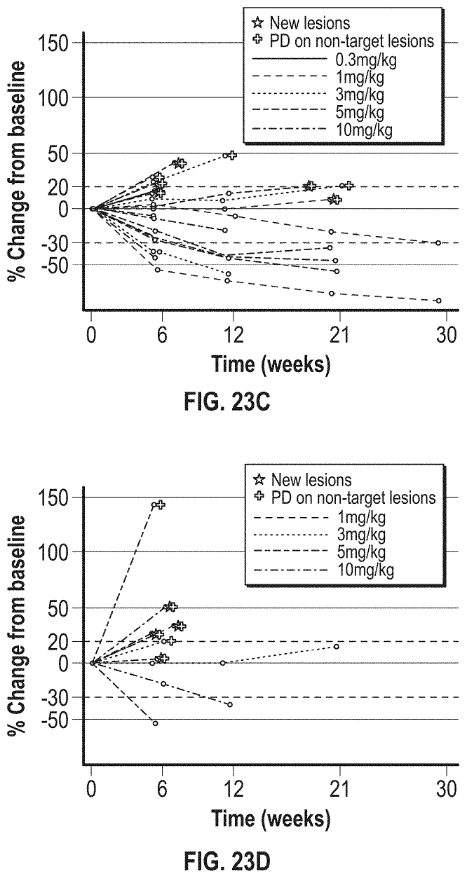

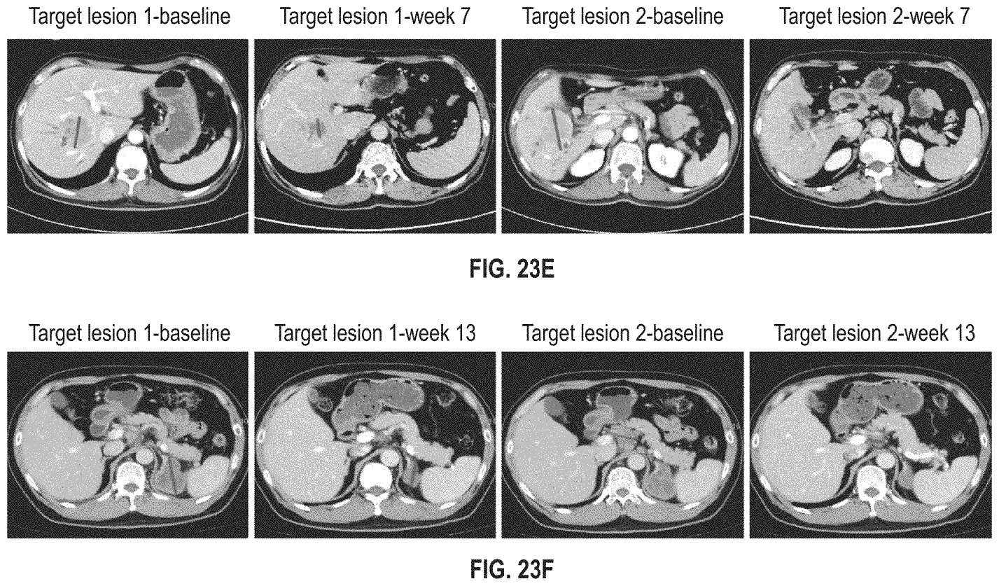

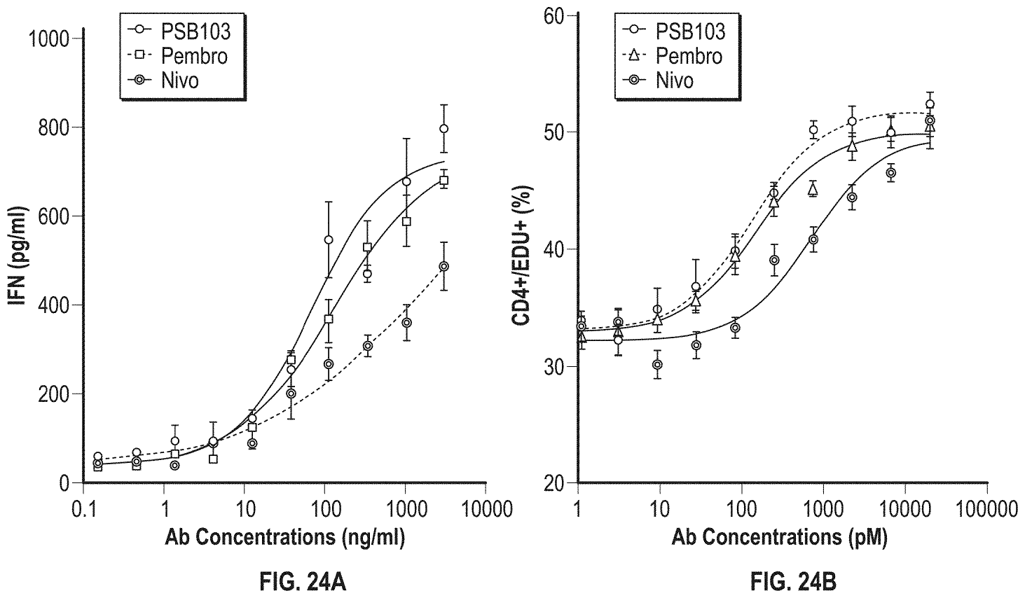

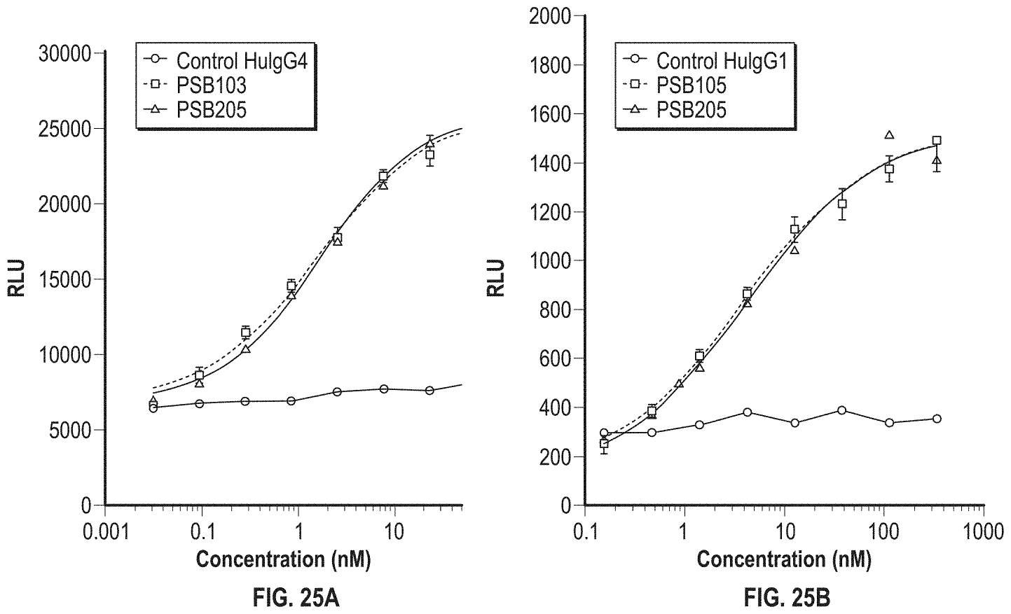

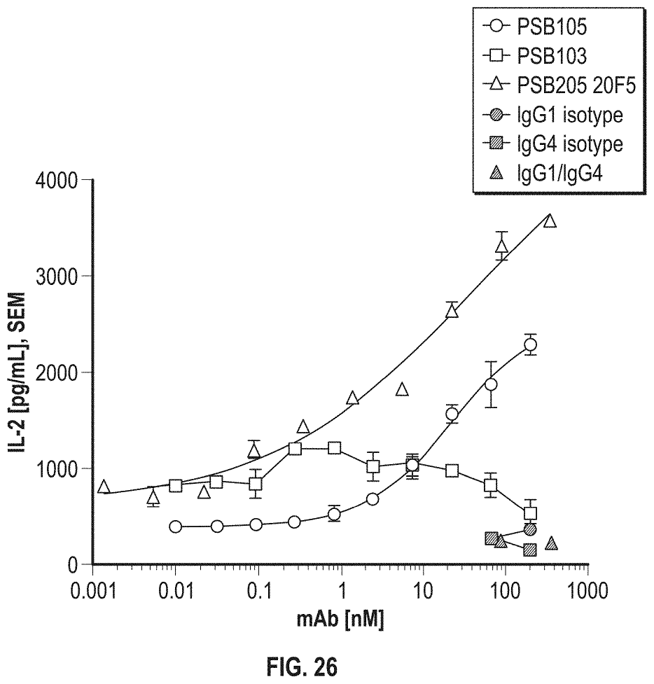

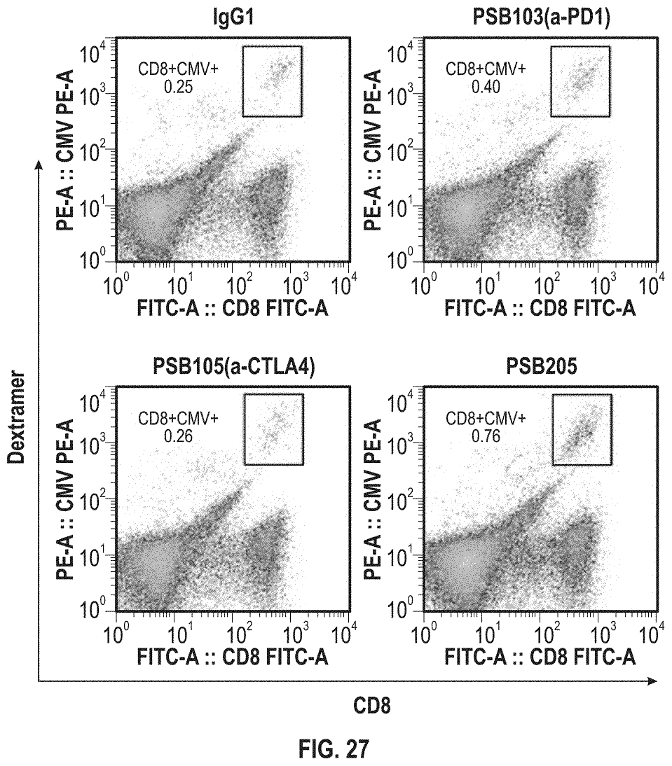

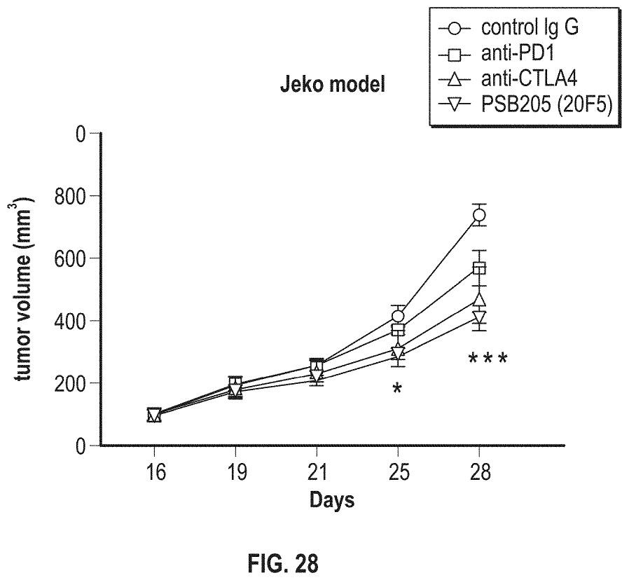

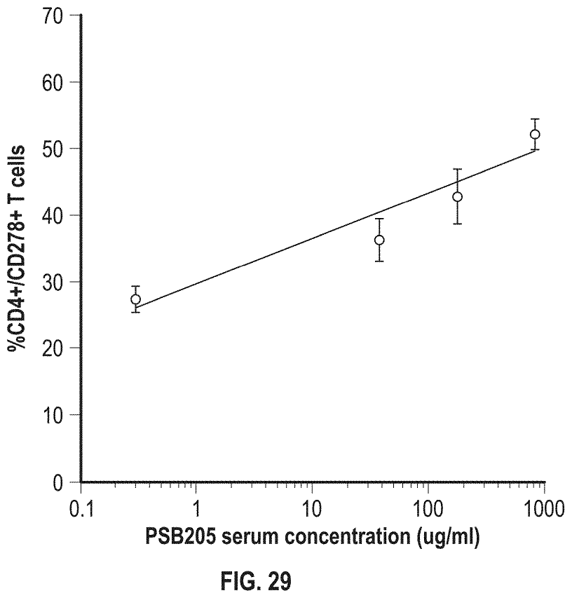

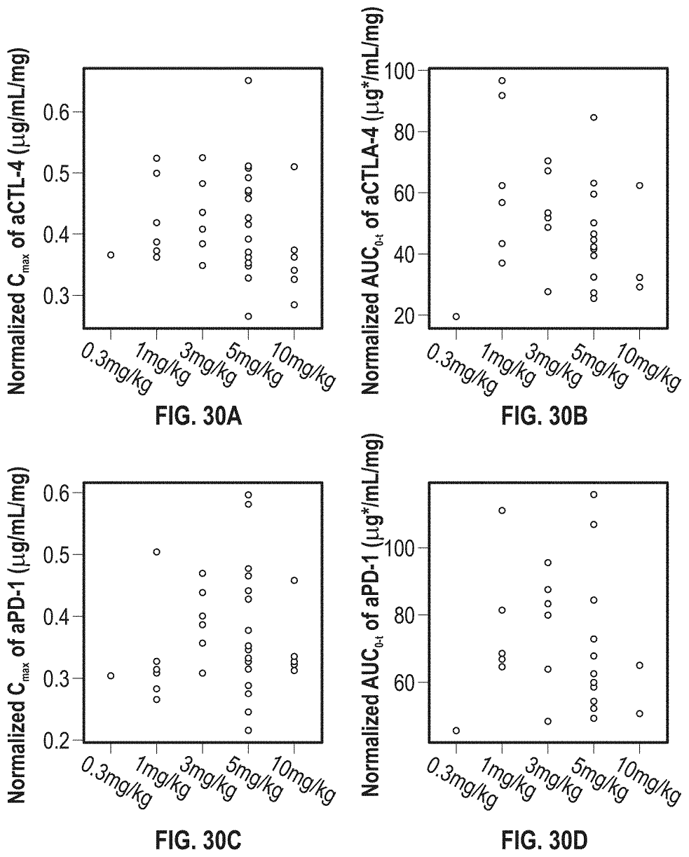

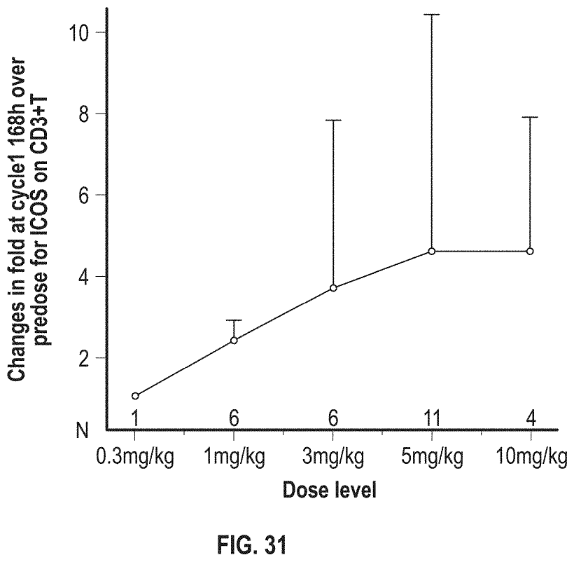

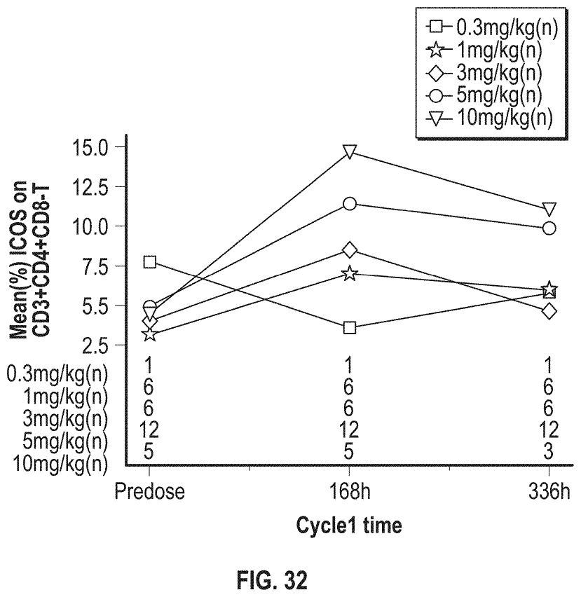

(FIG. 20E) Analysis of the intact glycoform mass profile of PSB205 by liquid chromatography—mass spectrometry (LC-MS). The two main peaks at 149,320 Da and 147,610 Da in the deconvoluted mass spectra closely match GOF/GOF glycoforms of anti-PD-1 and anti-CTLA-4, respectively. (FIG. 20F): Characterization of the two mAbs in PSB205 by LC-MS/MS peptide mapping. To distinguish peptides identified from either mAbs, purified individual anti-PD-1 and anti-CTLA-4 were also analyzed along with PSB205 sample. Most of the tryptic peptides of anti-PD-1 and anti-CTLA-4 were identified with peaks assigned in the two maps. The middle panel contains the peptide map of PSB205 (containing both anti-PD-1 and anti-CTLA-4), which represents the combined peptide map of the two individual mAb peptide maps. The protein sequences were confirmed with high confidence as the identified sequence coverage being 98.2% for anti-PD-1 and 95.8% for anti-CTLA-4.

FIGS. 21A-E: Preclinical assessments of PSB205. (FIG. 21A): Monocyte derived immature dendritic cells from a healthy donor were mixed with purified T cells from a different donor at 1:10 and 1:3 ratios in the presence of in the presence of 10 fold serial dilution of various antibodies (10 μg/ml to 0.001 μg/ml). At day 6, the levels of IFN gamma in the supernatant were evaluated by ELISA. (FIG. 21B): PBMC from a healthy donor was stimulated with SEB (100 ng/ml) for 96 hours in the presence of various concentrations of PSB103(0.5 μg/ml to 20 μg/ml) and PSB105 (0.05 ug/ml to 4 μg/ml) mixed at different ratios (5:1 to 0.2:1). The levels of IL-2 in the supernatant were determined by ELISA. The contour plot shows the fold of IL-2 increase at different concentrations of various ratios. Each data point is represented by the red dot on the graph. The bar on the right depicts the colorized representation of the fold of increase: white (highest) and green (lowest). The result is representative of three experiment from different donors. (FIG. 21C): PBMC from HLA-CMV pp65 positive donor was stimulated CMV (3 μg/mL) lysate for 7 days in the presence of various antibodies in duplicate: IgG1 (5 μg/mL), PSB103(5 μg/mL), PSB105(2.5 μg/mL), PSB205(5 μg/mL). The numbers of CMVpp65 positive CD8 T cells in the culture were enumerated by flow cytometry. (FIG. 21D): HCC827 were implanted on NCG mice. When the tumor sizes reached 60-80 mm\hat{ }3, human PBMC from a healthy donor was used to reconstitute NCG mice as described in the Method and Material. Control human IgG1(7.5 mg/kg n=5), PSB103(5 mg/kg n=5), PSB105(2.5 mg/kg n=5), and PSB103:mixed with PSB105 at 2:1 ratio (7.5 mg/kg n=5) were i.p. injected twice a week for 3 weeks. (FIG. 21E): Serum concentration vs. time curves for PSB103 (left), PSB105 and Ipilimumab (right) following single i.v. administration to cynomolgus monkeys at 5 mg/kg and 3 mg/kg respectively.

FIGS. 22A-E: Mean (+SD) plasma concentrations of aCTLA-4 (FIG. 22A) and aPD-1(FIG. 22B) as a function of time following dosing in Cycle 1 and at steady state (Cycle 6) shown on log 10 scale in μg/mL across dose levels from 0.3 mg/kg to 10 mg/kg Q3W. When more than half (>50%) of the values at a single time point are BQL, mean values are reported as 0. For those BQL values, they are omitted on the semi-log scale plot. When there are only 2 samples at a single time point, the error bars are not presented. (FIG. 22C): PD-1 Receptor occupancy in circulating CD3 T cells after PSB205 treatment. Average percentage of PD1 receptor occupancy was plotted at various timepoints before and after treatment of PSB205. (FIG. 22D) Proliferation of CD4 and CD8 T cells after PSB205 treatment. The percentage of Ki67+CD4 T cells (left panel) and CD8 T cells (right panel) before treatment or 168 hours post treatment were compared in each patient and linked by a line. The mean value; percentage of ICOS+CD4 T cells before or 168 hours after treatment were compared and linked by a line. The p value shown in (FIG. 22D) and (FIG. 22E) was calculated using Wilcoxon Signed-Rank Test.

FIGS. 23A-F: Tumor response. The best objective responses of target lesions from the baseline (FIG. 23A). One patient only got one post-baseline tumor assessment result and one of the target lesions could not be measured. Percentage change from baseline in tumor shrinkage in patients naïve to prior immunotherapy (FIG. 23B) and in patients with prior anti-PD-1/PD-L1 therapy (FIG. 23C). The dotted line at −30% indicates the threshold for a PR. Individual patient's duration of treatment (FIG. 23D). FIG. 23E shows a representative partial tumor response in a nasopharyngeal carcinoma patient in 5 mg/kg that was refractory to prior PD-L1/TGFβ bispecific inhibitor therapy. The sum of diameters for all target lesions was 101 mm at baseline and 47 mm at week 7 (−53.5%). FIG. 23F shows a representative partial tumor response in a non-small cell lung cancer patient in 10 mg/kg that was refractory to prior nivolumab and 4-1BB inhibitor therapy. The sum of diameters for all target lesions was 77 mm at baseline and 49 mm at week 13 (−36.4%).

FIGS. 24A-B: Both panels FIG. 24A and FIG. 24B show enhanced T Cell Activation in Allo-MLR of an anti-PD-1 IgG4, designated as PSB103. Ab=antibody; IFNγ=Interferon gamma; MLR=mixed lymphocyte reaction; Nivo=nivolumab; Pembro=pembrolizumab.

FIGS. 25A-B: shows that PSB205 and PSB103 inhibited PD-1 Binding and Functional Activity (FIG. 25A) and PSB205 and PSB105 inhibited CTLA4 mediated inhibitory activities (FIG. 25B) in dual cell reporter cell assays.

FIG. 26: shows that PSB205 Synergistically Enhanced T Cell Activation Induced by SEB Super Antigen.

FIG. 27: shows that PBMC from HLA-CMV pp65 positive donor was stimulated by CMV (3 ug/ml) lysate for 7 days in the presence of various antibodies in duplicate: IgG1 (5 ug/ml), PSB103(5 ug/ml), PSB105(2.5 ug/ml), PSB205(5 ug/ml). The numbers of CMVpp65 positive CD8 T cells in the culture were enumerated by flow cytometry.

FIG. 28: Jeko-1 were implanted on NCG mice. When the tumor sizes reached 80-100 mm\hat{ }3, human PBMC from a healthy donor was used to reconstitute NCG mice as described in the Method and Material. Control human IgG1(7.5 mg/kg n=5), PSB103(5 mg/kg n=5), PSB105(2.5 mg/kg n=5), and PSB103 mixed with PSB105 at 2:1 ratio (7.5 mg/kg n=5) were i.p. injected twice a week for 3 weeks.

FIG. 29: shows that PSB205 treatment increases levels of circulating CD4+/CD278+ T cells in cynomolgus monkeys. The estimated serum concentrations at day 16 (24 hrs after the 2nd dose) were plotted against the % of circulating CD4+/CD278+ T cells in the blood of PSB205-treated monkeys at day 16.

FIGS. 30A-D: shows individual Cmax normalized by actual dose for a CTLA-4 (FIG. 30A) and aPD-1 (FIG. 30C); and individual AUC0-t normalized by actual dose for aCTLA-4 (FIG. 30B) and aPD-1 (FIG. 30D) are shown as a function of the dose level.

FIG. 31: The expansion of ICOS+CD4+CD8− T cells after PSB205 treatment. The average percentage of ICOS+CD4 T cell in each dose group at different time points (Predose, 168 hours and 336 hours post treatment) were compared and linked with a solid line.

FIG. 32: The expansion of ICOS+CD4+CD8− T cells after PSB205 treatment. The average percentage of ICOS+CD4 T cell in each dose group at different time points (Predose, 168 hours and 336 hours post treatment) were compared and linked with a solid line.

### BRIEF DESCRIPTION OF THE SEQUENCE LISTING

| SEQ ID NO | Description of Sequence |
|---|---|
| SEQ ID NO: 1 | Amino acid sequence encoded by SEQ ID NO: 2 |
| SEQ ID NO: 2 | Nucleic acid sequence encoding the mature heavy chain (HC) of the anti-hPD1 |
| | antibody PSB103 |
| SEQ ID NO: 3 | Amino acid sequence encoded by SEQ ID NO: 4 |
| SEQ ID NO: 4 | Nucleic acid sequence encoding the mature V_{H} of the anti-hPD1 antibody |
| | PSB103 |
| SEQ ID NO: 5 | Amino acid sequence encoded by SEQ ID NO: 6 |
| SEQ ID NO: 6 | Nucleic acid sequence encoding the mature light chain (LC) of the anti-hPD1 |
| | antibody PSB103 |
| SEQ ID NO: 7 | Amino acid sequence encoded by SEQ ID NO: 8 |
| SEQ ID NO: 8 | Nucleic acid sequence encoding the mature V_{L} of the anti-hPD1 antibody |
| | PSB103 |
| SEQ ID NO: 9 | Amino acid sequence of the mature V_{H} of the anti-hPD1 antibody PSB103 with |
| | post-translational modifications |
| SEQ ID NO: 10 | Amino acid sequence of the mature HC of the anti-hPD1 antibody PSB103 |
| | with post-translational modifications |
| SEQ ID NO: 11 | Amino acid sequence of the mature V_{L} of the anti-hPD1 antibody PSB103 with |
| | post-translational modifications |
| SEQ ID NO: 12 | Amino acid sequence of the mature LC of the anti-hPD1 antibody PSB103 with |
| | post-translational modifications |
| SEQ ID NO: 13 | Amino acid sequence encoded by SEQ ID NO: 14 |
| SEQ ID NO: 14 | Nucleic acid sequence encoding the mature HC of the anti-hCTLA4 antibody |
| | PSB105 |
| SEQ ID NO: 15 | Amino acid sequence encoded by SEQ ID NO: 16 |
| SEQ ID NO: 16 | Nucleic acid sequence encoding the mature V_{H} of the anti-hCTLA4 antibody |
| | PSB105 |
| SEQ ID NO: 17 | Amino acid sequence encoded by SEQ ID NO: 18 |
| SEQ ID NO: 18 | Nucleic acid sequence encoding the mature LC of the anti-hCTLA4 antibody |
| | PSB105 |
| SEQ ID NO: 19 | Amino acid sequence encoded by SEQ ID NO: 20 |
| SEQ ID NO: 20 | Nucleic acid sequence encoding the mature V_{L} of the anti-hCTLA4 antibody |
| | PSB105 |
| SEQ ID NO: 21 | Amino acid sequence of the mature V_{H} of the anti-hCTLA4 antibody PSB105 |
| | with post-translational modifications |
| SEQ ID NO: 22 | Amino acid sequence of the mature HC of the anti-hCTLA4 antibody PSB105 |
| | with post-translational modifications |
| SEQ ID NO: 23 | Amino acid sequence of the mature V_{L} of the anti-hCTLA4 antibody PSB105 |
| | with post-translational modifications |
| SEQ ID NO: 24 | Amino acid sequence of the mature LC of the anti-hCTLA4 antibody PSB105 |
| | with post-translational modifications |

### DETAILED DESCRIPTION

Described herein are mixtures or combinations of antibodies that can be produced in a single cell line, where the antibodies in a mixture as described herein are an anti-hPD1 antibody and an anti-hCTLA4 antibody, each having specified sequences and properties. The antibodies are present in a specified ratio in the mixture. As evidenced by data provided herein, the mixture has advantageous properties as compared with either antibody alone, with a mixture of antibodies produced in two separate cell lines, and/or with other mixtures of anti-hPD1 and anti-hCTLA4 antibodies.

### Definitions

An “adverse event” (AE), as meant herein, is any unfavorable and unintended sign (including an abnormal laboratory finding), symptom, or disease temporally associated with the use of a medical treatment or procedure that may or may not be considered related to the medical treatment or procedure. AEs are classified as grades 1-5 AEs as follows: grade 1, a mild AE that is asymptomatic or includes mild symptoms observed clinically or in diagnostic tests, which does not indicate any intervention; grade 2, a moderate AE that includes minimal symptoms that may limit age-appropriate instrumental activities of daily living (ADL), which indicates local or noninvasive intervention; grade 3, a severe or medically significant AE that is not immediately life-threatening and that may be disabling or may limit ADL involved in self-care, which indicates a need for hospitalization or prolongation of hospitalization; grade 4, a life-threatening AE indicating urgent intervention; grade 5, an AE related to death. Immune-related adverse events (irAEs) are included within the ambit of what is meant by an AE herein. An irAE is temporally associated with drug treatment and can consist of the inflammation of any organ system in the body, most commonly the gastrointestinal tract, endocrine glands, skin, and liver. See, e.g., Postow et al. (2018), Immune-related adverse events associated with immune checkpoint blockade, New Engl. J. Med. 378: 158-168, available at DOI: 10.1056/NEJMra1703481, which is incorporated herein by reference. Inflammation of the central nervous system or cardiovascular, pulmonary, musculoskeletal and hematologic systems can also be part of an irAE. Id.

An “alteration,” as meant herein is a change in an amino acid sequence. Alterations can be insertions, deletions, or substitutions. An “alteration” is the insertion, deletion, or substitution of a single amino acid. If, for example, a deletion removes three amino acids from an amino acid sequence, then three alterations (in this case, deletions) have occurred. Alterations that are substitutions can be referred to by stating the amino acid present in the original sequence followed by the position of the amino acid in the original sequence followed by the amino acid replacing the original amino acid. For example, G133M means that the glycine at position 133 in the original sequence is replaced by a methionine. Further, 133M means that the amino acid at position 133 is methionine, but does not specify the identity of the original amino acid, which could be any amino acid including methionine. Finally, G133 means that glycine is the amino acid at position 133 in the original sequence.

An “antibody,” as meant herein, is a protein that contains at least one heavy chain (HC) variable domain (V_{H}) or light chain (LC) variable domain (V_{L}). An antibody often contains both a V_{H} and a V_{L}. V_{HS} and V_{LS} are described in full detail in, e.g., Kabat et al., SEQUENCES OF PROTEINS OF IMMUNOLOGICAL INTEREST, FIFTH EDITION, U.S. Department of Health and Human Services, Public Health Service, National Institutes of Health, NIH Publication No. 91-3242, 1991, pp. xvi-xix and pp. 103-533, which are incorporated by reference herein. “Antibody” includes molecules having different formats such as single chain Fv antibodies (scFv, which contain a V_{H} and a V_{L} joined by a linker), Fab, F(ab′)₂, Fab′, scFv:Fc antibodies (as described in Carayannopoulos and Capra, Ch. 9 in FUNDAMENTAL IMMUNOLOGY, 3.sup.rd ed., Paul, ed., Raven Press, New York, 1993, pp. 284-286, which is incorporated herein by reference), and IgG antibodies as defined below, among many other possible formats.

A “bispecific T cell engager (BiTE),” as meant herein, is described in, for example, Huehls et al. (2015), Bispecific T cell engagers for caner immunotherapy, Immunol. Cell Biol. 93(3): 290-296, which is incorporated herein by reference.

A “chemotherapeutic agent” targets dividing cells and interferes with processes that are tied to cell division, for example, DNA replication, RNA synthesis, protein synthesis, the assembly, disassembly, or function of the mitotic spindle, and/or the synthesis or stability of molecules that play a role in these processes, such as nucleotides or amino acids. Thus, a chemotherapeutic agent can kill both cancer cells and other dividing cells. Chemotherapeutic agents are well-known in the art. They include, for example, the following agents: alkylating agents (e.g., busulfan, temozolomide, cyclophosphamide, lomustine (CCNU), streptozotocin, methyllomustine, cis-diamminedichloroplatinum, thiotepa, and aziridinyl benzoquinone); inorganic ions (e.g., cisplatin and carboplatin); nitrogen mustards (e.g., melphalan hydrochloride, chlorambucil, ifosfamide, and mechlorethamine HCl); nitrosoureas (e.g., carmustine (BCNU)); anti-neoplastic antibiotics (e.g., adriamycin (doxorubicin), daunomycin, mithramycin, daunorubicin, idarubicin, mitomycin C, and bleomycin); plant derivatives (e.g., vincristine, vindesine, vinblastine, vinorelbine, paclitaxel, docetaxel, VP-16, and VM-26); antimetabolites (e.g., methotrexate with or without leucovorin, 5-fluorouracil with or without leucovorin, 5-fluorodeoxyuridine, 6-mercaptopurine, 6-thioguanine, gemcitabine, cytarabine, 5-azacytidine, hydroxyurea, deoxycoformycin, and fludarabine); podophyllotoxins (e.g., etoposide, irinotecan, and topotecan); as well as actinomycin D, dacarbazine (DTIC), mAMSA, procarbazine, hexamethylmelamine, pentamethylmelamine, L-asparaginase, and mitoxantrone. See, e.g., Cancer: Principles and Practice of Oncology, 4.sup.th Edition, DeVita et al., eds., J.B. Lippincott Co., Philadelphia, Pa. (1993), the relevant portions of which are incorporated herein by reference.

Other chemotherapeutic agents include those that act by the same general mechanism as those listed above. For example, agents that act by alkylating DNA, as do, for example, alkylating agents and nitrogen mustards, are considered chemotherapeutic agents. Agents that interfere with nucleotide synthesis, like, for example, methotrexate, cytarabine, 6-mercaptopurine, 5-fluorouracil, and gemcitabine, are considered to be chemotherapeutic agents. Mitotic spindle poisons are considered chemotherapeutic agents, as are, for, example, paclitaxel and vinblastine. Topoisomerase inhibitors (e.g., podophyllotoxins), which interfere with DNA replication, are considered to be chemotherapeutic agents. Antibiotics that interfere with DNA synthesis by various mechanisms, examples of which are doxorubicin, bleomycin, and mitomycin, are considered to be chemotherapeutic agents. Agents that carbamoylate amino acids (e.g., lomustine, carmustine) or deplete asparagine pools (e.g., asparaginase) are also considered chemotherapeutic agents. Merck Manual of Diagnosis and Therapy, 17.sup.th Edition, Section 11, Hematology and Oncology, 144. Principles of Cancer Therapy, Table 144-2 (1999). Specifically included among chemotherapeutic agents are those that directly affect the same cellular processes that are affected by the chemotherapeutic agents listed above.

A “cognate” HC in the context of a mixture of antibodies, as meant herein, is the HC that a particular LC is known to pair with to form a binding site for a particular antigen. For example, if a known full-length IgG Antibody X binds to Antigen X, then the Antibody X HC is the cognate HC of the Antibody X LC, and vice versa. Further, if a mixture of antibodies comprises both Antibody X and Antibody Y, which binds to Antigen Y, the antibody Y HC is “non-cognate” with respect to the Antibody X LC and vice versa, and the Antibody Y LC is “non-cognate” with respect to the Antibody X HC and vice versa.

A “complementarity determining region” (CDR) is a hypervariable region within a V_{H} or V_{L}. Each V_{H} and V_{L} contains three CDRs called CDR1, CDR2, and CDR3. The CDRs form loops on the surface of the antibody and are primarily responsible for determining the binding specificity of an antibody. The CDRs are interspersed between four more conserved framework regions (called FR1, FR2, FR3, and FR4) as follows: FR1-CDR1-FR2-CDR2-FR3-CDR3-FR4. Kabat et al. position the V_{H} CDRs as follows: CDR1 is at positions 31-35 (with possible insertions numbered 35A and 35B); CDR2 is at positions 50-65 (with possible insertions numbered 52A-52C); and CDR3 is at positions 95-102 (with possible insertions numbered 100A-100K). Kabat et al., supra, at xvii. These positions of heavy chain CDRs are used herein except that the V_{H} CDR1 is considered to include positions 26-35 herein. Kabat et al. position the V_{L} CDRs as follows: CDR1 is at positions 24-34 (with possible insertions numbered 27A-27F); CDR2 is at positions 50-56; and CDR3 is at positions 89-97 (with possible insertions numbered 95A-95F). Kabat et al., supra, at xvii, which is incorporated herein by reference. These positions of CDRs with a V_{L} are used herein. The numbering scheme used immediately above is that utilized by Kabat et al., and it is exemplified in Kabat et al., supra, at pp. 103-539.

A treatment or drug is considered to be administered “concurrently” with another treatment or drug if the two treatments/drugs are administered within the same small time frame, for example on the same day, or within the same more extended time frame. Such a more extended time frame can include a situation where, for example, one treatment/drug is administered once per week and the other is administered every 4 days. Although the two treatments/drugs may never or rarely be administered on the same day, the two treatments/drugs are administered on an ongoing basis during a common period of weeks, months, or longer. Similarly, if one drug is administered once per year and the other is administered weekly, they are considered to be administered “concurrently” if the drug administered weekly is administered during the year before and/or after the administration of the drug that is administered once per year. Hence, as meant herein, “concurrent” administration of the two treatments/drugs includes ongoing treatment with two different treatments/drugs that goes on in a common time period.

A “complete response” (CR), when used in connection with a cancer patient undergoing treatment, is assessed as described in the RECIST guidelines (version 1.1). Eisenhauer et al. (2009), New response evaluation criteria in solid tumours: revised RECIST guideline (version 1.1.), Eur. J. Cancer 45: 228-247, which is incorporated herein in its entirety. These guidelines describe evaluating overall tumor burden by measuring diameters of all measurable tumors, up to a maximum of five tumors, at baseline. These diameters are added to determine a sum. These measured tumors are called the “target lesions.” In a CR, all target lesions have become undetectable during the course of the study.

A “partial response” (PR), when used in connection with a cancer patient undergoing treatment, is also assessed as described in the RECIST guidelines (version 1.1). In a patient rated as having a PR, the sum of the diameters of the target lesion(s) has decreased by at least 30% in the course of the treatment as compared to the sum of these diameters at baseline.

“Progressive disease” (PD), when used in connection with a cancer patient undergoing treatment, is also assessed as described in the RECIST guidelines (version 1.1). In a patient rated as having PD, the sum of the diameters of the target lesion(s) has increased by at least 20% as compared to the smallest sum detected during the course of the study. This smallest sum may be the sum detected at baseline or a sum detected later in the study. In addition, the sum that is increased by at least 20% must also demonstrate an absolute increase of at least 5 mm. The appearance of one or more new tumors is also considered to be PD.

“Stable disease” (SD), when used in connection with a cancer patient undergoing treatment, is also assessed as described in the RECIST guidelines (version 1.1). SD means that the sum of diameters of the target lesions has neither shrunk enough to qualify as a PR nor increased enough to qualify as PD when compared to the smallest sum of diameters detected during the course of the study. This smallest sum may be the sum detected at baseline or a sum detected later in the study.

An “objective response rate” (ORR) is the sum of the percent of patients achieving a PR and the percent of patients achieving a CR.

A “disease control rate” (DCR) is the sum of the percent of patients achieving a PR, the percent of patients achieving a CR, and the percent of patients achieving SD.

As meant herein, a first nucleic acid sequence “encodes” an amino acid sequence when, according to the genetic code, the first nucleic acid sequence could, when transcribed and translated, provide a blueprint for producing a protein comprising the amino acid sequence. The first nucleic acid sequence also “encodes” an amino acid sequence comprised by a protein produced by host cells into which a polynucleotide comprising the first nucleic acid sequence has been introduced, but not produced by the same host cells that do not contain the polynucleotide comprising the first nucleic acid sequence. Such an amino acid sequence will be largely as predicted by the genetic code, but may (or may not) comprise post-translational modifications that change the amino acid sequence. Such a slightly-altered amino acid sequence is, in fact, encoded by the first nucleic acid sequence and is considered herein to be encoded by the first nucleic acid sequence, which actually served as a blueprint for its production, even though it may comprise minor variations from a predicted amino acid sequence.

An “Fc fragment,” “Fc region,” or “Fc portion,” as meant herein, consists essentially of a hinge domain (hinge), a second HC constant domain (C_{H}2), and a third HC constant domain (C_{H}3) from an HC, although it may further comprise regions, for example a fourth HC constant domain (C_{H}4), downstream from the C_{H}3 in some isotypes such as IgA or IgM.

A “heavy chain (HC),” as meant herein, comprises at least a V_{H}, a first HC constant domain (C_{H}1), a hinge, a C_{H}2, and a C_{H}3. An HC including all of these domains could also be referred to as a “full-length HC” or an “IgG HC” (in a case where the HC is of the IgG isotype). Some isotypes such as IgA or IgM can contain additional sequences, such as the IgM C_{H}4 domain.

A “human,” nucleotide or amino acid sequence, protein, or antibody is one that occurs naturally in a human or one that is identical to such a sequence or protein except for a small number of alterations as explained below. Many human nucleotide and amino acid sequences are reported in, e.g., Kabat et al., supra, which illustrates the use of the word “human” in the art. A “human” amino acid sequence or antibody, as meant herein, can contain one or more insertions, deletions, or substitutions relative to a naturally-occurring sequence, with the proviso that a “human” amino acid sequence does not contain more than 10 insertions, deletions, and/or substitutions of a single amino acid per every 100 amino acids in a naturally-occurring sequence. Similarly, a human nucleotide sequence does not contain more than 30 insertions, deletions, and/or substitutions of a single nucleotide per every 300 nucleotides in a naturally-occurring sequence. In the particular case of a V_{H} or V_{L} sequence, the CDRs are expected to be extremely variable, and, for the purpose of determining whether a particular V_{H} or V_{L} amino acid sequence (or the nucleotide sequence encoding it) is a “human” sequence, the CDRs (or the nucleotides encoding them) are not considered part of the sequence.

A “humanized” antibody, as meant herein, is an antibody where the antibody is of non-human origin but has been engineered to be human as much as possible, thereby hopefully reducing immunogenicity in humans while retaining antibody stability and binding properties. Generally, this means that most or all of the constant domains and the framework regions of the variable domains are human, or nearly human sequences, while the CDRs originate from a different organism. However, merely grafting CDRs from, e.g., a mouse antibody, into a human framework may not produce an antibody with the desired properties, and further modification may be required. In recent years, a variety of approaches to streamline and improve the results of humanization have been developed. See, e.g., Choi et al. (2015), mAbs 7(6): 1045-1057 and references cited therein.

An “IgG antibody,” as meant herein, comprises (1) two HCs, each comprising a V_{H}, a C_{H}1, a hinge, a C_{H}2, and a C_{H}3 and (2) two LCs, each comprising a V_{L} and an LC constant domain (C_{L}). The heavy chains of an IgG antibody are of an IgG isotype, for example, IgG1, IgG2, IgG3, or IgG4. These domains are described in, e.g., Kabat et al., supra, pp. xv-xix and 647-699, which pages are incorporated herein by reference. The C_{L} can be a kappa (C_{L}κ) or lambda (C_{L}λ) domain.

An “immunomodulatory molecule,” as meant herein, is a molecule that interacts with a component, for example a protein, that can mediate the activity of the immune system, thereby regulating the activity of the immune system. The activity of the immune system can be assessed in a cytomegalovirus (CMV) recall response assay as described in Example 8 below, and an immunomodulatory molecule can either increase or decrease activity in this assay relative to a negative control molecule. As an example, the anti-hPD1 PSB103 antibody and the PSB205 antibody mixture described herein are immunomodulatory molecules by this definition.

A “light chain (LC),” as meant herein, comprises a V_{L} and a C_{L}, which can be a C_{L}κ or C_{L}λ. These domains, including exemplary amino acid sequences thereof, are described in, e.g., Kabat et al., supra, pages xiii-lix, 103-309, and 647-660, which are incorporated herein by reference.

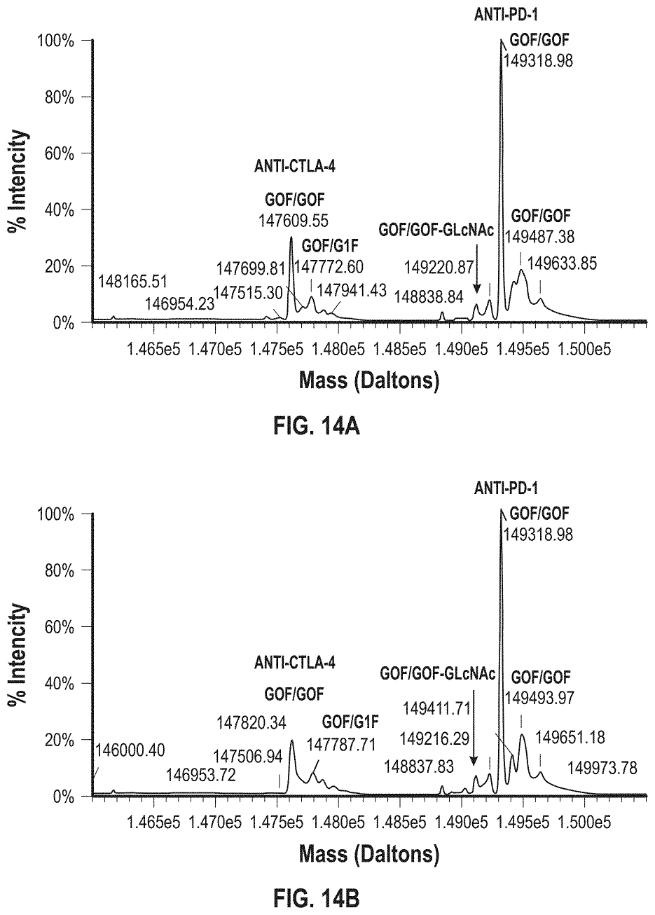

A “major species” of antibody in the context of a mixture of antibodies, as meant herein, is a particular antibody that makes up at least 10% of the total amount of antibodies within the mixture. To determine how many major species are in a mixture of antibodies, low pH cation exchange (CEX) chromatography as described in Example 5 and shown in FIG. 14 of U.S. Provisional Application 62/342,167 (which portions of U.S. Provisional Application 62/342,167 are incorporated herein by reference) can be performed. This method is described by Chen et al. (2010), Protein Science, 19:1191-1204, which is incorporated herein in its entirety. Briefly, it employs a Thermo PROPAC™ WCX-10 weak CEX column, 4×250 mm, preceded by a 50 mm guard column (PROPAC™ WCX-10G) using a Waters Alliance 2695 high performance liquid chromatography (HPLC) system. Chromatography can be run with a linear gradient from 100% Buffer A (20 mM sodium acetate pH 5.2) to 100% Buffer B (20 mM sodium acetate with 250 mM sodium chloride pH 5.2) over 30 minutes. The column can be washed with high salt (1M sodium chloride) and re-equilibrated to starting condition of Buffer A. Antibodies can be detected in the column outflow by absorbance at 214 nm. Relative amounts of the detected peaks can be determined using EMPOWER™ software (Waters Corp., Milford, Mass., USA). Low pH CEX can distinguish between different full-length antibody species and can be used to quantitate relative amounts of specific antibody species in a mixture.

A “minor species” of antibody within a mixture of antibodies, as meant herein, comprises less than 10% of the total amount of antibodies in the mixture. This can be determined by low pH CEX chromatography as described in the definition of “major species.”

The terms “nucleic acid” and “polynucleotide” are used interchangeably herein.

An “oncolytic virus,” as meant herein, is a virus that preferentially lyses cancer cells as compared to normal cells. Oncolytic viruses can be naturally occurring or can be constructed in a laboratory. Examples of oncolytic viruses include adenovirus, reovirus, measles virus, herpes simplex virus, Newcastle disease virus, and vaccinia virus.

“PSB103,” as meant herein, refers to an anti-hPD1 IgG4 antibody encoded by DNA(s) encoding the amino acid sequences of SEQ ID NOs: 1 and 5.

“PSB105,” as meant herein, refers to an anti-hCTLA4 IgG1 antibody encoded by DNA(s) encoding the amino acid sequences of SEQ ID NOs: 13 and 17.

“PSB205,” as meant herein, is antibody mixture of PSB105 and PSB103, as defined above, wherein the weight/weight (w/w/) ratio of PSB105:PSB103 in the mixture is, respectively, from 1:1 to 1:4 and wherein the mixture contains no major species of antibodies other than PSB105 and PSB103.

“Radiation,” as meant herein, is used in the treatment of cancer. Radiation treatments, as meant herein, can include external beam radiation using, for example, photon, proton, or electron beams, and/or internal radiation. There are many kinds of external radiation, including, e.g., 3-D conformational radiation therapy, intensity-modulated radiation therapy (IMRT), image-guided radiation therapy (IGRT), TOMOTHERAPY®, stereotactic radiosurgery, and stereotactic body radiation therapy. Internal radiation methods include, for example, brachytherapy or systemic administration using a radioactive substance, e.g., radioactive iodine.

A “single dose study” in cynomolgus monkeys, as meant herein, is a pharmacokinetic study where only one dose of a drug being tested is administered to the monkeys. Such a study is performed essentially as described in Example 3, with the understanding that minor variations from the methods described in Example 3, for example variations in the dose of the drug administered or the number of monkeys dosed with the drug, would still be within what is considered a “single dose study” as meant herein.

An “in vivo half life (t_{1/2}) in a single dose study in cynomolgus monkey,” as meant herein, is determined essentially as described in Example 3 below. An in vivo t_{1/2} resulting from a protocol having minor variations in from that described in Example 3, such as, for example, testing different doses and/or testing different numbers of monkeys, is also considered an “in vivo t_{1/2} in a single dose study in cynomolgus monkey” as meant herein.

A “targeted biologic,” as meant herein, is a protein that can influence an aspect of a cell's biological status via its interaction with another specific molecule (which can be a protein). For example, a “targeted biologic” may influence a cell's ability to live, to proliferate, to produce specific cytokines or proteins, etc. As an example, the anti-hPD1 antibodies described herein are “targeted biologics” since they interact with PD1, which causes a number of biological effects in T cells including an increase in proliferation and an increase in IFNγ production.

A “targeted inhibitor,” as meant herein, is small molecule that can influence an aspect of a cell's biological status via its interaction with a specific cellular molecule (which can be a protein). For example, a “tyrosine kinase inhibitor” is a small molecule that affects the activity of tyrosine kinase (which affects a variety of cell functions) via its interaction with tyrosine kinase.

As meant herein, a “treatment” for a particular disease or condition refers to a course of action, which can comprise administration of one or more antibodies or polynucleotides encoding one or more antibodies, that results in a lessening of one or more symptoms or a decrease or interruption in an expected progression of the disease or condition in a human patient or in an animal model system considered to be reflective of the disease or condition. Alternatively or in addition, a treatment can alter results of an in vitro cell-based assay considered to be reflective of the disease or condition. These differences can be ascertained by an objective measurement of symptoms in humans or animals or by measurement of various parameters in cell-based assays, for example, production of one or more cytokines, e.g., IFNγ, cell proliferation, cell death, proliferation of cytotoxic immune cells, e.g., T cells, etc. For example, for a cancer “treatment,” the treatment can result in a decrease in tumor volume, an absence of expected tumor metastasis in a human or in an animal model system, an increase in survival time, or an increase in progression-free or disease-free survival time in a human or animal suffering from cancer. A cancer treatment may result in an increase in indices indicating activation of the immune system in a cell-based assay, for example, increased number of antigen-specific T cells and/or increased production of cytokines, e.g., IFNγ and/or IL-2, by T cells.

### An Anti-hPD1 Antibody

An anti-hPD1 antibody as described herein can be a human or humanized IgG antibody. The HC of an anti-hPD1 antibody as described herein can be a human or humanized IgG HC, such as an IgG1, IgG2, IgG3, or IgG4 HC. In some embodiments, this HC is an IgG4 HC. In one aspect, this HC can be encoded by the nucleic acid sequence of SEQ ID NO: 2. Exemplary amino acid sequences that are encoded by SEQ ID NO: 2 include SEQ ID NO: 1 and/or SEQ ID NO: 10 or amino acid sequences comprising four, three, two, or one alteration(s) relative to SEQ ID NO: 1 or SEQ ID NO: 10. An amino acid sequence encoded by a polynucleotide can comprise post-translational alterations that alter its sequence relative to, for example, the amino acid sequence predicted by the genetic code. The exact nature of such post-translational modifications can depend on the nature of the host cell in which an antibody is produced. An example of such an amino acid sequence is SEQ ID NO: 10, which reflects actual post-translational modifications found in the HC of an anti-hPD1 antibody made in a CHO host cell. See Example 6 below. To be clear, an anti-hPD1 antibody comprising an HC having the amino acid sequence of SEQ ID NO: 10 can be produced in host cells, for example CHO cells, containing a nucleic acid encoding an HC comprising the amino acid sequence of SEQ ID NO: 1. In another aspect, the amino acid sequence of an HC of an anti-hPD1 antibody as described herein can comprise no more than ten, nine, eight, seven, six, five, four, three, two, one, or zero alteration(s) relative to SEQ ID NO: 1 or SEQ ID NO: 10, regardless of the nucleic acid sequence encoding the amino acid sequence of the HC.

The V_{H} of an anti-hPD1 in the mixture can be encoded by a nucleic acid sequence encoding the amino acid sequence of, for example, SEQ ID NO: 3 and/or SEQ ID NO: 9 or a nucleic acid sequence encoding an amino acid sequence comprising four, three, two, or one alteration(s) relative to SEQ ID NO: 3 and/or SEQ ID NO: 9. One such nucleic acid sequence is SEQ ID NO: 4. An amino acid sequence encoded by SEQ ID NO: 4 can comprise post-translational alterations that alter its sequence relative to, for example, SEQ ID NO: 3. The exact nature of such post-translational modifications can depend on the nature of the host cell in which an antibody is produced. An example of such an amino acid sequence is SEQ ID NO: 9, which reflects actual post-translational modifications found in the V_{H} of an anti-hPD1 antibody made in a CHO host cell. See Example 6 below. To be clear, an anti-hPD1 antibody comprising a V_{H} having the amino acid sequence of SEQ ID NO: 9 can be produced in host cells, for example CHO cells, containing a nucleic acid encoding a V_{H} comprising the amino acid sequence of SEQ ID NO: 3. In another aspect, the amino acid sequence of a V_{H} of an anti-hPD1 antibody as described herein can comprise four, three, two, one, or zero alteration(s) relative to SEQ ID NO: 3 or SEQ ID NO: 9, regardless of the nucleic acid sequence encoding the amino acid sequence of the V_{H}.

In another aspect, an anti-hPD1 antibody as described herein can comprise a V_{H} encoded by a nucleic acid sequence encoding SEQ ID NO: 3 and can comprise constant domains having amino acid sequences other than those included in SEQ ID NO: 1, for example, constant domains from an IgG1, IgG2, or IgG3 antibody, which may or may not be a human or humanized antibody.

The LC of an anti-hPD1 antibody as described herein can be a human or humanized IgG LC comprising a C_{L}κ or C_{L}λ. In some embodiments, the C_{L} comprises a C_{L}κ. The LC of an anti-hPD1 antibody as described herein can be encoded by a nucleic acid sequence encoding the amino acid sequence of SEQ ID NO: 5 and/or SEQ ID NO: 12 or a nucleic acid sequence encoding an amino acid sequence comprising four, three, two, or one alteration(s) relative to SEQ ID NO: 5. One such nucleic acid sequence is SEQ ID NO: 6. An amino acid sequence encoded by SEQ ID NO: 6 can comprise post-translational alterations that alter its sequence relative to, for example, SEQ ID NO: 5. The exact nature of such post-translational modifications can depend on the nature of the host cell in which an antibody is produced. An example of such an amino acid sequence is SEQ ID NO: 12, which reflects actual post-translational modifications found in the LC of an anti-hPD1 made in a CHO host cell. See Example 6 below. To be clear, an anti-hPD1 antibody comprising an LC having the amino acid sequence of SEQ ID NO: 12 can be produced in host cells, for example CHO cells, containing a nucleic acid encoding an LC comprising the amino acid sequence of SEQ ID NO: 5. In another aspect, the amino acid sequence of an LC of an anti-hPD1 antibody as described herein can comprise four, three, two, one, or zero alteration(s) relative to SEQ ID NO: 5 or SEQ ID NO: 12, regardless of the nucleic acid sequence encoding the amino acid sequence of the LC.

The V_{L} of an anti-hPD1 antibody as described herein can be encoded by a nucleic acid sequence encoding the amino acid sequence of SEQ ID NO: 7 and/or SEQ ID NO: 11 or a nucleic acid sequence encoding an amino acid sequence comprising four, three, two, or one alteration(s) relative to SEQ ID NO: 7. One such nucleic acid sequence is SEQ ID NO: 8. An amino acid sequence encoded by SEQ ID NO: 8 can comprise post-translational alterations that alter its sequence relative to, for example, SEQ ID NO: 7. The exact nature of such post-translational modifications can depend on the nature of the host cell in which an antibody is produced. An example of such an amino acid sequence is SEQ ID NO: 11, which reflects actual post-translational modifications found in the V_{L} of an anti-hPD1 antibody made in a CHO host cell. See Example 6 below. To be clear, an anti-hPD1 antibody comprising a V_{L} having the amino acid sequence of SEQ ID NO: 11 can be produced in host cells, for example CHO cells, containing a nucleic acid encoding a V_{L} comprising the amino acid sequence of SEQ ID NO: 7. In another aspect, the amino acid sequence of a V_{L} of an anti-hPD1 antibody as described herein can comprise four, three, two, one, or zero alteration(s) relative to SEQ ID NO: 7 or SEQ ID NO: 11, regardless of the nucleic acid sequence encoding the amino acid sequence of the V_{H}.

In another aspect, an anti-hPD1 antibody as described herein can comprise a V_{L} encoded by a nucleic acid sequence encoding SEQ ID NO: 7 and can comprise a C_{L} having an amino acid sequence other than that included in SEQ ID NO: 5. For example, such a C_{L} can be a lambda or a kappa C_{L}, which may or may not be from a human or humanized antibody.

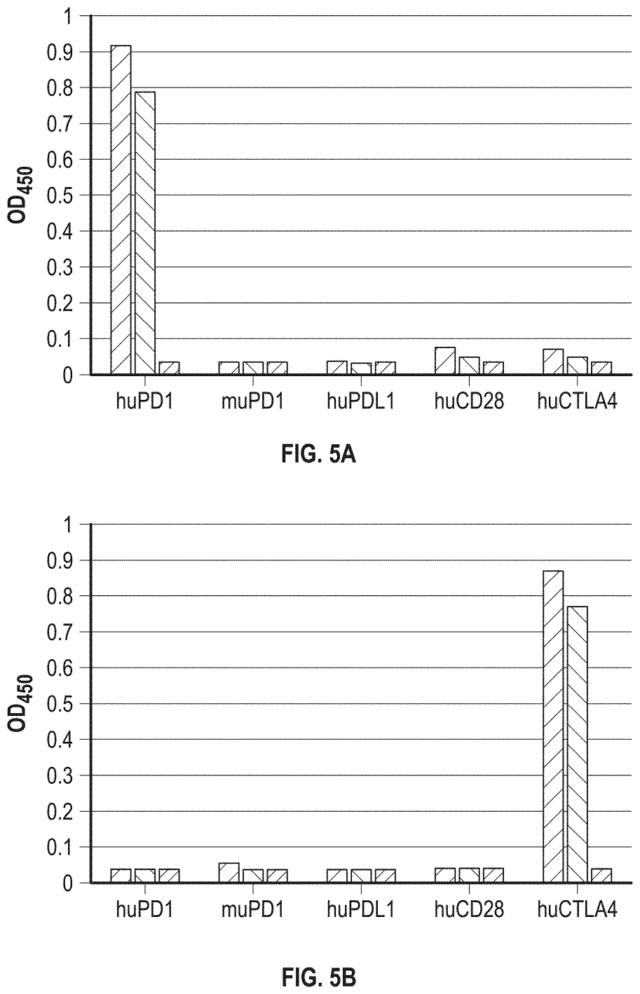

In addition, an anti-hPD1 antibody as described herein can have various functional attributes. In one aspect such anti-hPD1 antibodies can bind to human and cynomolgus monkey PD1, but not to murine PD1, human PDL1, human CD28, or human CTLA4. These functional aspects are demonstrated in Examples 2 and 7 and FIG. 5. Thus, an anti-hPD1 antibody as described herein can exhibit specific binding to human PD1 and the closely related antigen, cynomolgus monkey PD1. Although not every possible related antigen has been tested, the test results provided in Examples 2 and 7 and FIG. 5 define what is meant by “specific” binding to human PD1 as meant herein.

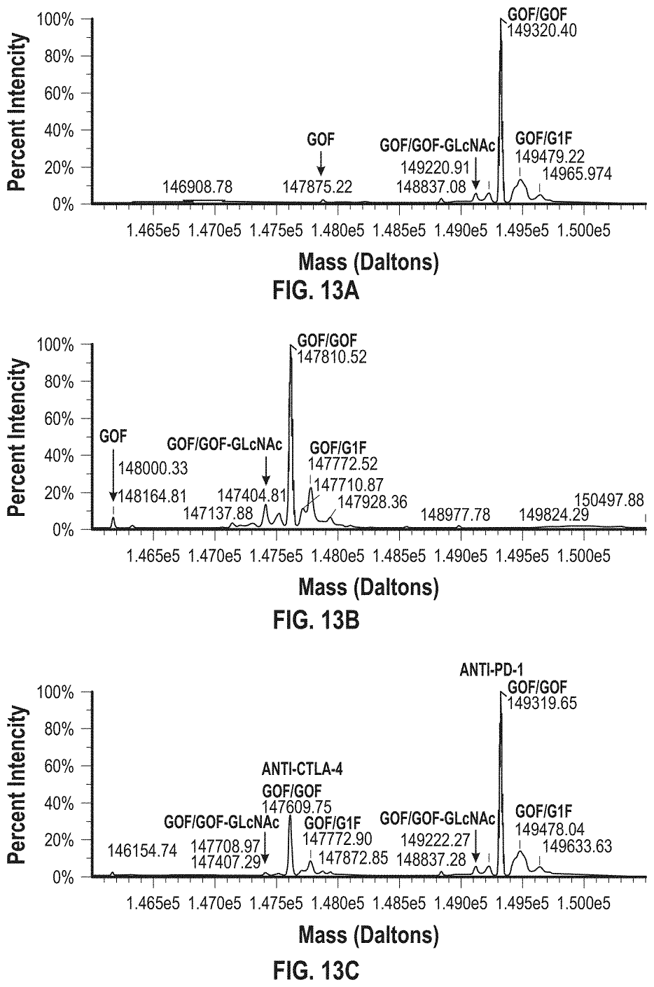

In another aspect, an anti-hPD1 antibody as described herein can block the binding of human PDL1 (hPDL1) to hPD1. This property is demonstrated by data shown in Example 7 and FIGS. 13 and 14 of WO 2018/089293, which are incorporated herein by reference.

In a further aspect, an anti-hPD1 antibody as described herein can bind a monomeric analyte comprising the extracellular domain of human PD1 with a k_{d} of no more than 1×10⁻⁵ l/s, 7×10⁻⁴ l/s, 5×10⁻⁴ l/s, 3×10⁻⁴ l/s, or 2×10⁻⁴ l/s and/or a K_{D} of no more than 30 nM, 20 nM, 10 nM, 7 nM, 5 nM or 4 nM. In another aspect, an anti-hPD1 antibody as described herein can bind a monomeric analyte comprising the extracellular domain of cynomolgus monkey PD1 with a k_{d} of no more than 2×10⁻⁵ l/s, 1×10⁻⁵ l/s, 9×10⁻⁴ l/s, 8×10⁻⁴ l/s, 7×10⁻⁴ l/s, or 6×10⁻⁴ l/s and/or a K_{D} of no more than 30 nM, 20 nM, 10 nM, 8 nM, 7 nM, or 6 nM. Such kinetic measurements can be determined as described in Example 7 using a Biacore optical biosensor. Such monomeric analytes include, e.g., the extracellular domain of hPD1 or cynomolgus monkey PD1 (cPD1) fused to a histidine tag (his tag) and/or a glutathione S-transferase tag (GST tag). In contrast, the extracellular domain of hPD1 or cPD1 fused to the Fc region of an antibody is not a monomeric analyte as meant herein since it would dimerize.

In still another aspect, an anti-hPD1 antibody as described herein can have an in vivo half-life (t_{1/2}) in a single dose study in cynomolgus monkeys in a range of about 100-400, 120-350, 200-350, 250-350, or 275-350 hours.

Further, an anti-hPD1 antibody as described herein can have an in vivo t_{1/2} of 135-300, 135-275, or 140-250 hours in a human subject who has not been previously dosed with the anti-hPD1 antibody.

Further, an anti-hPD1 antibody as described herein can comprise 228P in the amino acid sequence of its HC. The numbering system used in this discussion is that of Edelman et al. Edelmann et al., supra. Such numbering may not correspond exactly to the numbering of a specific antibody, since there can be some variability in the lengths of various portions of an antibody. For avoidance of doubt, this HC position (228) corresponds to position 227 in SEQ ID NO: 1.

### An Anti-hCTLA4 Antibody

An anti-hCTLA4 antibody as described herein can be a human or humanized IgG antibody. The HC of an anti-hCTLA4 antibody as described herein can be a human or humanized IgG HC, such as an IgG1, IgG2, IgG3, or IgG4 HC. In some embodiments, this HC is an IgG1 HC. In one aspect, this HC can be encoded by the nucleic acid sequence of SEQ ID NO: 14. Exemplary amino acid sequences that are encoded by SEQ ID NO: 14 include SEQ ID NO: 13 and/or SEQ ID NO: 22 or amino acid sequences comprising ten, nine, eight, seven, six, five, four, three, two, or one alteration(s) relative to SEQ ID NO: 13 or SEQ ID NO: 22. An amino acid sequence encoded by SEQ ID NO: 14 can comprise post-translational alterations that alter its sequence relative to, for example, SEQ ID NO: 13. The exact nature of such post-translational modifications can depend on the nature of the host cell in which an antibody is produced. An example of such an amino acid sequence is SEQ ID NO: 22, which reflects actual post-translational modifications found in the HC of an anti-hCTLA4 antibody made in a CHO host cell. See Example 6 below. To be clear, an anti-hCTLA4 antibody comprising an HC having the amino acid sequence of SEQ ID NO: 22 can be produced in host cells, for example CHO cells, containing a nucleic acid encoding an HC comprising the amino acid sequence of SEQ ID NO: 13. In another aspect, the amino acid sequence of an HC of an anti-hCTLA4 antibody as described herein can comprise ten, nine, eight, seven, six, five, four, three, two, one, or zero alteration(s) relative to SEQ ID NO: 13 or SEQ ID NO: 22, regardless of the nucleic acid sequence encoding the amino acid sequence of the HC.

A V_{H} of an anti-hCTLA4 in the mixture can be encoded by a nucleic acid sequence encoding the amino acid sequence of, for example, SEQ ID NO: 15 and/or SEQ ID NO: 21 or a nucleic acid sequence encoding an amino acid sequence comprising four, three, two, or one alteration(s) relative to SEQ ID NO: 15 and/or SEQ ID NO: 21. One such nucleic acid sequence is SEQ ID NO: 16. An amino acid sequence encoded by SEQ ID NO: 16 can comprise post-translational alterations that alter its sequence relative to, for example, SEQ ID NO: 15. The exact nature of such post-translational modifications can depend on the nature of the host cell in which an antibody is produced. An example of such an amino acid sequence is SEQ ID NO: 21, which reflects actual post-translational modifications found in the V_{H} of an anti-hCTLA4 antibody made in a CHO host cell. See Example 6 below. To be clear, an anti-hCTLA4 antibody comprising a V_{H} having the amino acid sequence of SEQ ID NO: 21 can be produced in host cells, for example CHO cells, containing a nucleic acid encoding a V_{H} comprising the amino acid sequence of SEQ ID NO: 15. In another aspect, the amino acid sequence of a V_{H} of an anti-hCTLA4 antibody as described herein can comprise four, three, two, one, or zero alteration(s) relative to SEQ ID NO: 15 or SEQ ID NO: 21, regardless of the nucleic acid sequence encoding the amino acid sequence of the V_{H}.

The LC of an anti-hCTLA4 as described herein can be a human or humanized LC comprising a C_{L}κ or C_{L}λ. In some embodiments, this LC can comprise a C_{L}κ. The LC of an anti-hCTLA4 antibody as described herein can be encoded by a nucleic acid sequence encoding the amino acid sequence of SEQ ID NO: 17 and/or SEQ ID NO: 24 or a nucleic acid sequence encoding an amino acid sequence comprising four, three, two, or one alteration(s) relative to SEQ ID NO: 17. One such nucleic acid sequence is SEQ ID NO: 18. An amino acid sequence encoded by SEQ ID NO: 18 can comprise post-translational alterations that alter its sequence relative to, for example, SEQ ID NO: 17. The exact nature of such post-translational modifications can depend on the nature of the host cell in which an antibody is produced. An example of such an amino acid sequence is SEQ ID NO: 24, which reflects actual post-translational modifications found in the LC of an anti-hCTLA4 antibody made in a CHO host cell. See Example 6 below. To be clear, an anti-hCTLA4 antibody comprising an LC having the amino acid sequence of SEQ ID NO: 24 can be produced by host cells, for example CHO cells, containing a nucleic acid encoding a LC comprising the amino acid sequence of SEQ ID NO: 17. In another aspect, an amino acid sequence of an LC of an anti-hCTLA4 antibody as described herein can comprise four, three, two, one, or zero alteration(s) relative to SEQ ID NO: 17 or SEQ ID NO: 24, regardless of the nucleic acid sequence encoding the amino acid sequence of the LC.

A V_{L} of an anti-hCTLA4 antibody as described herein can be encoded by a nucleic acid sequence encoding the amino acid sequence of SEQ ID NO: 19 and/or SEQ ID NO: 23 or a nucleic acid sequence encoding an amino acid sequence comprising four, three, two, or one alteration(s) relative to SEQ ID NO: 19. One such nucleic acid sequence is SEQ ID NO: 20. An amino acid sequence encoded by SEQ ID NO: 20 can comprise post-translational alterations that alter its sequence relative to, for example, SEQ ID NO: 19. The exact nature of such post-translational modifications can depend on the nature of the host cell in which an antibody is produced. An example of such an amino acid sequence is SEQ ID NO: 23, which reflects actual post-translational modifications found in the V_{L} of an anti-hCTLA4 antibody made in a CHO host cell. See Example 6 below. To be clear, an anti-hCTLA4 antibody comprising a V_{L} having the amino acid sequence of SEQ ID NO: 23 can be produced in host cells, for example CHO cells, containing a nucleic acid encoding a V_{L} comprising the amino acid sequence of SEQ ID NO: 19. In another aspect, an amino acid sequence of a V_{L} of an anti-hCTLA4 antibody as described herein can comprise four, three, two, one, or zero alteration(s) relative to SEQ ID NO: 19 or SEQ ID NO: 23, regardless of the nucleic acid sequence encoding the amino acid sequence of the V_{H}.

In addition, an anti-hCTLA4 antibody as described herein has various functional attributes. In one aspect such anti-hCTLA4 antibodies can bind to human and cynomolgus monkey CTLA4, but not to human PD1, murine PD1, human PDL1, or human CD28. These functional aspects are demonstrated in Examples 2 and 7 and FIG. 5. Thus, an anti-hCTLA4 antibody as described herein can exhibit specific binding to human CTLA4 and the closely related antigen, cynomolgus monkey CTLA4. Although not every possible related antigen has been tested, the test results provided in Examples 2 and 7 and FIG. 5 define what is meant by “specific” binding to human CTLA4 as meant herein.

In another aspect, an anti-hCTLA4 antibody as described herein can block the binding of hCTLA4 to its ligands human B7-1 and/or B7-1 (hB7-1 and/or hB7-2) and can block the functional effect of CTLA4 on a target cell. These attributes are demonstrated by data shown in Example 4 of WO 2018/089293, which is incorporated herein by reference.

In a further aspect, an anti-hCTLA4 antibody as described herein can bind a monomeric analyte comprising the extracellular domain of human CTLA4 with a k_{d} of no more than 5×10⁻³ l/s, 2×10⁻³ l/s, 8×10⁻⁴ l/s, 5×10⁻⁴ l/s, 1×10⁻⁴ l/s, or 8×10⁻⁵ l/s and/or a K_{D} of no more than 30 nM, 20 nM, 10 nM, 7 nM, 5 nM, 4 nM, 3 nM, or 2 nM. In another aspect, an anti-hCTLA4 antibody as described herein can bind a monomeric analyte comprising the extracellular domain of cynomolgus monkey CTLA4 with a k_{d} of no more than 5×10⁻³ l/s, 2×10⁻³ l/s, 8×10′ 1/s, 5×10⁻⁴ l/s, or 4×10⁻⁴ l/s and/or a K_{D} of no more than 30 nM, 20 nM, 10 nM, 7 nM, 5 nM, 4 nM, or 3 nM. Such biokinetic measurements can be determined as described in Example 7 using a Biacore optical biosensor. Examples of such monomeric analytes include the extracellular domain of hCTLA4 or cCTLA4 fused to a his tag and/or a GST tag. On the other hand, the extracellular domain of hCTLA4 or cCTLA4 fused to the Fc region of an antibody would not be considered to be a monomeric analyte since it would dimerize.

In still another aspect, an anti-hCTLA4 antibody as described herein can have a single dose serum half-life in cynomolgus monkeys in a range of about 25-200, 50-150, or 75-125 hours.

Further, an anti-hCTLA4 antibody as described herein can have an in vivo t_{1/2} of 90-210, 100-195, 100-140, or 140-250 hours in a human subject who has not been previously dosed with the anti-hCTLA4 antibody.

Further, an anti-hCTLA4 antibody as described herein can comprise specific amino acids at specific sites in its constant domains. These can include one or more of (or all of) the following: 147D in the HC; 170C in the HC; 173C in the HC; 220G in the HC; 255K in the HC; 399R in HC; 409E in the HC; 131K in the LC; 160C in the LC; 162C in the LC; and 214S in the LC. In this discussion, the numbering system of Edelman et al., supra is used. As mentioned above, this numbering may not correspond exactly to the actual position in the amino acid sequence of a specific antibody due to variability in the lengths of various portions of antibodies. For avoidance of doubt, these HC positions correspond (in the same order as above) to the following positions in SEQ ID NO: 13: positions 148, 171, 174, 221, 256, 400, and 410. These LC positions correspond (in the same order as above) to the following positions in SEQ ID NO: 17: positions 131, 160, 162, and 214. An anti-hCTLA4 antibody as described herein can include variable domains encoded by SEQ ID NOs: 16 and 20 and constant domains whose sequences are not included in SEQ ID NOs: 13, 17, 22, and/or 24, with the proviso that these constant domains contain one or more of (or all of) the specific amino acids at the specific positions mentioned in this paragraph. Such antibodies can be IgG1, IgG2, IgG3, or IgG4 antibodies having kappa or lambda LCs and can be human or non-human antibodies.

### An Antibody Mixture

Described herein is a mixture of antibodies comprising two major species of antibodies including an anti-hPD1 antibody and an anti-hCTLA4 antibody, which are described above. A mixture comprising these two antibodies is referred to herein as PSB205. In some embodiments, PSB205 comprises no major species of antibody other than these two major species of antibodies. In some embodiments such a mixture can be made in a host cell containing nucleic acids encoding the anti-hPD1 and anti-hCTLA4 antibodies. In some embodiments, these host cells can produce a mixture that contains no major species of antibodies other than the anti-hPD1 and anti-hCTLA4 antibodies. Thus, separation of these two antibody species from other antibody species that may potentially be produced by the host cells can be unnecessary. Alternatively, the anti-hPD1 and anti-hCTLA4 antibodies in PSB205 can be produced in separate host cell lines and combined in a desired ratio to make the mixture PSB205. PSB205 has particular properties as described below and exemplified in the Examples below.

In one aspect, PSB205 can comprise an anti-hCTLA4 antibody and an anti-hPD1 antibody as described herein in a weight/weight (w/w) ratio (anti-hCTLA4: anti-hPD1 ratio) from about 3:1 to about 1:4, from about 2:1 to 1:4, from about 1:1 to about 1:3, from about 1:1.5 to about 1:2.5, or from about 1:1.7 to about 1:2.3, respectively. In some embodiments, this anti-hCTLA4:anti-hPD1 ratio can be, respectively, about 3:1, 2:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, or 1:4. Thus, PSB205 can comprise a greater quantity of the anti-hPD1 antibody as compared to the quantity of the anti-hCTLA4 antibody or, alternatively, a greater quantity of the anti-hCTLA4 antibody as compared to the quantity of the anti-hPD1 antibody. This ratio, coupled with properties of the anti-hCTLA4 and anti-hPD1 antibodies in PSB205 such as their binding and pharmacokinetic properties, can affect functional properties of PSB205.

In one aspect, the in vivo t_{1/2} in a single dose study in cynomolgus monkeys of the anti-hPD1 antibody that is part of PSB205 is longer than that of the anti-hCTLA4 antibody in PSB205. The ratio of the in vivo t_{1/2} in a single dose study in cynomolgus monkeys of the anti-hCTLA4 antibody compared to that of the anti-hPD1 antibody (t_{1/2}(CTLA4):t_{1/2}(PD1)) can be, respectively, from about 1:4 to about 1:1. More specifically, this ratio can be about 1:4, 1:3.8, 1:3.7, 1:3.6, 1:3.5, 1:3.4, 1:3.3, 1:3.2, 1:3.1, 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6, 1:1.5, 1:1.4, 1:1.3, 1:1.2, 1:1.1, or 1:1. These differing t_{1/2}s, along with the w/w ratio of the two antibodies in PSB205, can ensure that the administration of the antibody mixture PSB205 delivers a dose of the anti-hPD1 antibody that is higher than and/or more slowly cleared in vivo than the dose of the anti-hCTLA4 antibody.

Similarly, in human patients who have not previously been dosed with either the anti-hCTLA4 antibody or the anti-hPD1 antibody, the t_{1/2}(CTLA4):t_{1/2}(PD1) can be about 1:3, 1:2.75, 1:2.5, 1:2.25, 1:2, 1:1.75, 1:1.5 or 1:1.25.

In more specificity, an anti-hPD1 antibody as described herein that is included in PSB205 can have an in vivo t_{1/2} in a single dose study in cynomolgus monkeys from about 150 hours to about 350 hours. In some embodiments, such a t_{1/2} can be from about 275 hours to about 350 hours, from about 280 hours to about 340 hours, from about 290 hours to about 330 hours, or from about 290 hours to about 310 hours. In some embodiments, such a t_{1/2} can be about 292, 293, 294, 295, 296, 297, 298, 299, 300, 301, or 302 hours.

In another aspect, in human patients who have not previously been dosed with an anti-hPD1 antibody as described herein, a t_{1/2} of an anti-hPD1 antibody as described herein that is part of a PSB205 antibody mixture can be from about 120 to about 300 hours, from about 135 to about 300 hours, or from about 140 to about 250 hours. In some embodiments, such a t_{1/2} can be about 135, 140, 145, 147, 150, 155, 160, 165, 170, 175, 180, 185, 190, 195, 200, 205, 210, 215, 220, 225, 227, 230, 240, 250, or 275 hours.

An anti-hCTLA4 antibody as described herein that is included in PSB205 can have an in vivo t_{1/2} from about 30 hours to about 130 hours in a single dose study in cynomolgus monkey. In some embodiments, such a t_{1/2} can be from about 40 hours to about 200 hours, about 40 hours to about 150 hours, about 70 hours to about 130 hours, from about 50 hours to about 120 hours, from about 60 hours to about 110 hours, or from about 80 hours to about 110 hours. In some embodiments, such a t_{1/2} can be about 40, 45, 50, 55, 60, 65, 70, 75, 80, 85, 90, 95, 100, 101, 102, 103, 104, 105, 106, 107, 108, 109, 110, 111, 112, 113, 114, 115, 116, 117, 118, 119, or 120 hours.

In further aspect, in human patients who have not previously been dosed with an anti-hCTLA4 as described herein, a t_{1/2} of an anti-hCTLA4 antibody as described herein that is part of a PSB205 antibody mixture can be from about 80 to about 250 hours, from about 90 to about 210 hours, from about 100 to about 195 hours, or from about 90 to about 140 hours. In some embodiments, such a t_{1/2} can be about 90, 95, 100, 105, 110, 115, 120, 125, 130, 135, 140, 150, 160, 170, 180, or 190 hours.

In another aspect, PSB205 can exhibit synergistic functional effects in vivo as compared to either the anti-hPD1 antibody PSB103 or the anti-hCTLA4 antibody in PSB105. For example, PSB205 can reduce tumor volume and/or tumor diameter in a tumor model system, such as a murine xenograft model system of a human tumor, to a greater extent than either the anti-hPD1 antibody PSB103 or the anti-hCTLA4 antibody PSB105 alone. See, e.g., Example 9. In a further aspect, PSB205 can increase numbers of cytomegalovirus (CMV) specific T cells produced in a CMV recall response assay more than either the anti-hPD1 antibody or the anti-hCTLA4 antibody alone can. Example 8. In another aspect, administration of PSB205 to human cancer patients can lead to a partial response (PR) or a complete response (CR) as defined herein. In a further aspect, administration of PSB205 to human cancer patients can lead to longer progression free survival than administration of a placebo. Further, administration of PSB205 to human cancer patients can lead to a longer progression free survival than administration of either the anti-hPD1 antibody or the anti-hCTLA4 antibody described herein alone.

In a further aspect, administration of PSB205 can produce low levels of adverse events (AEs) in human patients. As defined above and in standard publications in the art (see, e.g., https://ctep.cancer.gov/protocoldevelopment/electronic_applications/docs/ctcae_v5_quick_reference_5x7.pdf) grade 3 or 4 AEs are serious events indicating a need for intervention. Of course, occurrence of AEs can be related to the dose of a drug. In one aspect, a dose of no more than about 0.3 mg/kg or no more than about 24, 21, 18, 15, or 12 mg of PSB205 can produce no grade 3 or 4 AEs. In another aspect, a dose of no more than about 1.0 mg/kg or no more than about 90, 80, 70, 60, 50, or 40 mg of PSB205 can produce no grade 3 or 4 AEs. In still another aspect, a dose of no more than about 3.0 mg/kg or no more than about 270, 240, 210, 180, 150, or 120 mg of PSB205 can produce a grade 3 or 4 AE in no more than ten, nine, eight, seven, six, five, four, three, two, or one percent of patients dosed or, in some embodiments, in none of the patients dosed. In a further aspect, a dose of no more than about 5.0 mg/kg or no more than about 450, 425, 400, 375, 350, 325, 300, 275, 250, 225, or 200 mg of PSB205 can produce a grade 3 or 4 AE in no more than 15, 14, 13, 12, 11, ten, nine, eight, seven, six, five, four, three, two, or one percent of patients dosed or, in some embodiments, in none of the patients dosed.

### Polynucleotides Encoding an Anti-hPD1 and/or an Anti-hCTLA4 Antibody

Nucleic acids encoding an anti-hPD1 or an anti-hCTLA4 antibody as described herein or a mixture containing both antibodies, i.e., PSB205, can be made as described below in Examples 1 and 4 or by using other appropriate methods using the sequences and other disclosure provided herein. For example, given the disclosure herein, DNA sequences encoding the anti-hPD1 and anti-hCTLA4 antibodies described herein could be synthesized. In another aspect, vectors encoding the HC and LC from an anti-hPD1 antibody and from an anti-hCTLA4 antibody as described herein could be made as described in Example 1.

Polynucleotides comprising specific nucleotide sequences encoding the HC, LC, V_{H}, and V_{L} of an anti-hPD1 antibody described herein include, respectively, polynucleotides comprising SEQ ID NO: 2, SEQ ID NO: 6, SEQ ID NO: 4, and SEQ ID NO: 8. These sequences encode, respectively, the following amino acid sequences: SEQ ID NO: 1, SEQ ID NO: 5, SEQ ID NO: 3, and SEQ ID NO: 7. Because of the degeneracy of the genetic code, other nucleotide sequences can also encode SEQ ID NO: 1, SEQ ID NO: 5, SEQ ID NO: 3, and SEQ ID NO: 7. Polynucleotides comprising such nucleotide sequences are also within the ambit of polynucleotides contemplated herein. Polynucleotides comprising nucleotide sequences encoding amino acid sequences having ten, nine, eight, seven, six, five, four, three, two or one alteration(s) relative to SEQ ID NO: 1 are also contemplated, as are polynucleotides comprising nucleotide sequences encoding amino acid sequences having four, three, two, or one alteration(s) relative to SEQ ID NO: 5, SEQ ID NO: 3, and/or SEQ ID NO: 7.

Polynucleotides comprising specific nucleotide sequences encoding the HC, LC, V_{H}, and V_{L} of an anti-hCTLA4 antibody described herein include, respectively, polynucleotides comprising SEQ ID NO: 14, SEQ ID NO: 18, SEQ ID NO: 16, and SEQ ID NO: 20. These sequences encode, respectively, the following amino acid sequences: SEQ ID NO: 13, SEQ ID NO: 17, SEQ ID NO: 15, and SEQ ID NO: 19. Because of the degeneracy of the genetic code, other nucleotide sequences can also encode SEQ ID NO: 13, SEQ ID NO: 17, SEQ ID NO: 15, and SEQ ID NO: 19. Polynucleotides comprising such nucleotide sequences are also within the ambit of polynucleotides contemplated herein. Polynucleotides comprising nucleotide sequences encoding amino acid sequences having ten, nine, eight, seven, six, five, four, three, two or one alteration(s) relative to SEQ ID NO: 13 are also contemplated, as are polynucleotides comprising nucleotide sequences encoding amino acid sequences having four, three, two, or one alteration(s) relative to SEQ ID NO: 17, SEQ ID NO: 15, and/or SEQ ID NO: 19.

A vector or vectors comprising (a) polynucleotide(s) encoding an anti-hPD1 and/or anti-hCTLA4 antibody as described herein can made in be any of a variety of kinds of vectors. The vector can include a selectable marker for selection of host cells containing the vector and/or for maintenance and/or amplification of the vector in the host cell. Such markers include, for example, (1) genes that confer resistance to antibiotics or other toxins, e.g., ampicillin, tetracycline, or kanamycin for prokaryotic host cells, (2) genes that complement auxotrophic deficiencies of the cell, or (3) genes whose operation supplies critical nutrients not available from complex or defined media. Specific selectable markers include, for example, the kanamycin resistance gene, the ampicillin resistance gene, and the tetracycline resistance gene. A zeocin resistance or neomycin resistance gene may also be used for selection in both prokaryotic and eukaryotic host cells. A dihydrofolate reductase (DHFR) gene and/or a promoterless thymidine kinase gene can be used in mammalian cells, as is known in the art. See, e.g., Kingston et al. 2002, Amplification using CHO cell expression vectors, Current Protocols in Molecular Biology, Ch. 16, Unit 16.23, Wiley 2002.

In addition, a vector can contain one or more other sequence elements necessary for the maintenance of the vector and/or the expression of the inserted sequences encoding the antibodies or antibody mixtures described herein. Such elements include, for example, an origin of replication, a promoter, one or more enhancers, a transcriptional terminator, a ribosome binding site, a polyadenylation site, a polylinker insertion site for exogenous sequences (such as the DNA encoding an antibody or mixture of antibodies described herein), and an intervening sequence between two inserted sequences, e.g., DNAs encoding an HC and an LC. These sequence elements can be chosen to function in the desired host cells so as to promote replication and/or amplification of the vector and expression and of the heterologous sequences inserted into the vector. Such sequence elements are well known in the art and available in a large array of commercially available vectors.

In some embodiments, the polynucleotides encoding an anti-hCTLA4 or an anti-hPD1 antibody as described herein or a mixture of these antibodies described herein, i.e., PSB205, can be carried on one or more viral vector, optionally an oncolytic viral vector. Examples of such viral vectors include adenovirus, adeno-associated virus (AAV), retrovirus, vaccinia virus, modified vaccinia virus Ankara (MVA), herpes virus, lentivirus, Newcastle Disease virus, measles virus, coxsackievirus, reovirus, and poxvirus vectors. In such embodiments, these viral vectors containing polynucleotides encoding the antibody or mixture of antibodies described herein can be administered to patients to treat a disease.

In a cancer patient, for example, such viral vectors containing polynucleotides encoding an antibody or mixture of antibodies can be administered directly to a tumor or a major site of cancer cells in the patient, for example by injection, inhalation (for, e.g., a lung cancer), topical administration (for, e.g., a skin cancer), and/or administration to mucus membrane (through which the nucleic acids can be absorbed), among many possibilities. Alternatively, such viral vectors can be administered systemically, for example, orally, topically, via a mucus membrane, or by subcutaneous, intravenous, intraarterial, intramuscular, or peritoneal injection as described herein.

Similarly, polynucleotides encoding an anti-hCTLA4 or an anti-hPD1 antibody or a mixture of these antibodies as described herein can be encased in carrier structure, e.g., liposomes, which can be administered to a patient suffering from a disease. Polynucleotides contemplated herein include RNA and DNA, as well as chemically modified polynucleotides that are, for example, more stable and/or efficacious than naturally-occurring DNA and/or RNA. See, e.g., Burnett and Rossi (2012), RNA-based therapeutics—current progress and future prospects, Chem. Biol. 19(21): 60-71. These encased polynucleotides can be administered directly to a tumor or a major site of cancer cells in the patient, for example by injection, inhalation (for, e.g., a lung cancer), topical administration (for, e.g., a skin cancer), and/or administration to mucus membrane (through which the nucleic acids can be absorbed), among many possibilities. Alternatively, such encased polynucleotides can be administered systemically, for example, orally, topically, via a mucus membrane, or by subcutaneous, intravenous, intraarterial, intramuscular, or peritoneal injection as described herein.

### Pharmaceutical Compositions

The antibodies, antibody mixtures, polynucleotides, and/or vectors described herein can be administered in a pharmaceutically acceptable formulation. With regard to the mixtures of antibodies, each antibody can be formulated and administered either separately or together. Numerous pharmaceutical formulations are known in the art. Many such formulations are described in REMINGTON: THE SCIENCE AND PRACTICE OF PHARMACY, 21^{st} ed., Lippincott Williams & Wilkins, Philadelphia, Pa., 2005, the relevant portions of which are incorporated herein by reference. Such a pharmaceutically acceptable formulation can be, for example, a liquid such as a solution or a suspension, a solid such as a pill, a capsule, a paste, or a gel. A liquid formulation can contain, for example, one or more of the following components: a buffer, an excipient, a salt, a sugar, a detergent, and a chelating agent. It can be designed to preserve the function of the antibody, antibody mixture, polynucleotide, or vector and to be well tolerated by the patient.

For an antibody or a mixture of antibodies, a pharmaceutical composition can have a pH from about 4.5 to about 7.5, from about 4.5 to about 7.0, from about 4.5 to about 6.5, from about 4.5 to about 6.0, or from 4.5 to about 5.5. The concentration of antibody in such a formulation can be from about 5 mg/mL to about 40 mg/mL, from about 10 mg/mL to about 35 mg/mL, from about 15 mg/mL to about 30 mg/mL, or from about 20 mg/mL to about 30 mg/mL. The osmolality of such a composition can range from about 250 mOsm/kg to about 380 mOsm/kg, from about 260 mOsm/kg to about 350 mOsm/kg, from about 275 to about 295 mOsm/kg, and/or from about 280 mOsm/kg to about 290 mOsm/kg. Such compositions can comprise a sugar, such as sucrose, trehalose, or sorbital, among many other possibilities. Such compositions can comprise a salt, for example, a sodium salt, a hydrochloride salt, a sulfate salt, an acetate salt, or a phosphate salt, among many possibilities. Such composition can comprise a surfactant such as polysorbate-20, among other possibilities.

Polynucleotides and proteins such as antibodies are usually administered parenterally, as opposed to orally. Depending on the formulation, oral administration could subject the protein or polynucleotide to the acidic environment of the stomach, which could inactivate the protein or polynucleotide, for example, by hydrolyzing a protein. In some embodiments, a specific formulation might allow oral administration of a specific protein or polynucleotide where the protein or polynucleotide is either insensitive to stomach acid or is adequately protected from the acidic environment, e.g., by a specific coating on a pill or capsule. A formulation could also be administered via a mucus membrane, including, for example, intranasal, vaginal, rectal, or oral administration, or administration as an inhalant. A formulation could also be administered topically in some embodiments. Commonly, antibodies and polynucleotides are administered by parenteral injection of a liquid formulation, for example, by subcutaneous, intravenous, intraarterial, intralesional (e.g., intratumoral), intramuscular, or peritoneal injection.

Targeted inhibitors, which are small molecules, can be administered orally or by other methods as described above. Appropriate formulations for oral administration can include, for example, a liquid, such as a solution or a suspension, a paste, a gel, a capsule, or a solid, such as a pill.

### Host Cells Containing Nucleic Acids Encoding Anti-hCTLA4 and/or Anti-hPD1 Antibodies

Nucleic acids encoding an anti-hCTLA4 or anti-hPD1 antibody or a mixture thereof as described herein or vectors carrying such nucleic acids as can be introduced (e.g., by transfection, transduction, lipofection, transformation, bombardment with microprojectiles, microinjection, or electroporation) into host cells individually, at the same time, or sequentially. In some embodiments, they could be introduced sequentially as described in Example 4. Such host cells containing nucleic acids encoding an anti-hPD1 and/or an anti-hCTLA4 antibody as described herein of can contain nucleic acids encoding both an anti-hPD1 and an anti-hCTLA4 antibody as described herein.

These host cells can be mammalian, protozoan, fungal, plant, or bacterial cells. More specifically, gram negative or gram positive prokaryotes, for example, bacteria such as Escherichia coli, Bacillus subtilis, or Salmonella typhimurium can be used as host cells. In other embodiments, a host cell can be a eukaryotic cell, including such species as Saccharomyces cerevisiae, Schizosaccharomyces pombe, or eukaryotes of the genus Kluyveromyces, Candida, Spodotera, or any cell capable of expressing heterologous polypeptides.

In further embodiments, a host cell can be a mammalian cell. Many mammalian cell lines suitable for expression of heterologous polypeptides are known in the art and can be obtained from a variety of vendors including, e.g., American Type Culture Collection (ATCC). Suitable mammalian host cell lines include, for example, the COS-7 line (ATCC CRL 1651) (Gluzman et al., 1981, Cell 23:175), L cells, C127 cells, 3T3 cells (ATCC CCL 163), Chinese hamster ovary (CHO) cells, or their derivatives such as Veggie CHO and related cell lines, which grow in serum-free media (Rasmussen et al., 1998, Cytotechnology 28: 31), CHO-K1 and CHO pro-3 cell lines and their derivatives such as the DUKX-X11 and DG44 cell lines, which are deficient in dihydrofolate reductase (DHFR) activity, HeLa cells, baby hamster kidney (BHK) cells (e.g., ATCC CRL 10), the CVI/EBNA cell line derived from the African green monkey kidney cell line CVI (ATCC CCL 70) as described by McMahan et al., 1991, EMBO J. 10: 2821, human embryonic kidney (HEK) cells such as 293, 293 EBNA or MSR 293, human epidermal A431 cells, human Colo205 cells, HL-60 cells, U937 cells, HaK cells, Jurkat cells, HepG2/3B cells, KB cells, NIH 3T3 cells, S49 cells, and mouse myeloma cells, including NS0 and Sp2/0 cells. Other prokaryotic, eukaryotic, or mammalian cell types that are capable of expression of a heterologous polypeptide could also be used.

In more detail, a host cell line, e.g., a CHO cell line, containing nucleic acids encoding a mixture of an anti-hCTLA4 antibody and an anti-hPD1 antibody as described herein, i.e., PSB205, can produce these two antibodies in a stable ratio that is, e.g., about 1:2 (anti-hCTLA4:anti-hPD1). In some embodiments, an anti-hCTLA4:anti-hPD1 ratio of antibodies produced by host cells can be from about 3:1 to about 1:4, from about 2:1 to 1:4, from about 1:1 to about 1:3, from about 1:1.5 to about 1:2.5, or from about 1:1.7 to about 1:2.3. In further embodiments, an anti-hCTLA4:anti-hPD1 ratio in an antibody mixture produced by host cells can be about 3:1, 2:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, or 1:4. In further embodiments, an anti-hCTLA4:anti-hPD1 ratio produced by host cells can be from about 1:1.8 to about 2.2. Such ratios can be maintained for cells at a population doubling level (PDL) of least about 30, 35, 40, 45, 50, 60, 70, 80, 90, or 100.

In a further aspect, a host cell, e.g., a CHO cell line, as described herein can produce a total amount of antibody in its cell supernatant of at least about 0.6, 0.7, 0.8, 0.9. 1.0, 1.1, 1.2, 1.3, 1.4, 1.5, 2, 2.5, or 3 grams/liter (g/L). In other embodiments, antibody production by a host cell can be within one or more of the following ranges: 1.0-4.0 g/L, 1.0-3.0 g/L, 1.0-2.0 g/L, 2.0-4.0 g/L, 0.5-2.0 g/L, 0.5-1.5 g/L, 0.6-1.4 g/L, 0.7-1.3 g/L, 0.8-1.2 g/L, or 0.9-1.2 g/L.

In another aspect, the cell doubling time of a CHO host cell line that can produce PSB205 can be from about 18-32, 18-30, 19-28, or 20-25 hours. Further, such a host cell line can maintain its doubling time within this range at a population doubling level (PDL) of from 10-200, 20-200, 10-175, 20-150, 20-100, 20-60, or 10-60. A PDL, as meant herein, is the number of times the cells in a population have doubled since their thawing from a frozen cell bank.

### Methods of Making the Antibodies or the Antibody Mixture

The antibodies and mixtures of antibodies described herein can be made by various methods. An anti-hPD1 and/or anti-hCTLA4 antibody as described herein can be made by introducing (a) polynucleotide(s) encoding the antibody or antibodies into a host cell, culturing the host cell, recovering the antibody or antibodies from the cell mass or cell supernatant, and, optionally, purifying the antibody.

In some embodiments, an antibody mixture as described herein can be made in two separate host cell lines, one of which produces an anti-hPD1 antibody and one of which produces an anti-hCTLA4 antibody. In such an embodiment, the two host cell lines are cultured separately or together, and the antibody or antibodies that they produce can be purified from the cell supernatant(s) or the cell mass(es). In some embodiments, host cell lines are cultured separately, and the cell supernatants or cell masses are combined prior to purification of the antibodies. In other embodiments, the two cell lines are cultured separately, and the antibodies are purified separately from the two cell supernatants or cell masses. In further embodiments the two host cell lines are cultured together, and the antibodies are purified from the cell supernatant or the cell mass. When the two cell lines are cultured separately, the antibodies can be combined in any desired ratio. Purification can be done as needed, including potential steps such as, for example, Protein A column chromatography, anion or cation exchange chromatography, size exclusion chromatography, hydrophobic interaction chromatography, various purification by precipitation strategies, etc.

In another embodiment, an antibody mixture as described herein can be made in a single host cell line, e.g., a clonal CHO cell line as described in Example 4, that contains polynucleotides encoding both the anti-hPD1 and the anti-hCTLA4 antibodies. In this case, the host cell line is cultured, and the antibody produced by the host cells is isolated from the cell supernatant or the cell mass. A host cell line that produces an antibody mixture as described herein produces at most three or two major species of antibodies. Further purification can be done as needed, including potential steps such as, for example, Protein A column chromatography, anion or cation exchange chromatography, size exclusion chromatography, hydrophobic interaction chromatography, various purification by precipitation strategies, etc. In some embodiments, the host cell line produces only two major species of antibodies, that is anti-hPD1 antibody PSB103 and anti-hCTLA4 antibody PSB105 as described herein. In such a case, it may be unnecessary to purify these species from other antibody species that could in some situations be present among antibodies species produced by the host cells. In such a situation, the antibody mixture PSB205 could be produced with a single production and purification process.

### Therapeutic Methods

An anti-hCTLA4 or anti-hPD1 antibody or a mixture thereof, i.e., PSB205, or (a) polynucleotide(s) or (a) vector(s) encoding any of these therapeutics can be administered to human patients to treat a variety of conditions. Optionally, such therapies can be administered parenterally, although oral routes may be possible if the therapeutic is formulated specifically to make oral administration possible without destruction of the therapeutic in the acid environment of the stomach. In some embodiments, such therapeutics can be administered by injection, optionally, for example, by intramuscular, subcutaneous, intravenous, intraarterial, intradermal, or intratumoral injection. Injections can be administered by infusion or in a bolus. In some embodiments, administration of the therapeutic can occur through a mucosal membrane. Such routes of administration include, e.g., nasal, rectal, or vaginal administration or administration under the eyelids or the tongue (without swallowing) or via inhalation.

A dose PSB205 can be at least 0.1 mg/kg and not more than 5, 10, or 15 mg/kg. In some embodiments, the dosage can be less than or equal to 10, 8, 5, 3, 2, or 1 mg/kg and/or at least 0.1, 0.3, 1, 2, or 3 mg/kg. In some embodiments, the dosage can be about 0.1 mg/kg, 0.3 mg/kg, 1.0 mg/kg, 2.0 mg/kg, 3.0 mg/kg, 4.0 mg/kg, 5.0 mg/kg, 6 mg/kg, 7 mg/kg, 8 mg/kg, or 9 mg/kg. Further a dosage may be defined as a specific amount, independent of the weight of the patient. Such doses can range from about 5 mg to about 800 mg. In particular embodiments, such a dose can be no more than 800, 700, 600, 550, 500, 475, 450, 425, 400, 375, 350, 325, 300, 275, 250, 225, 200, 175, 150, 100, 75, or 50 mg and/or at least about 60, 80, 100, 150, 200, 250, or 300 mg. Further, a dose can be about 125, 150, 175, 200, 225, 250, 275, 300, 325, 350, 375, 400, 425, or 450 mg. Alternatively, a dosage can be defined relative to the surface area of the skin of a patient. For example, in some embodiments a dosage can be at least 3.5 mg/mm² and not more than 180 mg/mm². In some embodiments, a dose can be no more than 400, 350, 300, 350, 200, 180, 150, 110, 75, 50, 40, 30, 25, 12, 10, 7.5, or 5 mg/mm² and/or at least 0.2, 0.5, 1, 3, 5, 10, 20, 30, 50, 75, or 100 mg/mm².

Doses of an anti-hCTLA4 or anti-hPD1 antibody can be at least 0.033 mg/kg and not more than 3.35, 6.7, or 10 mg/kg. In some embodiments, the dosage can be less than or equal to 10, 6.7, 4.8, 3.35, 2, or 0.67 mg/kg and/or at least 0.033, 0.1, 0.33, 0.67, or 1 mg/kg. In some embodiments, the dosage can be about 0.033 mg/kg, 0.1 mg/kg, 0.67 mg/kg, 1.0 mg/kg, 1.67 mg/kg, 3.0 mg/kg, 3.33 mg/kg, 4.0 mg/kg, 5.0 mg/kg, 6 mg/kg, 7 mg/kg, 8 mg/kg, or 10 mg/kg. Further a dosage may be defined as a specific amount, independent of the weight of the patient. Such doses can range from about 60 mg to about 700 mg. In particular embodiments, such a dose can be no more than 700, 600, 500, 450, 400, 350, 300, 250, 215, 170, 130, 100, or 70 mg and/or at least about 0.033, 0.1, 0.7, 1.5, 2, 2.7, 3.5, 5, 7, 10, 15, 17, 20, 25, 35, 45, 55, 65, 100, or 150 mg. Alternatively, a dosage can be defined relative to the surface area of the skin of a patient. For example, a dosage can be at least 0.1, 0.5, or 1.1 mg/mm² and not more than 350, 300, 250, 200, or 130 mg/mm². In some embodiments, a dose can be no more than 300, 200, 130, 120, 100, 75, 50, 35, 30, 20, 15, 10, 7.5, 5, or 3 mg/mm² and/or at least 0.18, 0.3, 1, 2, 3, 6, 10, 17, 25, 33, 66, or 80 mg/mm².

A dose of (a) polynucleotide(s) encoding PSB205 or an anti-hCTLA4 or anti-hPD1 antibody alone, or of (a) vector(s) containing such (a) polynucleotide(s), can be at least about 10⁹, 10¹⁰, 10¹¹, 10¹², 10¹³ copies of the polynucleotide(s) or vector(s) per kilogram of patient body weight (copies/kg). In another aspect, such a dose can be at most about 6×10¹⁴, 7×10¹⁴, 8×10¹⁴, 9×10¹⁴, or 10¹⁵ copies/kg. In a further aspect, such a dose can be from about 10¹⁰ copies/kg to about 10¹⁴ copies/kg. Alternatively, doses can be about 10⁹, 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, 10¹⁵, or 10¹⁶ copies of the polynucleotide(s), regardless of patient body weight.

The frequency of dosing in amounts discussed above can be adjusted. In some embodiments an anti-hCTLA4 or anti-hPD1 antibody, PSB205, or (a) polynucleotide(s) encoding any of these therapeutics can be administered once every three weeks. In other embodiments, such therapeutics can be administered twice per week, once per week, once every 10 days, once every two weeks, or once every three, four, five, six, seven, eight, nine, or 10 weeks. In further embodiments, such therapeutics can be administered once every two, three, four, five, six, seven, eight, nine, 10, 11, or 12 months.

An anti-hCTLA4 or anti-hPD1 antibody, PSB205, or (a) polynucleotide(s) encoding any of these can be used to treat human patients having a variety of conditions. Since such therapeutics can enhance some aspects of an immune response, the conditions for which they are a useful generally include conditions where an enhanced immune response is helpful. Whether an immune response has been enhanced by a particular therapeutic, as meant herein, can be assessed by a CMV recall response assay as described in Example 8. The conditions treatable with the above-mentioned therapeutics include infections, immunodeficiency disorders, and various cancers including, without limitation, melanoma, lung cancer, including squamous non-small cell lung cancer and small cell lung cancer, nasopharyngeal cancer, squamous cell carcinoma of the head and neck, gastric or gastroesophageal carcinoma, clear cell or non-clear cell renal cell carcinoma, urothelial cancer, soft tissue or bone sarcoma, mesothelioma, classical Hodgkin lymphoma, primary mediastinal large B-cell lymphoma, bladder cancer, Merkel cell carcinoma, neuroendocrine carcinoma, cervical cancer, hepatocellular carcinoma, ovarian cancer, microsatellite instability high (MSI-H) or DNA mismatch repair deficient (dMMR) adult and pediatric solid tumors, clear cell renal sarcoma, colorectal cancer, esophageal cancer including esophageal squamous cell carcinoma, endometrial cancer, tumor mutational burden-high cancer, and cutaneous squamous cell carcinoma.

In one aspect, treatment with PSB205 can result in the occurrence of some adverse events (AEs) in a percentage of patients, which can depend on the dose administered and/or the frequency of dosing. It can also depend on the presence of other drugs that may be administered concurrently with PSB205. For the purposes of determining AEs associated with PSB205, patients that are concurrently receiving other drugs or treatments known to cause significant AEs, such as, e.g., chemotherapy or radiation, are excluded. In some instances, AEs can include serious AEs such as grade 3 or grade 4 AEs. To determine the percentage of patients that experience a grade 3 or 4 AE at a particular dose and dosing frequency, at least about 10, 20, 30, 40, 50, 60, 70, 80, 90, 100, or more patients can be dosed. In some embodiments, when ten or more patients are dosed with no more than 3 mg/kg PSB205 once every three weeks, no more than 20, 15, ten, nine, eight, seven, six, five, four, three, two, or one percent of the dosed patients experience a grade 3 or grade 4 AE. In other embodiments, none of these patients experience a grade 3 or 4 AE. In embodiments where ten or more patients are dosed with no more than 5 mg/kg PSB205 once every three weeks, no more than 20, 15, ten, nine, eight, seven, six, five, four, three, two, or one percent of the patients experience a grade 3 or grade 4 AE.

In another aspect, treatment with PSB205 can effectively treat a variety of conditions, for example various cancers recited above. To determine a rate of efficacy, for example an objective response rate (ORR) or a disease control rate (DCR), at least about 10, 20, 30, 40, 50, 60, 70, 80, 90, 100, or more patients can be dosed. In some embodiments, when ten or more cancer patients are dosed with at least about 3 mg/kg and no more than 5 mg/kg PSB205 about once every two, three, or four weeks, the ORR can be at least about one, two, three, four, five, ten, 20, 30, 40, 50, or 60 percent. In some embodiments, when ten or more cancer patients are dosed with at least about 3 mg/kg and no more than 5 mg/kg PSB205 about once every two, three, or four weeks, the DCR can be at least about one, two, three, four, five, ten, 20, 30, 40, 50, or 60 percent. In some embodiments, the patient treated can have lung cancer or nasopharyngeal cancer.

An anti-hCTLA4 or anti-hPD1 antibody, PSB205, or (a) polynucleotide(s) encoding any of these can be administered with an additional therapy, which is administered before, after, and/or concurrently with the antibody, mixture of antibodies, or polynucleotide(s). The additional therapy can be selected from the group consisting of immunomodulatory molecules, radiation, a chemotherapeutic agent, a targeted biologic, a targeted inhibitor, and/or an oncolytic virus.

In some embodiments the additional therapy can be an antagonist of PDL1, TIGIT, CCR4, CCR8, CSFR1a, B7H3, B7H4, CD96, or CD73, an agonist of GITR, 41BB, OX40, or CD40, an oncolytic virus such as talimogene laherparepvec (IMLYGIC™), a bispecific T cell engager (BiTE) such as blinatumomab, an indoleamine 2, 3 dioxygenase (IDO) inhibitor, an anti-angiogenic agent such as bevacizumab, an antibody-drug conjugate, or a tyrosine kinase inhibitor.

If the additional therapy is a chemotherapeutic, it can, for example, be busulfan, temozolomide, cyclophosphamide, lomustine (CCNU), streptozotocin, methyllomustine, cis-diamminedichloroplatinum, thiotepa, aziridinyl benzoquinone, cisplatin, carboplatin, melphalan hydrochloride, chlorambucil, ifosfamide, mechlorethamine HCl, carmustine (BCNU)), adriamycin (doxorubicin), daunomycin, mithramycin, daunorubicin, idarubicin, mitomycin C, bleomycin, vincristine, vindesine, vinblastine, vinorelbine, paclitaxel, docetaxel, VP-16, VM-26, methotrexate with or without leucovorin, 5-fluorouracil with or without leucovorin, 5-fluorodeoxyuridine, 5-fluorouracil, 6-mercaptopurine, 6-thioguanine, gemcitabine, cytarabine, 5-azacytidine, hydroxyurea, deoxycoformycin, fludarabine, etoposide, irinotecan, topotecan, actinomycin D, dacarbazine (DTIC), mAMSA, procarbazine, hexamethylmelamine, pentamethylmelamine, L-asparaginase, mitoxantrone. See, e.g., Cancer: Principles and Practice of Oncology, 4.sup.th Edition, DeVita et al., eds., J.B. Lippincott Co., Philadelphia, Pa. (1993), the relevant portions of which are incorporated herein by reference.

Having described the invention in general terms above, the specific Examples below are offered to exemplify the invention, not limit its scope. It is understood that various changes and modifications may be made to the invention that are in keeping with the spirit of the invention described herein and would be apparent to one of skill in the art. Such changes and modifications are within the scope of the invention described herein, including in the appended claims.

### EXAMPLES

### Example 1: Making Individual Anti-hPD1 and Anti-hCTLA4 Antibodies

A single vector comprising sequences encoding the HC and LC of anti-hPD1 antibody PSB103 was created as follows. Sequences of DNA fragments encoding the amino acid sequence of SEQ ID NO: 1 and SEQ ID NO: 5 were optimized for expression in hamster (Cricetulus griseus) cells using GeneOptimizer™ online software (GeneArt, ThermoFisher Scientific). The resulting optimized DNA sequences, i.e., SEQ ID NOs: 2 and 6, were chemically synthesized. The DNA sequences encoding the HC and the LC were separately subcloned into a transient expression vector and introduced into Escherichia coli cells. The HC of this antibody contained the alteration S228P (where position 228 is as shown in Edelmann et al., supra; which corresponds to position 227 in SEQ ID NO: 1). This alteration can prevent Fab arm exchange in IgG4 antibodies. Silva et al. (2015), The S228P mutation prevents in vivo and in vitro IgG4 Fab-arm exchange as demonstrated using a combination of novel quantitative immunoassays and physiological matrix preparation, J. Biol. Chem. 290(9):5462-5469.

The inserts in the plasmid DNAs from the E. coli cells were sequenced to ensure that the sequences were correct. The sequences were 100% identical to the designed sequences. These plasmid DNAs were used as templates to separately amplify the HC- and LC-encoding sequences by polymerase chain reaction (PCR) using primers including AvrII and BstZ17I sites (HC) or EcoRV and PacI sites (LC). The resulting PCR products were purified by cutting bands from an agarose gel and purifying the DNA fragments from these gel bands. The band encoding the HC was digested with AvrII and BstZ17I and ligated into a Freedom® pCHO 1.0 vector (ThermoFisher Scientific) digested with these same enzymes, which was then introduced into E. coli cells. Plasmid DNAs from individual colonies were sequenced, and one colony that contained plasmid DNA including the designed DNA sequence, which encoded the exact amino acid sequence of the HC of PSB103 was identified. This colony was expanded, and its plasmid DNA was purified.

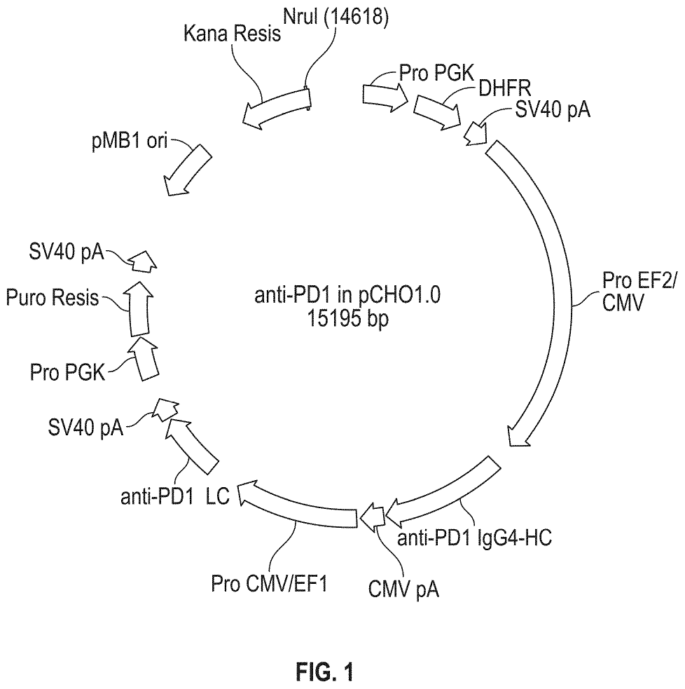

The purified band encoding the LC was digested with EcoRV and PacI and ligated into the Freedom® pCHO 1.0 vector containing inserted DNA encoding the HC of PSB103 described above, which had been digested with the same enzymes. This DNA was introduced into E. coli cells. Individual colonies were selected on kanamycin, and plasmid DNA from selected colonies was sequenced. A colony containing plasmid DNA with inserts that matched the sequences encoding the HC and LC of the anti-hPD1 antibody was re-streaked twice. A single colony was picked, and both strands of its plasmid DNA were sequenced. The sequence matched the vector sequence, and the inserts in the plasmid were 100% matches for the sequences encoding the HC and LC of anti-hPD1 antibody PSB103. FIG. 1 shows a diagram of this vector with its inserts.

As an initial step in producing a clonal cell line producing PSB103, a research cell bank (RCB) of CHO-S™ cells (ThermoFisher Scientific) was made as follows. A Master Cell Bank (MCB) vial of CHO-S™ cells produced under current Good Manufacturing Practices (cGMPs) was thawed in a 37° C. water bath and inoculated into 29 mL of CD FortiCHO™ Medium (ThermoFisher Scientific) supplemented with 8 mM L-glutamine. When a sufficient cell number was obtained, cells were centrifuged, the cell pellet was resuspended in FortiCHO™ Medium supplemented with 8 mM L-glutamine and 10% dimethyl sulfoxide (DMSO) at a concentration of 10⁷ cells/mL, and 1 mL aliquots of these cells were distributed into vials. Vials of cells were frozen, transferred to a −70° C. freezer overnight, and then transferred to the vapor phase of a liquid nitrogen freezer.

Then the Freedom pCHO 1.0 vector containing inserts encoding the HC and LC of anti-hPD1 antibody PSB103 was introduced into CHO cells from the RCB. As described in detail below, a clonal cell line (called G19G4-4B4) stably expressing anti-hPD1 antibody PSB103 was established. G19G4-4B4 was cultured, and PSB103 was recovered from the cell supernatant and purified for use in the experiments described below.

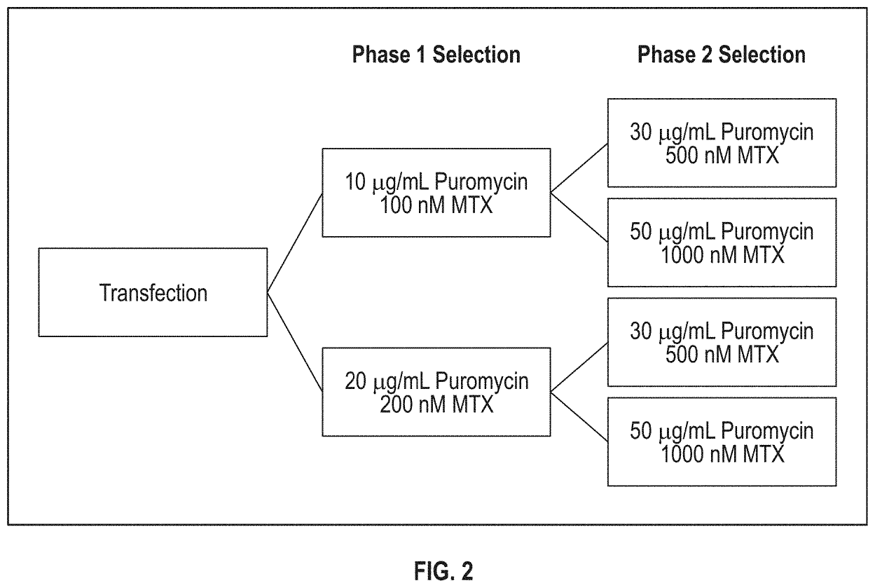

In more detail, the vector encoding the HC and LC of anti-hPD1 antibody PSB103 (which is diagrammed in FIG. 1) was linearized by cleavage with the restriction enzyme NruI, and three parallel transfections of CHO-S™ cells were performed using FreeStyle™ MAX Reagent (ThermoFisher) according to the manufacturer's instructions. See Freedom™ CHO-S™ Kit (catalog number A1369601) User Guide, Publication Number MAN0003505, Revision C.0, ThermoFisher Scientific, relevant portions of which are incorporated herein by reference. As diagramed in FIG. 2, at three days post transfection each of three transfections was split into two pools, one containing puromycin and methotrexate (MTX) at 10 μg/mL and 100 nM, respectively, and the other containing puromycin and MTX at 20 μg/mL and 200 nM, respectively. Once the pools had recovered at least 85% viability as measured by trypan blue staining, a second phase of selection was initiated. In this second phase, each phase 1 pool was split into two pools, one of which contained puromycin and MTX at 30 μg/mL and 500 nM, respectively, and the other of which contained puromycin and MTX at 50 μg/mL and 1 μM, respectively. Once the phase 2 selection pools reached 90% viability as measured by trypan blue staining, they were evaluated for anti-hPD1 antibody titer in fed batch productions cultured for 10 days. Pool 3-2-5 was selected based on antibody titer. These cells were frozen in vials in CD FortiCHO™ medium containing DMSO as described above to make an RCB.

To obtain a clonal cell line, a vial of the frozen cells from the RCB described immediately above was thawed into CD FortiCHO™ medium containing 8 mM glutamine, 1 μM MTX, and 1% anti-clumping agent (ThermoFisher, catalog number 0010057AE). After three days of growth, cells were centrifuged and resuspended in a chemically-defined medium supplemented with 8 mM glutamine, 1 μM MTX, and 1% anti-clumping agent. The cells were sub-cultured twice more, allowing three days of growth between each transfer.

Cells were then diluted to a final concentration of 300 cells/mL in semi-solid CloneMedia (Molecular Devices, San Jose, Calif.) supplemented with glutamine, 1 μM MTX, and 0.5% CloneDetect (Molecular Devices). See Molecular Devices, Application Note, Confident identification of monoclonal CHO-S cells grown in semi-solid media using the CloneSelect Imager available at https://www.moleculardevices.com/en/assets/app-note/reagents/confident-identification-of-monoclonal-cho-s-cells-grown-in-semi-solid-media-using-cloneselect#gref, which is incorporated herein by reference. Cells were seeded in 6-well plates at 2 mL per well and incubated at 36.5° C., 5% CO₂, and 90% relative humidity (RH). After 10 days of static culturing, a ClonePix™ 2 system (Molecular Devices) was used to screen the plates for fluorescence. The ClonePix™ 2 system transferred 356 colonies with high fluorescence into individual wells of 96-well plates containing 100 μL of chemically-defined medium. After four to five days of static culture, medium in each well was carefully aspirated and replaced with 50 μL of fresh medium. The medium was exchanged in this way every three to four days thereafter until the colonies reached 80% confluence. The medium was exchanged again, and the colonies were incubated for an additional three days.

The colonies were then evaluated for anti-hPD1 antibody expression and growth characteristics. The 356 expanded colonies described above were further expanded by transferring the colonies from the 96-well plates to spin tubes. These spin tube cultures were frozen in vials as described above to create RCBs to be used for single cell cloning. During the expansion process the cell lines were evaluated for expression and growth characteristics in fed-batch productions. The first production was done in 24-well microtiter plates. Based on antibody expression levels measured using a ForteBio Octet® system Protein A quantitation assay (Sartorius, Goettingen, Germany), 185 cell lines with high antibody expression and acceptable growth characteristics were expanded into spin tubes and a second fed-batch production was performed. Based on this evaluation, the G19G4 cell line was chosen for cloning by limiting dilution as described below, and an RCB of G19G4 was created as described above.

A vial of the G19G4 RCB was thawed in chemically defined medium and cultured. After several passages, cells were diluted into CD FortiCHO™ medium and plated for cloning by limiting dilution into 96-well plates. Wells were imaged three hours post plating (T=0) and on days 1, 3, 9, and 13. Clones from single cells (based on imaging) were expanded, screened for growth characteristics, and then evaluated for antibody production performance in fed-batch cultures. Clone G19G4-4B4 was selected as the lead clone based on its growth characteristics and antibody production performance and was expanded in a 1 L shake flask and frozen in chemically defined medium supplemented with DMSO to generate an RCB as described above.

To make a cell line expressing the anti-hCTLA4 antibody PSB105, two vectors, one encoding the HC and the other encoding the LC of PSB105, were made as follows. To construct a vector encoding the HC of the anti-hCTLA4 antibody PSB105, the sequence of a DNA fragment encoding SEQ ID NO: 13 (the amino acid sequence the HC of the anti-hCTLA4 antibody) was optimized for expression in hamster cells (C. griseus) using GeneOptimizer™ online software (GeneArt, ThermoFisher Scientific). The optimized DNA fragment, the sequence of which is provided in SEQ ID NO: 14, was chemically synthesized. This DNA fragment encodes an HC that has the following amino acid alterations at the following positions (as defined by Edelman et al., supra): K147D, F170C, V173C, C220G, R255K, D399R, and K409E (corresponding to positions 148, 171, 174, 221, 256, 400, and 410 in SEQ ID NO: 13). Some of these alterations (K147D, F170C, V173C, C220G, along with anti-hCTLA4 LC alterations S131K, Q160C, S162C, and C214S) ensure cognate HC/LC pairing. Others (D399R, and K409E) ensure formation of homodimeric HC/HC pairs. One (R255K) causes an increased in vivo clearance rate and/or a decreased in vivo half life (t_{1/2}). This synthesized DNA fragment was amplified by PCR, and the resulting fragment was purified on an agarose gel as described above. The purified fragment was digested with SapI and ligated into a M268-c vector (Atum, Newark, Calif.) that had been digested with SapI. The ligated mixture was introduced into E. coli cells, colonies were picked, and the plasmid inserts from these colonies were sequenced. A colony with a 100% match to the sequence encoding the HC of the anti-hCTLA4 antibody was streaked twice more, and the insert in the plasmid DNA from a colony from the second streak was sequenced. It matched the sequence encoding the HC of the anti-hCTLA4 antibody.

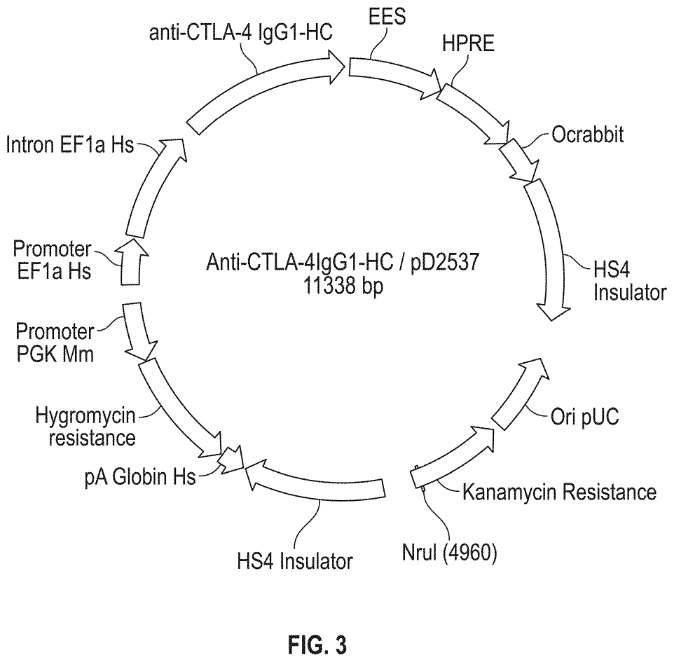

In a second step, the insert was transferred into a vector suitable for stable expression in mammalian cells, i.e., pD2537 (Atum), using an Electra kit (see https://www.atum.bio/catalog/regents/electra) according to the manufacturer's protocol. After a 15-minute incubation at room temperature, the Electra reaction was introduced into E. coli by electroporation. Cells were plated on Yeast Extract Glucose (YEG) agar plates containing containing 30 μg/mL kanamycin (thereby selecting for the pD2537 vector) and 10 mM p-chlorophenylalanine, which counterselects against the pM268-c plasmid. Colonies were screened for the presence of DNA encoding the anti-hCTLA4 HC, and the inserts in the plasmids in positive colonies were sequenced. A colony containing a plasmid matching the sequence of the DNA encoding of the HC of the anti-hCTLA4 antibody was then streaked twice more, and the sequence of both strands of the entire plasmid from a colony from the second streak was determined. It matched the vector sequence and the DNA sequence encoding the amino acid sequence of the anti-hCTLA4 HC. A map of the plasmid pD2537 containing the DNA encoding the HC of the anti-hCTLA4 HC is shown in FIG. 3.

A vector encoding the LC of the anti-hCTLA4 PSB105 antibody was constructed as follows. A DNA sequence encoding the LC of the anti-hCTLA4 antibody was optimized for expression in hamster (C. griseus) cells as described above, chemically synthesized, and amplified by PCR using primers including a SapI site. This PCR fragment was digested with SapI, ligated into SapI-digested pM268-c (Atum), and introduced into E. coli cells. DNA sequences of plasmid inserts from selected colonies were determined, and a colony containing a sequence encoding the LC of the anti-hCTLA4 antibody PSB105 was identified. This colony was expanded and used to make plasmid DNA for a second step.

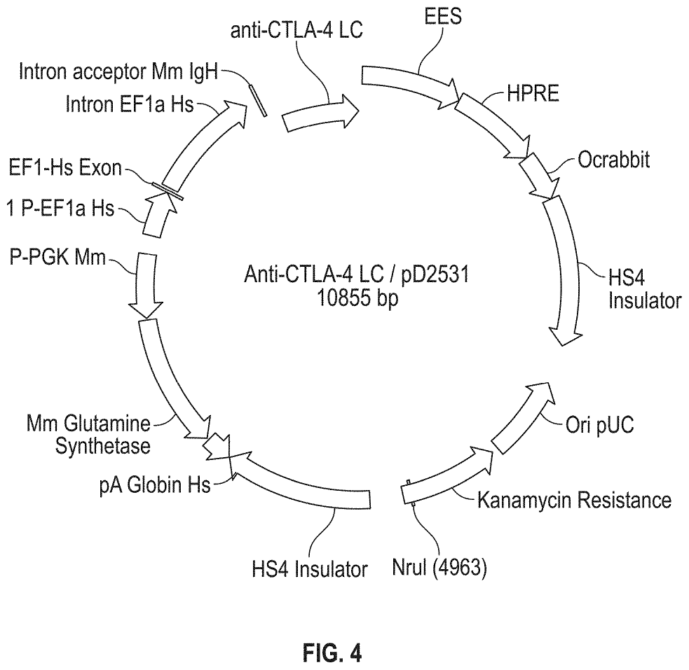

In the second step, an Electra reaction (to efficiently transfer the insert from one vector to another) was carried out using the pM268-c vector with inserted DNA encoding the LC of PSB105 (described above) and pD2531-EFM vector (Atum), which is a vector for stable expression in mammalian cells. The reaction was carried out for 15 minutes, and then introduced into E. coli cells, which were plated on 30 μg/mL kanamycin (thereby selecting for the pD2531-EFM vector) plus 10 mM p-cholorophenylalanine (to select against the pM268-c plasmid). Plasmid DNA from selected colonies was sequenced, and a colony containing an insert encoding the LC of PSB105 was identified. This colony was then streaked twice, and plasmid DNA from a colony from the second streak was sequenced. The sequence matched the vector sequence, and the inserted sequence encoded the LC of PSB105. Plasmid DNA was made from this colony. A map of this vector is shown in FIG. 4.

A CHO cell line expressing PSB105 was made by simultaneously introducing the mammalian expression plasmids encoding the HC and LC of PSB105 described above and diagrammed in FIGS. 3 and 4. In more detail, the two vectors were linearized with NruI-HF® (New England Biolabs, Ipswich, Mass.) and used to transfect CHO-S™ cells using FreeStyle™ MAX Reagent (Thermo Fisher) according to the manufacturer's instructions. See Freedom™ CHO-S™ Kit (catalog number A1369601) User Guide, Publication Number MAN0003505, Revision C.0, Thermo Fisher Scientific, which is incorporated herein by reference. Two days post transfection, selection was initiated by performing a complete media exchange into 40 mL CD-FortiCHO Medium supplemented with 25 μM methionine sulfoximine (MSX) and 200 μg/mL Hygromycin B (HGB). The transfection culture was split into three separate pools, each one seeded at either 3×10⁵, 5×10⁵, or 8×10⁵ cells/mL in T-150 flasks. Six days later, all pools were centrifuged, and the medium was carefully aspirated. The cell pellets were each resuspended in fresh medium containing 25 μM MSX and 200m/mL HGB at 3×10⁵ cells/mL and cultured in 125 mL vented shake-flasks. The pools were subsequently passaged as described immediately above until the viabilities were all >90% (as measured by trypan blue staining), at which point they were assessed for antibody expression in an 11-day fed-batch production. Based on expression level, the pool that was generated by initial seeding at 3×10⁵ cells/mL in the T-150 flask, which was called PSB105 Pool 2.03, was selected for producing PSB105 in a 5 L stirred-tank bioreactor.

Finally, PSB103 and PSB105 antibodies were made in parallel by separately culturing, respectively, the G19G4-4B4 cell line and PSB105 Pool 2.03, recovering antibody from the cell supernatants of these cultures, and purifying the antibody on a Protein A column using a single step elution, rather than a gradient elution.

### Example 2: Assessing the Binding Specificity of Anti-hPD1 and Anti-hCTLA4 Antibodies

The specificities of binding of the anti-hPD1 and anti-hCTLA4 antibodies, i.e., PSB103 and PSB105, were assessed by a solid phase Enzyme Linked Immuno-Sorbent Assay (ELISA) measuring binding to hPD1, hCTLA4, and other members of the CD28 family.

Briefly, 96-well, flat bottom microtiter plate wells were coated with 1 μg/mL of a capture molecule, which was either (1) the extracellular domain of human PD1 fused to an Fc fragment (hPD1.Fc), (2) the extracellular domain of human CTLA4 fused to an Fc fragment (hCTLA4.Fc), (3) the extracellular region of human PDL1 fused to a histidine-avi tag (which enables the efficient purification (histidine tag) of the protein and labeling of the protein (avi tag) with biotin) (hPDL1-his-avi), (4) the extracellular domain of murine PD1 fused to a histidine-avi tag (mPD1-his-avi), or (5) the extracellular domain of human CD28 fused to an Fc fragment (hCD28.Fc; R & D Systems catalog number 342-CD). Plates were sealed with an adhesive strip and incubated overnight at 18-24° C. Plates were washed in 1× phosphate buffered saline (PBS) with 0.05% Tween-20. Plates were blocked by adding 300 μL of Block Buffer (1×Dulbecco's phosphate buffered saline (DPBS) with 1% bovine serum albumin (BSA)) to each well and incubating one hour at room temperature. Plates were washed as described above.

A primary antibody (either PSB103 or PSB105) was added to each well in a volume of 100 μL. Multiple wells containing the same primary antibody in a serial dilution series were tested. Plates were sealed with an adhesive strip, incubated for one hour at room temperature, and washed as described above. Then 100 μL of polyclonal goat anti-human kappa light chain conjugated to horse radish peroxidase (HRP) (Sigma catalog number A7164) diluted 1:10000 in reagent diluent (Dulbecco's phosphate buffered saline (DPBS) with 0.05% Tween-20 and 0.1% BSA) was added to each well. The plates were again sealed with adhesive strips, incubated for one hour at room temperature, and washed as above. Then 100 μL of TMB (3,3′,5,5′-tetramethylbenzidine) Substrate Solution (from Pierce™ TMB Substrate Kit, ThermoFisher catalog number 34021) was added to each well, and the plates were incubated for 20 minutes at room temperature, avoiding placing the plates in direct light. Finally, 50 μL of Stop Solution from the Pierce™ TMB Substrate Kit was added to each well, and the optical density of each well was determined with a microplate reader set to 450 nm.

Results are shown in FIG. 5, panels A and B. PSB103 showed binding to hPD1, but not to mPD1, hPDL1, hCD28, or hCTLA4. Similarly, PSB105 showed binding to hCTLA4, but not to hPD1, mPD1, hPDL1, or hCD28. Thus, both PSB103 and PSB105 exhibited specific binding to their antigens.

### Example 3: Single Dose Pharmacokinetics of PSB103 and PSB105 in Cynomolgus Monkeys

Single dose pharmacokinetic properties of PSB103 in cynomolgus monkeys were assessed as follows. Two male and two female, protein naïve cynomolgus monkeys (i.e., monkeys who had not been previously dosed with a human antibody) of Cambodian origin (4.129 to 5.971 kg and 5 to 7 years of age) were placed into one of two study groups (n=2, one male and one female/group) and were acclimated to the study room for seven days prior to the start of dosing. Monkeys in one group received PSB103 and in the other group received an unrelated IgG4 antibody. PSB103 and the IgG4 antibody were injected at a dose of 5 mg/kg on Day 1 of the study by one slow intravenous (IV) bolus injection. The day prior to administration is denoted as Day −1, and days prior to that were numbered sequentially as Day −2, Day −3, etc. Days following Day 1 were numbered sequentially thereafter as Day 2, Day 3, etc. Body weights were recorded on Days −4, −1, 9 and 23, and clinical observations were performed on select days during acclimation and twice on each day in-life. Blood samples, approximately 0.5 mL, were collected pre-dose and at 0.083 (5 minutes), 0.5 (30 minutes), 2, 8, 16, 24, 72, 144, 240, 336, 504, and 672 hours after dose administration. Serum was obtained by centrifugation at 2,000×g at room temperature for 15 minutes. Each sample was divided into two aliquots, i.e., aliquots 1 and 2.

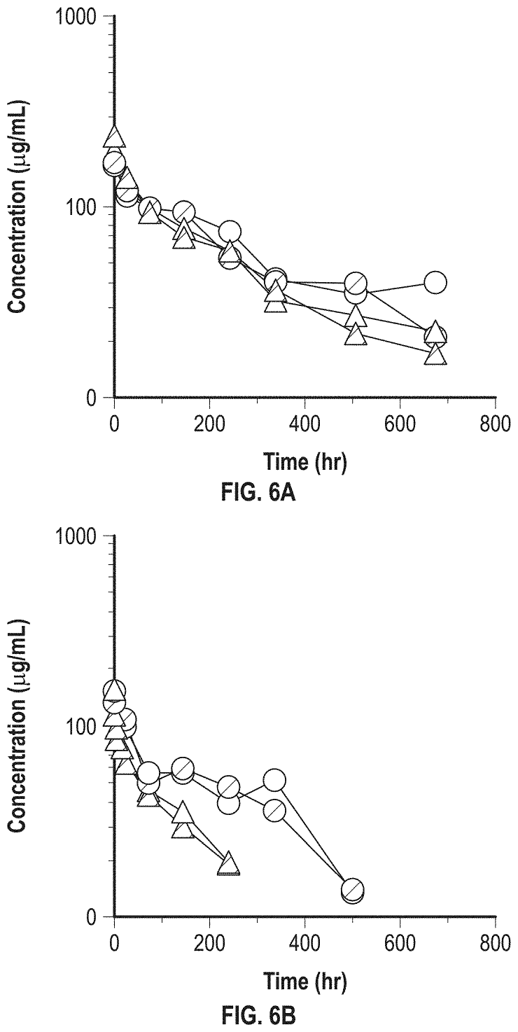

Sample bioanalysis was performed by a validated ELISA protocol using an anti-idiotypic antibody against PSB103. Briefly, an anti-idiotypic antibody against PSB103 (3G12) was used to pre-coat the microplate wells. After blocking and washing, samples (including test samples, blanks, standards, and quality control samples) were added to the wells and the plates were incubated and then washed. Then a biotinylated version of the anti-idiotypic 3G12 (bio-3G12) antibody was added to the wells. The bound bio-3G12 was detected with horseradish peroxidase (HRP) labeled streptavidin. The development solution containing tetramethylbenzidine (TMB), a substrate for HRP, was added to the microplate wells and resulted in a colorimetric signal at 450 nm proportional to the concentration PSB103 in the samples. The conversion of the optical density data to concentrations of PSB103 in the samples was done by comparison to a concurrently analyzed calibration curve regressed according to a four-parameter logistic model. The lower limit of quantitation (LLOQ) of PSB103 was 50 ng/mL. FIG. 6 shows a semi-log plot of these data.

The pharmacokinetic (PK) evaluation was performed using the individual serum concentrations and nominal time data with the noncompartmental analysis model plasma (200-202) IV bolus in validated Phoenix WinNonlin®, version 6.1 software (Pharsight Corporation). The nominal PK blood collection time points were pre-dose and at 0.083 (5 minutes), 0.5 (30 minutes), 2, 8, 16, 24, 72, 144, 240, 336, 504, and 672 hours post-dose. The area-under-the-serum-concentration-time-curve (AUC) was estimated by the linear log trapezoidal rule, and time points for estimating lambda z (λz) were selected by the software with best fit and uniformly weighted concentration data for the regression. These data are shown in Table 1 below.

TABLE 1
Pharmacokinetic parameters of PSB103 and an IgG4 isotype control
antibody following a single dose in cynomolgus monkeys

| Test | | T_{1/2} | Cₘₐₓ | AUC₀₋ₗₐₛₜ | AUC₀₋∞ | V_{z} | Cl |
|---|---|---|---|---|---|---|---|
| antibody | Parameter | (hr) | (μg/mL) | (hr* μg/mL) | (hr* μg/mL) | (mL/kg) | (mL/hr/kg) |
| PSB103 | n^{a} | 4 | 4 | 4 | 4 | 4 | 4 |
| | mean | 297 | 204 | 37300 | 48800 | 43.9 | 0.106 |
| | SD^{b} | 64.8 | 23.5 | 3540 | 10800 | 1.05 | 0.0203 |
| IgG4 | n^{a} | 4 | 4 | 4 | 4 | 4 | 4 |
| antibody | mean | 181 | 138 | 16900 | 21000 | 64.5 | 0.270 |
| | SD^{b} | 51.0 | 19.9 | 8060 | 8420 | 9.27 | 0.108 |

^{a}This includes the sample taken from each of the two monkeys in the group at each time point, each of which was divided into two separate aliquots for analysis.
^{b}SD means standard deviation.

Differences in PK parameters between aliquots for an individual animal or between animals were attributed to the variability in the bioanalytical data and limited numbers of animals. As expected for an IV bolus dose administration, the highest serum concentration (Cₘₐₓ) was generally at the first time point, 0.083 hours (Tₘₐₓ).

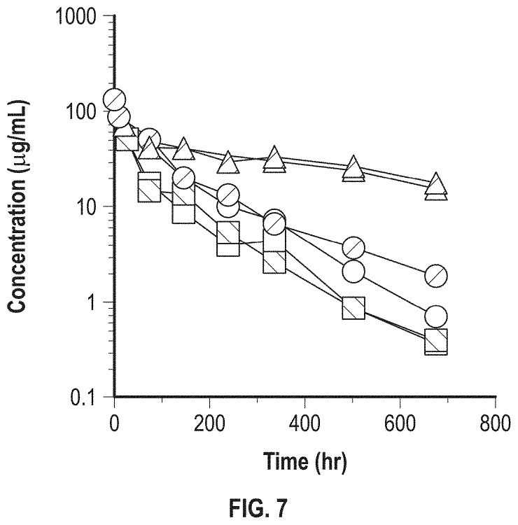

A second single-dose PK study assessed PK parameters of anti-hCTLA4 antibody PSB105. Six protein-naïve cynomolgus monkeys were separated into three groups, each containing one male and one female monkey. The three groups received a single dose of PSB105 (group 1), an unrelated human IgG1 antibody (group 2), or a commercially available anti-hCTLA4 antibody called ipilimumab (group 3) via bolus IV injection at a dose of 3 mg/kg. The nominal blood collection time points were pre-dose and at 0.083 (5 minutes), 0.5 (30 minutes), 2, 8, 16, 24, 72, 144, 240, 336, 504, and 672 hours post-dose. Serum was prepared as described above, and serum concentrations of the test antibodies were measured by a validated ELISA method using an anti-idiotypic antibody against PSB105 (3G4). This assay was performed as described above for PSB103 except that 3G4 and a biotinylated version of it (bio-3G4) were used instead of 3G12 and bio-3G12. The LLOQ of PSB105 was 250 ng/mL. FIG. 7 shows these data.

The PK parameters were determined as described above and are shown in Table 2 below.

TABLE 2
Pharmacokinetic parameters of PSB105, an unrelated IgG1 antibody,
and ipilimumab following a single dose in cynomolgus monkeys.

| Test | | T_{1/2} | Cₘₐₓ | AUC₀₋ₗₐₛₜ | AUC₀₋∞ | V_{z} | Cl |
|---|---|---|---|---|---|---|---|
| antibody | Parameter | (hr) | (μg/mL) | (hr* μg/mL) | (hr* μg/mL) | (mL/kg) | (mL/hr/kg) |
| PSB105 | n^{a} | 2 | 2 | 2 | 2 | 2 | 2 |
| | mean | 109 | 116 | 6080 | 6140 | 76.6 | 0.489 |
| | SD^{b} | 1.66 | 26.1 | 283 | 287 | 4.75 | 0.0229 |
| IgG1 | n^{a} | 2 | 2 | 2 | 2 | 2 | 2 |
| antibody | mean | 125 | 153 | 10100 | 10300 | 52.3 | 0.291 |
| | SD^{b} | 21.1 | 4.67 | 440 | 631 | 5.69 | 0.0177 |
| ipilimumab | n^{a} | 2 | 2 | 2 | 2 | 2 | 2 |
| | mean | 397 | 128 | 22500 | 31900 | 53.5 | 0.0948 |
| | SD^{b} | 104 | 4.67 | 93.1 | 3740 | 7.80 | 0.0111 |

^{a}This includes the sample taken from each of the two monkeys in each group at each time point.
^{b}SD means standard deviation.

Taken together with the data in Table 1, these data indicate that PSB103 has a t_{1/2} in cynomolgus monkeys (297 hours) that is almost three times as long as that of PSB105 (109 hours), indicating that PSB103 would likely persist in the blood stream longer than PSB105. Moreover, PSB105 also has a t_{1/2} in cynomolgus monkeys that is much shorter than that of ipilimumab (397 hours), which is an approved anti-hCTLA4 antibody.

### Example 4: Creation of a Mammalian Host Cell Line Expressing Both PSB103 and PSB105

The following describes the creation of a CHO host cell line expressing PSB103 and PSB105. As a first step, an RCB of CHO-S™ cells (ThermoFisher Scientific) was made as described in Example 1.

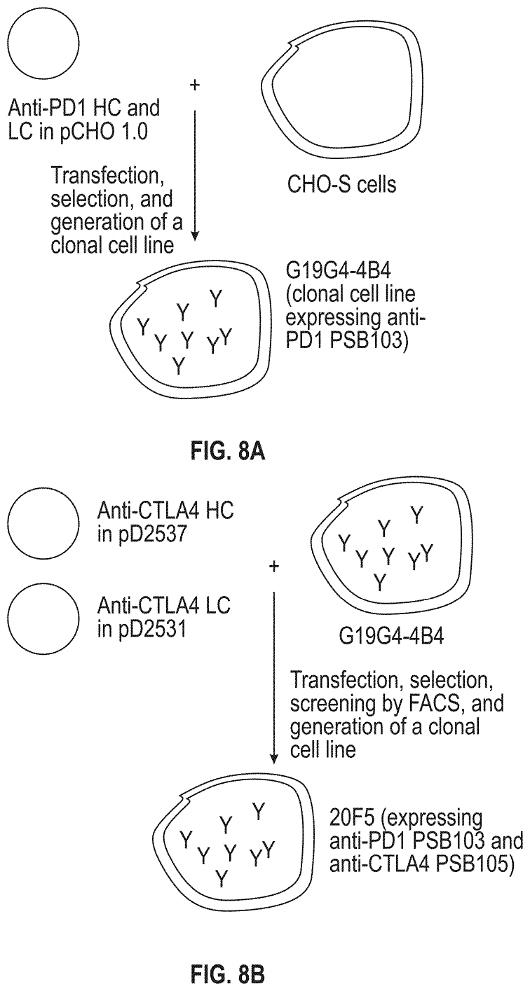

Thereafter, as diagrammed in FIG. 8, a cell line expressing both anti-hPD1 antibody PSB103 and anti-hCTLA4 antibody PSB105 was created in two steps. First, one vial from the clonal cell line G19G4-4B4 RCB (which expresses PSB103) described above in Example 1 was thawed and expanded in a 125 mL flask containing 19 mL CD FortiCHO™ medium supplemented with 8 mM glutamine and 1 μM MTX. Cells were passaged by dilution every two to three days for two weeks and then transfected. Prior to transfection, the plasmids encoding the HC and LC of anti-hCTLA4 antibody PSB105 described above and diagrammed in FIGS. 3 and 4 were linearized with NruI-HF® (New England Biolabs, Ipswich, Mass.). Two transfections, each using 3×10⁷ cells plus 25 μg each of the plasmids encoding the HC and LC of anti-hCTLA4 antibody PSB105 described above, were performed using FreeStyle™ MAX Reagent (ThermoFisher Scientific) using the manufacturer's instructions. Transfected cells were seeded into two flasks containing 30 mL of CD FortiCHO™ medium supplemented with 8 mM glutamine and 1 μM MTX with a cell concentration of 10⁶ cells/mL. Flasks were incubated for 48 hours at 37° C., 5% CO₂ on a 25 mm orbital diameter shaker platform rotating at 150 RPM.

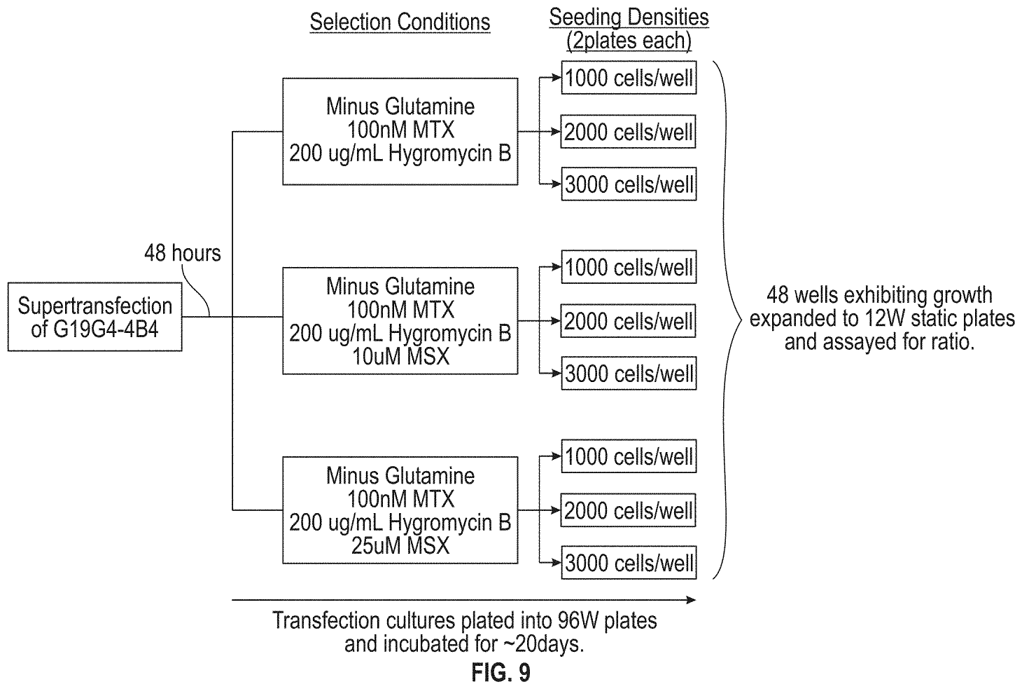

Thereafter, selection was initiated by combining the two flasks, separating the resulting culture into three pools, and seeding the cells into 96-well plates using about 1000-3000 cells/well in 0.5× glutamine synthetase expression (GS) medium supplement (Sigma), 100 nM MTX, 200 μg/mL hygromycin B (HGB), and either no methionine sulfoximine (MSX), 10 μM MSX, or 25 μM MSX. This selection scheme is diagrammed in FIG. 9. The MTX selects for the presence of the vector encoding anti-hPD1 antibody PSB103. The HGB selects for the presence of vector encoding the HC of anti-hCTLA4 antibody PSB105. The absence of glutamine and the presence of the glutamine synthetase inhibitor MSX select for the presence of the vector encoding the LC of anti-hCTLA4 antibody PSB105, which included DNA encoding M. musculus glutamine synthetase. Plates were incubated for 12 days and then imaged to identify wells showing growth. Forty-eight wells were selected for expansion into successively larger wells until they were seeded into 12-well plates.

When the cells reached confluence in the 12-well plates, the supernatants were analyzed by ELISA to identify wells expressing anti-hCTLA4 antibody PSB105 and anti-hPD1 antibody PSB103 at an approximate 1:2 ratio. Selected wells from each of the three pools (no MSX, 10 μM MSX, or 25 μM MSX) were analyzed to determine what percentage of the total antibody produced by the cells was anti-hPD1 antibody PSB103. Data is shown in Table 3 below.

TABLE 3
Percentage anti-hPD1 antibody PSB103 in wells
containing in varying concentrations of MSX

| Selection condition | Well | % anti-hPD1 PSB103 |
|---|---|---|
| 200 μg/mL HGB, 100 nM MTX | 5C6 | 75 |
| | 5C9 | 59 |
| | 5D2 | 74 |
| | 5D6 | 46 |
| | 5D8 | 68 |
| | 5E6 | 68 |
| | 5E7 | 84 |
| | 5G7 | 19 |
| | 6B3 | 79 |
| | 6B7 | 47 |
| | 6C6 | 58 |
| | 6D6 | 69 |
| | 6D7 | 73 |
| | 6E6 | 59 |
| | 6F6 | 56 |
| | 6G5 | 68 |
| Average | | 62 |
| 200 μg/mL HGB, 100 nM MTX, | 10C8 | 10 |
| 10 μM MSX | 10D6 | 22 |
| | 11B4 | 49 |
| | 11C9 | 40 |
| | 11D5 | 57 |
| | 11D7 | 48 |
| | 11E5 | 38 |
| | 11E7 | 33 |
| | 11E10 | 39 |
| | 11G7 | 31 |
| | 12B9 | 60 |
| | 12C4 | 32 |
| | 12C8 | 29 |
| | 12D10 | 58 |
| | 12E4 | 54 |
| | 12E8 | 31 |
| | 12F7 | 63 |
| Average | | 41 |
| 200 μg/mL HGB, 100 nM MTX, | 17B7 | 5 |
| 25 μM MSX | 17C4 | 1 |
| | 17C7 | 29 |
| | 17D8 | 28 |
| | 17E6 | 33 |
| | 17F6 | 13 |
| | 18C11 | 0 |
| | 18D4 | 44 |
| | 18D6 | 44 |
| | 18D7 | 42 |
| | 18E3 | 22 |
| | 18E5 | 25 |
| | 18E11 | 30 |
| | 18F5 | 19 |
| | 18F7 | 24 |

| | |
|---|---|
| Average | 24 |

The data in Table 3 indicates that the wells without MSX produced a higher proportion of anti-hPD1 antibody PSB103 on average. Fifteen of the 16 wells without MSX tested, i.e., all except 5G7, were pooled together in a 50 mL conical tube, which was incubated a further four days at 37° C., 5% CO₂ on a 25 mm orbital diameter shaker platform rotating at 225 RPM. Cells were diluted to 2.5 cells/mL in CD FortiCHO™ medium supplemented with 6 mM glutamine and seeded into 96-well plates at 0.5 cells/mL. Wells were imaged two hours after plating and on days 2, 13, and 21 to identify wells that contained only one cell. One hundred and forty single cell clones were expanded.

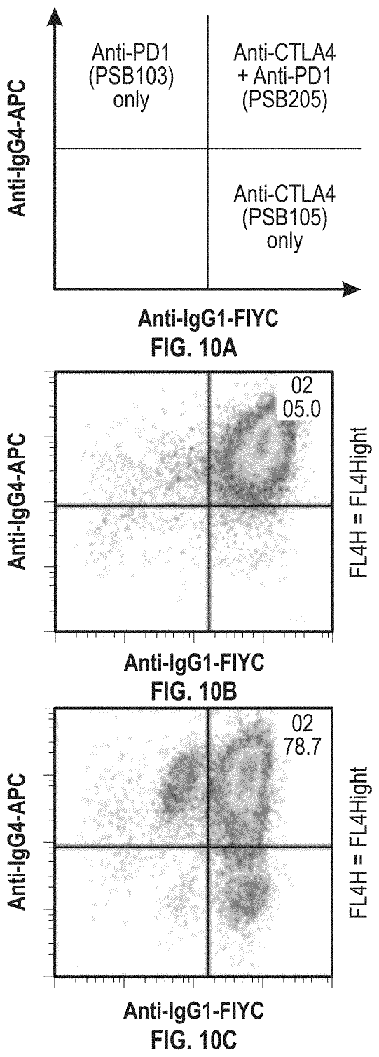

One hundred and twenty-five of these cell lines were screened for expression of anti-hPD1 antibody PSB103 (an IgG4 antibody) and anti-hCTLA4 antibody PSB105 (an IgG1 antibody) by first permeabilizing the cells and then staining for intracellular expression of IgG4 and IgG1. Clonal cell lines in which some cells expressed predominantly only IgG4 or only anti-IgG1 antibodies (as illustrated in FIG. 10, panel C) were not selected for further analysis. Clonal cell lines in which almost all cells expressed both anti-hPD1 and anti-hCTLA4 antibodies (as illustrated in FIG. 10, panel B) were selected for further analysis.

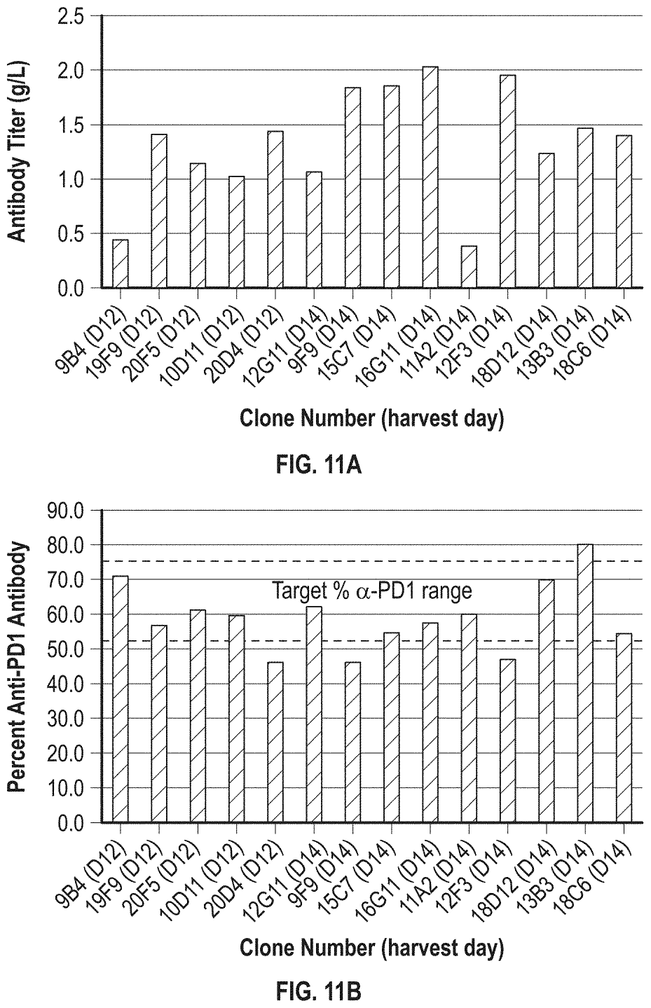

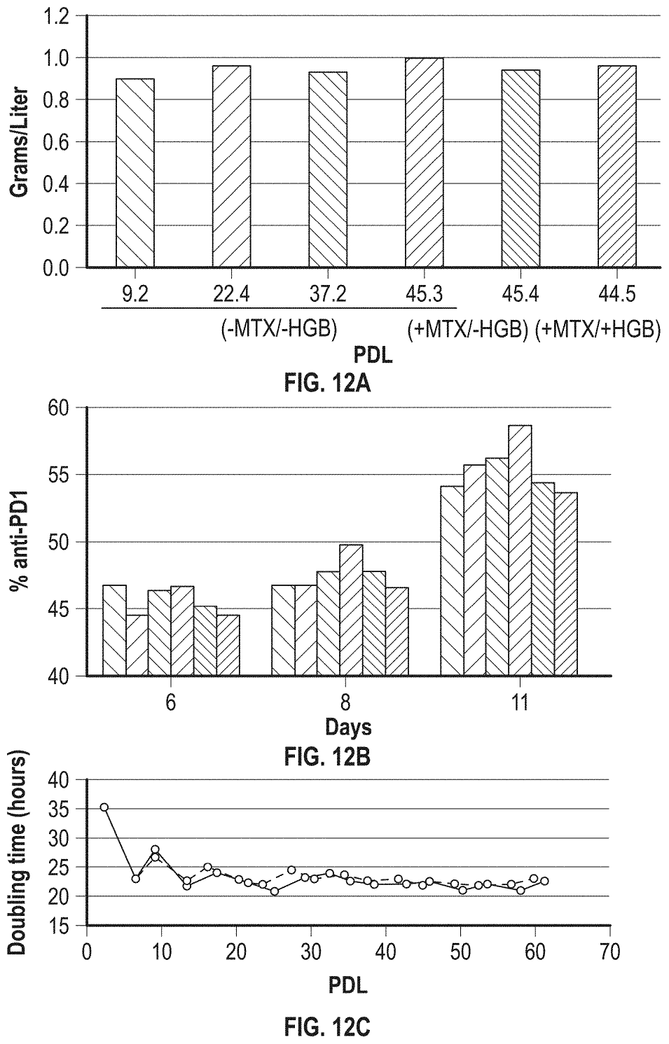

Fourteen of these cell lines were expanded in shake flasks, and antibodies were recovered from the cell supernatant 12 and/or 14 days after the start of the culture using a Protein A column. The amount of antibody was assessed by measuring absorbance at 220 nm and comparing results with absorbance of a dilution series of a standard protein at known concentrations. The percent of the total antibody that was anti-hPD1 antibody (% anti-hPD1) was determined by pH gradient cation exchange chromatography (CEX) as described by Zhang et al. (2013), Improving pH gradient cation-exchange chromatography of monoclonal antibodies by controlling ionic strength, J. Chromatography A 1272: 56-64, which is incorporated herein by reference. These data are shown in Table 4 below and FIG. 11.

TABLE 4
Percent anti-hPD1 in clonal cell lines*

| Clonal cell | | |
|---|---|---|
| line | Day harvested | Percent anti-hPD1 |
| 9F4 | 12 | 71.1 |
| 19F9 | 12 | 56.8 |
| 20F5* | 12 | 61.2 |
| 10D11* | 12 | 59.8 |
| 20D4 | 12 | 46.1 |
| 12G11 | 14 | 62.3 |
| 9F9 | 14 | 46.4 |
| 15C7* | 14 | 54.8 |
| 16G11* | 14 | 57.7 |
| 11A2 | 14 | 60.1 |
| 12F3 | 14 | 47.1 |
| 18D12 | 14 | 70.0 |
| 13B3* | 14 | 80.1 |
| 18C6 | 14 | 54.5 |

*lines selected for further analysis

As indicated in Table 4 above, five clonal cell lines producing from about 50-75% anti-hPD1 antibody and at least 1 g/L total antibody were selected for further analysis. These cells were grown in bench scale bioreactors, and cell doubling time was determined. Antibodies were recovered from the cell supernatants, and antibody titer and percent anti-hPD1 antibody were determined as described above. The purity of the antibodies in the antibody mixture was assessed by performing size exclusion chromatography (SEC). These data appear in Table 5 below.

TABLE 5
Characterization of antibody expression of selected clonal cell lines

| | Total | | | | Percent of |
|---|---|---|---|---|---|
| Cell | antibody | Days in | Percent | Doubling | antibody in |
| line | titer (g/L) | culture | anti-hPD1 | time (hours) | SEC main peak |
| 16G11 | 2.02 | 14 | 57.7 | 30.8 | 99.5 |
| 15C7 | 1.85 | 14 | 54.8 | 27.9 | 99.5 |
| 10D11 | 1.03 | 12 | 59.8 | 23.4 | 99.5 |
| 20F5 | 1.14 | 12 | 61.2 | 25.0 | 99.3 |
| 13B3 | 1.48 | 14 | 80.1 | 23.7 | 98.7 |

Cell line 20F5 was selected for further characterization. An RCB of 20F5 cells was created as described above. Further experiments were done to determine whether the population doubling level (PDL), i.e., the number of times the cells in a population have doubled since establishment of the RCB, and/or the presence of methotrexate (MTX) or hygromycin B (HGB) in the medium affected overall antibody expression, the anti-hCTLA4:anti-hPD1 antibody ratio, and/or the cell doubling time.

Briefly, a vial of the 20F5 RCB was thawed into medium containing 100 nM MTX and 200 μg/mL HGB (+MTX/+HGB medium). Three days post-thaw this culture was used to create an additional culture in medium containing 100 nM MTX and lacking HGB (+MTX/−HGB medium). The resulting two cultures were aged for about 60 PDLs by passaging every 3-4 days in their respective media. Doubling time was monitored, producing the data shown in FIG. 12, panel C. These data indicated that the doubling time of 20F5 was fairly constant at PDLs from about 10 to 60 and was similar in +MTX/+HGB and +MTX/−HGB media.

During the time the cultures in +MTX/+HGB and +MTX/−HGB media mentioned above were being aged, vials of each culture were frozen (as described above for making an RCB) at PDLs of about 13 and about 28. At a PDL of about 35, the culture in +MTX/−HGB medium was used to create another culture in medium lacking both MTX and HGB (−MTX/−HGB medium), which was cultured until it reached a PDL of 45.3. At this time, it was used to inoculate a fed batch culture in −MTX/−HGB medium, which produced data shown in the fourth bar from the left in FIG. 12, panel A. Further, when the culture in +MTX/−HGB medium reached a PDL of 45.4, it was used to inoculate a fed batch culture in −MTX/−HGB medium, which produced the data shown in the fifth bar from the left in FIG. 12, panel A.

In addition, when the culture in +MTX/+HGB medium reached a PDL of 44.5, it was used to initiate a fed batch culture in −MTX/−HGB medium, which was used to produce the data shown in the rightmost bar in FIG. 12, panel A.

Additionally, vials of cells from the +MTX/−HGB culture frozen at PDLs of about 13 and 28 were thawed into −MTX/−HGB medium and cultured in −MTX/−HGB medium until they reached PDLs of 22.4 and 37.2, respectively. At this time, these cultures were used to inoculate fed batch cultures in −MTX/−HGB medium, which produced data shown in the second and third bars from the left, respectively, in FIG. 12, panel A.

Finally, a vial of cells from the 20F5 RCB was thawed −MTX/−HGB medium and cultured in −HGB/−MTX medium until it reached a PDL of 9.2, when the culture was used to inoculate a fed batch culture in −MTX/−HGB medium that produced the data shown in the leftmost bar in FIG. 12, panel A.

Cell culture supernatant samples were taken from all fed batch cultures described above at 6, 8, and 11 days after the initiation of each culture to determine the total amount of antibody produced and the percent of antibody that was anti-hPD1 antibody. Antibody in the day 11 samples was recovered using a Protein A column. The amount of antibody recovered was assessed by measuring absorbance at 220 nm and comparing results with absorbance of a dilution series of an antibody at known concentrations. These results are shown in FIG. 12, panel A.

The percent of anti-hPD1 antibody was measured directly from the cell culture supernatant samples collected on days 6, 8, and 11 using an in-house method developed using an Octet Red system (Sartorious) equipped with streptavidin (SA) sensors (Sartorius, catalog #: 18-5019). Briefly, anti-idiotypic antibodies (anti-id Ab) specific for the anti-hPD1 antibody and anti-hCTLA4 antibody were biotinylated at a 1:1 biotin/anti-id Ab molar ratio using a commercially available kit following the manufacturer's protocol (Thermo Scientific, catalog #: 21955). The biotinylated anti-idiotypic antibodies were then immobilized to SA sensors to generate two sets of sensors, one set using the anti-hPD1 anti-id Ab and the other set using the anti-hCTLA4 anti-id Ab. Purified anti-hPD1 and anti-hCTLA4 were serially diluted in PBS to generate standard curves of each antibody at known concentrations and added to an assay plate which also contained diluted day 6, 8, and 11 cell supernatant samples. The SA sensors with immobilized anti-hPD1 anti-id were used to measure the amount of anti-hPD1 antibody in the cell culture samples, and the SA sensors with immobilized anti-CTLA4 anti-id were used to measure the amount of anti-CTLA4 antibody in the cell culture samples. Results were compared to the results from the purified samples of each antibody at known concentrations to determine the amounts of antibody present in the cell culture samples. The total amount of antibody present in each sample was determined by adding the amount of anti-hPD1 and anti-CTLA4 detected in the sample. The percent of anti-hPD1 antibody present in each sample was determined by dividing the amount of anti-hPD1 measured in each sample by the total amount of antibody present in each sample. These data are shown in FIG. 12, panel B.

Taken together, the data in FIG. 12 indicated that PDL did not substantially affect anti-hCTLA4:anti-hPD1 antibody ratios or overall antibody expression in cell populations with PDLs within the tested ranges. FIG. 12, panels A and B. Further, overall antibody titer and the anti-hCTLA4/anti-hPD1 antibody ratio was essentially the same in all cultures, indicating that the various media tested had no effect on these indices. FIG. 12, panels A and B. In addition, the doubling time of cell line 20F5 was fairly constant in a culture at PDLs from about 10 to 60, regardless of whether the medium was +MTX/−HGB/or +HGB/+MTX medium. FIG. 12, panel C. The mixture of PSB103 and PSB105 produced by the 20F5 cell line is referred to herein as PSB205.

### Example 5: Making PSB205

PSB205 was made as follows. The 20F5 cell line was cultured, and the cell supernatant was harvested. PSB205 was purified from the cell supernatant using Protein A affinity chromatography, where the antibody mixture was eluted from the Protein A in a single step, rather than with a gradient. Other steps can optionally be added to increase the purity of the preparation such as, e.g., various column chromatography steps such as anion and/or cation exchange chromatography, reverse phase chromatography, hydrophobic interaction chromatography, and/or size exclusion chromatography, plus various precipitation strategies, dialysis, and/or any of a variety of filtration steps.

Finally, the ratio of PSB105 to PSB103 as a weight/weight (w/w/) percentage was determined for two different lots of PSB205, one used for toxicology studies (PSB205-Tox) and one produced using Good Manufacturing Practice (GMP) protocol (PSB205-GMP). The relative concentrations of the antibodies were determined using hydrophobic interaction high-performance liquid chromatography (HI-HPLC) using a decreasing salt concentration to separate the antibodies. Antibodies were detected with UV light. The distinct peak areas of PSB103 and PSB105 were integrated and summed in parallel. The ratio of each antibody was determined by dividing the area of each of the two the antibody peaks by the sum of the areas of both antibody peaks. These data are tabulated in Table 6 below.

TABLE 6
W/w percentages of PSB103 and PSB105 in lots of PSB205

| Lot | PSB103 (% w/w) | PSB105 (% w/w) |
|---|---|---|
| PSB205-Tox | 67.3 | 32.7 |
| PSB205-GMP | 69.3 | 30.7 |

These data indicate that ratio of PSB105:PSB103 in different lots is very comparable.

Non-reduced intact masses of the antibodies in PSB205-Tox and PSB205-GMP were measured by liquid chromatography-mass spectrometry (LC-MS). Similar data was obtained from species of PSB103 and PSB105 that had been isolated from a preparation of PSB205, which are referred to herein as PSB103-S and PSB105-S. More specifically, mass spectra were acquired by size-exclusion ultra-high performance liquid chromatography (SE-UPLC) coupled to a quadrupole time-of-flight (Q TOF) mass spectrometer with electrospray ionization (ESI). Results are shown in FIG. 13, panels A (PSB103-S), B (PSB105-S), and C (the PSB205 preparation from which PSB103-S and PSB105-S were isolated) and FIG. 14, panels A (PSB205-Tox) and B (PSB205-GMP). The sizes of the major antibody species detected corresponded to various glycosylated species of PSB103 and PSB105 (for a detailed explanation of N-glycosylated antibody species, see, e.g., Yang et al. (2016), Ultrafast and high-throughput N-glycan analysis for monoclonal antibodies, MAbs 8(4): 706-717, which is incorporated herein by reference), where, as explained below, the HCs of both antibodies lack the C-terminal lysine, and the N-terminal glutamine of the HC of both antibodies has been converted to pyroglutamic acid. The identities of the glycosylated species detected are explained in the Brief Description of FIG. 13. No major species of antibodies including the C-terminal lysine or an unmodified N-terminal glutamine were detected. The sizes derived from the results shown in FIG. 13 are shown in Table 7 below.

TABLE 7
Major glycoform masses of PSB205 and
its individual antibodies by LC-MS

| | | G0F/G0F | | G0F/G1F | |
|---|---|---|---|---|---|
| Samples | | PSB103 | PSB105 | PSB103 | PSB105 |
| Expected Mass | | 149312.8 | 147,607.6 | 149,475.0 | 147,769.7 |
| Detected | PSB205 | 149,319.7 | 147,609.8 | 149,478.0 | 147,773.0 |
| Mass | PSB103-S | 149,320.4 | N/A | 149,479.2 | N/A |
| | PSB105-S | N/A | 147,610.5 | N/A | 147,772.5 |

Differential scanning calorimetry (DSC) was used to determine the stability of PSB205. Theoretically, DSC measures the excess heat capacity of a protein in a solution versus a control solution without protein as a function of temperature change, during which structural unfolding transition is observed as an endothermic peak. The temperature at the midpoint of this transition is defined as the melting temperature (Tm). In this study, thermal stability was determined by DSC using a MicroCal VP-DSC capillary cell microcalorimeter. DSC and the analysis of its results is described in, e.g., Durowoju et al. (2017), Differential scanning calorietry—a method for assessing the thermal stability and conformation of protein antigen, J. Visualized Experiments 121: e55262, available at doi:10.3792/55262, which is incorporated herein by reference.

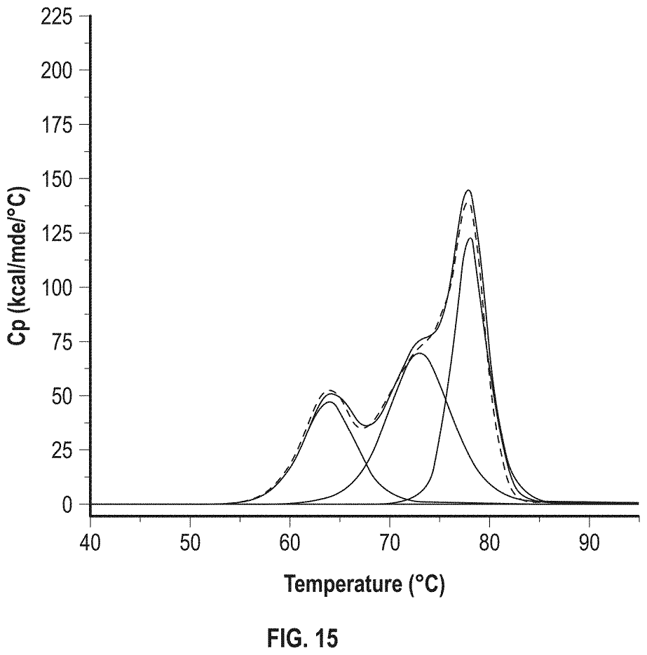

An exemplary resulting thermogram shown in FIG. 15 indicates that a GMP lot of PSB205 exhibited three thermal transitions. Both toxicology and GMP lots of PSB205 exhibited similar thermograms. A non-two-state model was fit to each thermal scan to obtain three Tm values (Tm1, Tm2, and Tm3). Specifically, the toxicology lot of PSB205 had Tm1, Tm2, and Tm3 values of, respectively, 64.6° C., 73.3° C., and 77.9° C. The GMP lot of PSB205 had Tm1, Tm2, and Tm3 values of, respectively, 64.0° C., 73.0° C., and 78.0° C. Thus, the observed thermal transitions varied little from lot to lot.

### Example 6: Structural Variants of PSB205

Due to the fact that PSB205 was produced in CHO cells, it was possible that structural variants, such as, e.g., post-translationally modified forms including glycosylated forms or forms comprising modified or deleted amino acids, would be present in preparations PSB205. As stated above, the HC and LC of the anti-hCTLA4 antibody in PSB205, i.e., PSB105, can be encoded by the nucleic acid sequences of SEQ ID NOs: 14 and 18. These sequences encode the amino acid sequences of SEQ ID NOs: 13 and 17, respectively. Similarly, the HC and LC of the anti-hPD1 antibody in PSB205, i.e., PSB103, can be encoded by the nucleic acid sequences of SEQ ID NOs: 2 and 6, which encode the amino acid sequences of SEQ ID NOs: 1 and 5. However, because of potential post-translational modifications, these sequences may not precisely define the structure of PSB105 and PSB103 when these antibodies are made in CHO cells transfected with polynucleotides comprising these nucleic acid sequences.

As explained above, non-reduced intact masses of the antibodies in PSB205 were measured by liquid chromatography-mass spectrometry (LC-MS). Specifically, mass spectra were acquired by size exclusion ultra performance liquid chromatography (SE-UPLC) coupled to a quadrupole time-of-flight mass spectrometer (Q TOF MS) with electrospray ionization (ESI). This was done for PSB205, as well as PSB105 and PSB103 isolated from a preparation of PSB205 by chromatography. These isolated preparations of PSB105 and PSB103 were called PSB105-S and PSB103-S, respectively. Raw data were analyzed with spectrum deconvolution software. As explained below, the N-terminal glutamine on the HCs of both PSB103 and PSB105 can be converted to pyroglutamic acid (see, e.g., Pyroglutamate in PubChem Compound Summary available at https://pubchem.ncbi.nlm.nih.gov/compound/Pyroglutamate) in most species present in PSB205, and the C-terminal lysine in both HCs can be deleted in most species present in PSB205. The sizes of the two most abundant antibody species in PSB205, i.e., 147,609.8 and 149, 319.7 daltons, are consistent with PSB105 and PSB103 species, respectively, having the two modifications mentioned immediately above plus GOF/GOF glycosylation (see, e.g., Yang et al., supra and the Brief description of FIG. 13) on each HC/HC pair, presumably attached to the well-known glycosylation site at N297 on each HC. This is a position numbered according to Edelman et al., supra, which corresponds to positions 296 and 298 in SEQ ID NOs: 1 and 13, respectively. The second most prominent two species detected at 147,769.7 and 149,475.0 daltons are consistent with PSB105 and PSB103 species having the two modifications mentioned immediately above plus GOF/G1F (ibid.) glycosoylation on the HC/HC pairs, again, presumably attached to N297. Thus, PSB205 contains specific glycosylated antibody species.

As mentioned above, the primary amino sequences of the antibodies in PSB205 were found to be modified in detected species of antibodies. This was ascertained by liquid chromatography tandem mass spectrometry (LC-MS/MS) tryptic peptide mapping. See, e.g., Jenkins et al. (2015), Recommendations for validation of LC-MS/MS bioanalytical methods for protein biotherapeutics, AAPS Journal 17(1): 17 pages (available at DOI: 10.1208/s12248-014-9685-5), which is incorporated herein by reference. LC-MS/MS was carried out using reverse phase ultrahigh performance liquid chromatography (RP-UPLC) with UV 215 nm detection coupled to a Q TOF MS with ESI.

Since PSB205 contains both PSB105 and PSB103, tryptic peptide maps of the individual purified antibody PSB105-S and PSB103-S were first compared to the tryptic peptide map of PSB205 to determine which tryptic peptides in the PSB205 map were from which antibody. Most of the expected individual peptides of PSB105 and PSB103 were detected in the tryptic peptide maps of PSB105-S and PSB103-S, respectively, and also in the peptide map of PSB205. Only a few short peptides of four or fewer amino acids were not detected, which was attributed to the limitations of the method.

However, some peptides did not correspond to their theoretically predicted sizes. Both PSB105 and PSB103 would be predicted to have an N-terminal glutamine residue in their HC based on the DNA sequences encoding these HCs. The N-terminal tryptic peptides of both PSB105 and PSB103 (in isolated form, as well as when part of a mixture in PSB205) had a size consistent with theoretical sizes of these peptides in the case where the N-terminal glutamine was converted to pyroglutamate. Similarly, the C-terminal amino acid of the HCs of both PSB105 and PSB103 would be predicted to be a lysine based on the DNA sequences encoding these HCs. However, the sizes of the C-terminal peptides of both PSB105 and PSB103 were consistent with theoretically determined sizes of these peptides without their C-terminal lysines. Thus, both PSB103 and PSB105 have a modification of their N-terminal glutamine and a deletion of their C-terminal lysine.

In addition, some deamidation of asparagine (N) residues was observed in certain tryptic peptides from both PSB103 and PSB105. This deamidation was detected by changes in sizes of tryptic peptides using the LC-MS/MS methods described above. A percentage was derived by dividing the peak area of a deamidated tryptic peptide by the sum of the peak areas of the deamidated peptide plus the same non-deamidated peptide. These results are summarized in Table 8 below.

TABLE 8
Percent deamidation of asparagine residues detected in certain
peptides from PSB103 and PSB105

| | | Percent deamidated | |
|---|---|---|---|
| | | peptide | |
| Deamidation | | PSB205- | PSB205- |
| site | Peptide sequence | Tox^{a} | GMP^{b} |
| PSB103 | SSQSLFNSGNQK (SEQ ID NO: 25) | 1.4% | 1.3% |
| LC: N31 | | | |
| PSB103 | FSGSGSGTDFTLTISSLQPEDVATYYC | 3.4% | 3.5% |
| LC: N96 | QNDHYYPYTFGGGTK (SEQ ID NO: 26) | | |
| PSB103 | SGTASVVCLLNNFYPR (SEQ ID NO: 27) | 1.8% | 1.9% |
| LC: N144 | | | |
| PSB103 | DYFPEPVTVSWNSGALTSGVHTFPAVLQSS | 1.5% | 1.6% |
| HC: N161 | GLYSL | | |
| | SSVVTVPSSSLGTK (SEQ ID NO: 28) | | |
| PSB103 | VVSVLTVLHQDWLNGK^{c} (SEQ ID NO: 29) | 18.4% | 18.5% |
| HC: N314 | | | |
| PSB103 | NQVSLTCLVK^{c} (SEQ ID NO: 30) | 1.3% | 1.2% |
| HC: N360 | | | |
| PSB103 | GFYPSDIAVEWESNGQPENNYK^{c} (SEQ ID | 23.1% | 21.1% |
| HC: N383 | NO: 31) | | |
| PSB103 | WQEGNVFSCSVMHEALHNHYTQK (SEQ ID | 3.2% | 3.2% |
| HC: N420 | NO: 32) | | |
| PSB105 | ASQSINSYLAWYQQKPGQAPRPLIYGVSSR | 38.4% | 36.3% |
| LC: N30 | (SEQ ID NO: 33) | | |
| PSB105 | VVCLLNNFYPR (SEQ ID NO: 34) | 3.1% | 3.1% |
| LC: N137 | | | |
| PSB105 | VVSVLTVLHQDWLNGK^{c} (SEQ ID NO: 29) | 18.4% | 18.5% |
| HC: N316 | | | |
| PSB105 | NQVSLTCLVK^{c} (SEQ ID NO: 30) | 1.3% | 1.2% |
| HC: N362 | | | |
| PSB105 | GFYPSDIAVEWESNGQPENNYK^{c} (SEQ ID | 23.1% | 21.1% |
| HC: N385 | NO: 31) | | |

^{a}This is the same toxicity lot for which mass data is shown in FIG. 14, panel A.
^{b}This is the same GMP lot for which mass data is shown in FIG. 14, panel B.
^{c}Sequence shared between PSB103 and PSB105.

These data show that the percent deamidation is essentially the same in the two lots of PSB205. The relatively high levels of deamidation at some aspargine residues could be explained by the tryptic digestion conditions, i.e., pH 8 at 37° C. overnight, which can induce deamidation.

### Example 7: Binding Kinetics of PSB103, PSB105, and PSB205 to their Antigens

The kinetics of the binding of PSB103 to the extracellular domains of human and cynomolgus monkey PD1 (hPD1 and cPD1) and PSB205 to the extracellular domain of hPD1 was determined using surface plasmon resonance (SPR) technology as measured by a Biacore 3000 optical biosensor equipped with a CM5 sensor chip according to the manufacturer's general protocol.

Initially the sensor chip was placed on the instrument and allowed to equilibrate overnight or longer. The running buffer (0.01 M HEPES pH 7.4, 0.15 M NaCl, 3 mM EDTA, 0.005% v/v Surfactant P20 (HBS-EP, GE Life Sciences (now Cytiva) catalog number BR100188)) was filtered and degassed prior to being placed on the system. Once the running buffer was connected, the Biacore 3000 was primed three times, and the “Normalize” system procedure was executed before running the experiment to calibrate the system's optics. All measurements occurred at 25° C.

Initially, about 8000 resonance units (RU) of a goat anti-human antibody capture antibody (Jackson Labs, catalog number 109-005-098) were immobilized on each of two flow cells of a CMS chip (GE Life Sciences catalog number BR100399) using an Amine Couple Kit (GE Life Sciences, BR100050). To assess binding of cynomolgus monkey PD1 to the PSB103 ligand, one flow cell of the CMS chip was used to capture about 100-200 RU of PSB103, and the other served as a reference chip for the experiment. The cPD1-his analyte (amino acids 1-167 of cPD1 with a histidine tag at the C-terminus; Sino Biological, catalog number 90311-C08H) was diluted in running buffer supplemented with 0.1% bovine serum albumin (BSA) to concentrations of 1.2, 3.7, 11.1, 33.3, 100, and 300 nM. These dilutions were injected into the test flow cell with captured PSB103 and the reference flow cell at a flow rate of 50 μL/min. The complex was allowed to associate and dissociate for 300 and 1500 seconds, respectively. The surfaces were regenerated with a 30 second injection of 10 mM glycine-HCl, pH 1.5. Duplicate injections of each analyte sample and a buffer blank were flowed over the reference and ligand-captured flow cells.

Scrubber 2 software version 2.0c (BioLogic Software) was used to align and double reference the data generated using the cPD1-his analyte. A dissociation constant (k_{d}) was determined from the dissociation phase data. This dissociation phase coefficient was applied as a fixed parameter in the global fit of the association phase data using a first order binding model to determine the association rate coefficient (kₐ).

To assess binding to a PD1 analyte containing the extracellular domain of human PD1 fused to a histidine tag (hPD1-his), about 8000 resonance units (RU) of the goat anti-human antibody capture antibody mentioned above were immobilized on each of three flow cells of a CMS chip as described above. Then about 350 RU of PSB103 or 600 RU of PSB205 were captured on two different flow cells coated with the goat anti-human capture antibody. The third flow cell served as a reference flow cell. The hPD1-his ligand was diluted in running buffer supplemented with 0.1% bovine serum albumin (BSA) to concentrations of 12, 25, 50, 100, 200, and 300 nM. These dilutions were injected over the three flow cells at a flow rate of 30 μL/min. Complexes were allowed to associate and dissociate for 120 and 600 seconds, respectively. The surfaces were regenerated with two 20 second injections of 10 mM glycine-HCl, pH 1.5. Duplicate injections of each analyte sample and a buffer blank were flowed over the reference and ligand-captured flow cells.

These data were manually aligned and double referenced using BIAevaluation version 4.1.1 software (General Electric Company). The data were fit to a simple 1:1 Langmuir interaction model using the global data analysis option within the software. Data for both cPD1-his and hPD1-his is shown in Table 9 below.

TABLE 9
Kinetic data for binding of cPD1
and hPD1 to PSB103 and PSB205

| Ligand | Analyte | kₐ (1/Ms) | kd (1/s) | K_{D} (nM) |
|---|---|---|---|---|
| PSB103 | cPD1-his | 1.18 × 10⁵ | 5.97 × 10⁻⁴ | 5.06 |
| PSB103 | hPD1-his | 5.22 × 10⁴ | 1.62 × 10⁻⁴ | 3.10 |
| PSB205 | hPD1-his | 5.61 × 10⁴ | 1.62 × 10⁻⁴ | 2.87 |

These data indicate that PSB103 binds to the monomeric antigens cPD1-his and hPD1-his with comparable high affinities and that PSB103 and PSB205 bind with almost the same binding kinetics to hPD1-his.

In a similar set of experiments, the kinetics of binding PSB105 to human CTLA4 (hCTLA4) and cynomolgus monkey CTLA4 (cCTLA4) and the kinetics of binding of PSB205 to hCTLA4 were assessed. A CMS sensor chip was placed on the BIAcore 3000 instrument and allowed to equilibrate overnight or longer. The running buffer was filtered and degassed prior to being placed on the system. Once the running buffer was connected, the Biacore 3000 was primed three times, and the “Normalize” system procedure was executed before running the experiment to calibrate the system's optics. All measurements occurred at 25° C. About 8000 resonance units (RU) of the goat anti-human antibody capture antibody were immobilized on each flow cell of a CM5 chip, including a flow cell for each ligand plus reference flow cell with no ligand.

About 550 RU of PSB105 was captured on a flow cell. The hCTLA4-his ligand (the extracellular domain of human CTLA4 fused to a histidine tag; AcroBiosystems catalog number CT4-H5229-100 μg) was diluted in running buffer with 0.1% BSA to 0.64, 1.25, 2.5, 5, 10, and 20 nM and injected into a flow cell at a flow rate of 30 μL/min. The cCTLA4-his ligand (the extracellular domain of cynomolgus monkey CTLA4 fused to a histidine tag; AcroBiosystems catalog number CT4-05227-200 μg) at concentrations of 3.75. 7.5, 15, 30, and 60 nM was injected into a flow cell at a rate of 30 μL/min. The complexes were allowed to associate and dissociate for 180 and 300 seconds, respectively. Surfaces were regenerated with a 40 second injection of 10 mM glycine-HCl, pH 1.5 at a flow rate of 30 μL/min. Duplicate injections of each analyte sample and a buffer blank were flowed over the reference and ligand captured surface.

As a first step in collecting information on the binding of another analyte, hCTLA4-GST-his (the extracellular domain of human CTLA4 fused to a glutathione S-transferase (GST) tag and a histidine tag, which was made in-house), to PSB105 and PSB205, about 300 RU of PSB105 and about 480 RU of PSB205 were captured on two different flow cells of a CMS chip. Another flow cell had no captured ligand and was used as a reference flow cell. The analyte was injected into the flow cells at concentrations of 12.5, 25, 50, 100, and 200 nM at a flow rate of 30 μL/min. The complexes were allowed to associate and dissociate for 120 and 600 seconds, respectively. Surfaces were regenerated with two 12 second injections of 10 mM glycine-HCl, pH 1.5. Duplicate injections of each analyte sample and a buffer blank were flowed over the reference and ligand captured surfaces.

The data from the experiments described above using CTLA4 analytes were evaluated using BIAevaluation software version 4.1.1 (General Electric Company) to manually align and double reference the data. The data were fitted to a simple 1:1 Langmuir interaction model using the global data analysis option within the software. Results are shown in Table 10 below.

TABLE 10
Kinetic data for binding of cCTLA4
and hCTLA4 to PSB105 and PSB205

| Ligand | Analyte | kₐ (1/Ms) | k_{d} (1/s) | K_{D} (nM) |
|---|---|---|---|---|
| PSB105 | cCTLA4-his | 1.73 × 10⁵ | 3.76 × 10⁻⁴ | 2.17 |
| PSB105 | hCTLA4-his | 2.35 × 10⁵ | 1.13 × 10⁻³ | 4.78 |
| PSB205 | hCTLA4-GST- | 4.53 × 10⁴ | 6.67 × 10⁻⁵ | 1.47 |
| | his | | | |
| PSB105 | hCTLA4-GST- | 4.36 × 10⁴ | 5.51 × 10⁻⁵ | 1.26 |
| | his | | | |

These data indicate that PSB105 binds to both monomeric human and cynomolgus monkey CTLA4 antigen with high affinity. Further, PSB105 and PSB205 have similar kinetics for binding of the hCTLA4-GST-his analyte. Taken together with the data in Table 9, these data indicate that each of the two antibodies in PSB205 binds to its antigens with kinetics essentially the same as those of either of these two antibodies alone.

### Example 8: Activity of PSB205 in a Cytomegalovirus (CMV) Recall Response Assay

The following experiment tested the effects of PSB205, as compared to PSB103, PSB105, or an IgG1 isotype control antibody, on numbers of CD8⁺ T cells detected in a CMV recall response assay.

An IgG1 isotype control antibody preparation was obtained from Southern Biotech (catalog number 0151k-14), and PSB103, PSB105, and PSB205 were made as described herein. Human peripheral blood mononuclear cells (PBMCs) from a CMV⁺donor were purchased from Bentech Bio (now part of Bloodworks Northwest, Seattle, Wash.).

The PBMCs were thawed and seeded into 16 wells of a microtiter plate, where each well received 3.8×10⁶ cells in a volume of 200 μL of Roswell Park Memorial Institute (RPMI) medium supplemented with 10% fetal calf serum (FCS). These cells were stimulated with a lysate of cells infected with CMV (purchased from Astarte Biologics (now Cellero), catalog number 1004, lot number 3341DE16) at a concentration of 3 μg/mL in the presence of either the IgG1 isotype control at 5 μg/mL, PSB103 at 5 μg/mL, PSB105 at 2.5 μg/mL, or PSB205 at 7.5 μg/mL. Seven days after stimulation was initiated, cells were collected and stained for 15 minutes at room temperature with 2 μL of dextramer (HLA-A*0201[NLVPMVATV]-PE purchased from Immudex, a phycoerythrin (PE)-labeled dextran/MHC class I (MHCI)/peptide conjugate that would be expected to bind to CD8⁺ T cells that specifically recognize CMV antigen pp65. Then 2 μL of an anti-CD8 antibody conjugated to fluorescein isothiocyanate (FITC) purchased from BD Bioscience was added, and staining continued for 30 minutes at room temperature. Then cells were washed three times in phosphate buffered saline (PBS), and the final cell pellet was resuspended in 400 μL of PBS supplemented with 1% bovine serum albumin (BSA). Cells were analyzed to determine numbers of cells bound by the anti-CD8 antibody and/or the dextramer using a FACscalibur™ flow cytometer (Becton Dickinson).

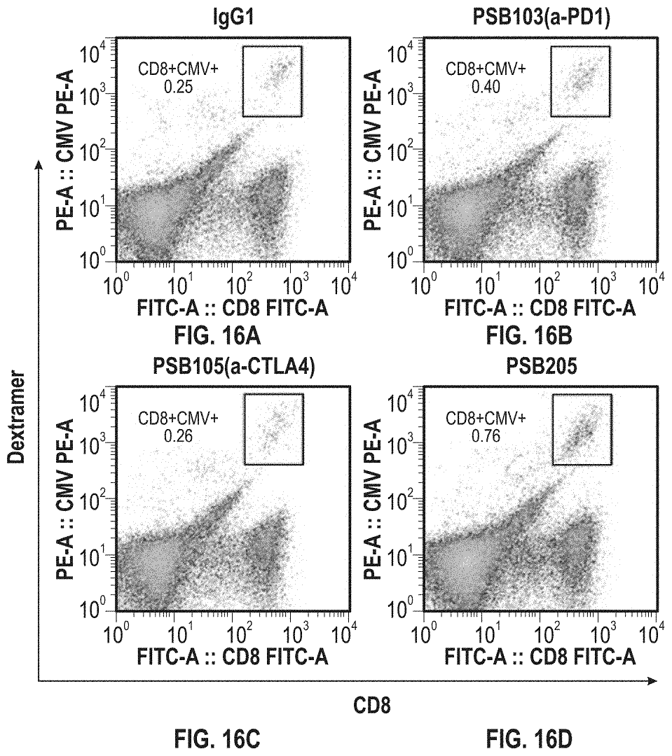

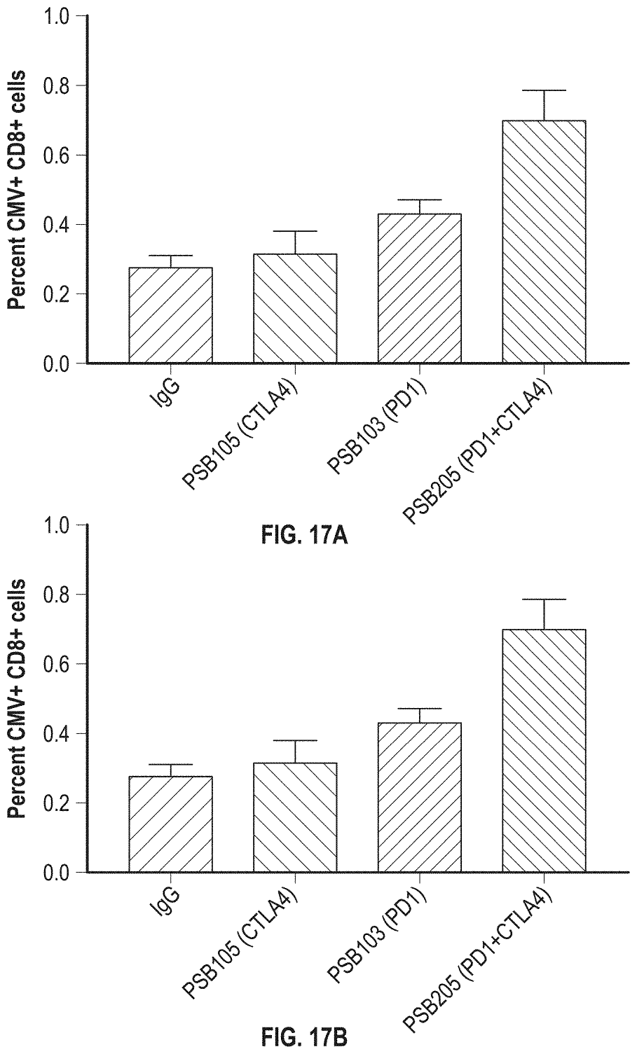

Graphs generated from these data are shown in FIG. 16. The data indicate that PBMCs stimulated in the presence of CMV lysate (antigen) and PSB205 expand more CD8⁺ cells that bind the dextramer, i.e., CD8⁺ T cells that recognize a CMV antigen (CD8⁺CMV⁺ cells), than PBMCs stimulated in the presence of CMV lystate and either PSB105 or PSB103 alone. Quantitations of (1) the absolute number of CD8⁺CMV⁺ cells and (2) the percent of all cells that are CD8⁺CMV⁺ cells indicate that PBMCs stimulated in the presence of CMV lystate plus PSB205 have more CD8⁺CMV⁺ cells than cells stimulated with CMV lysate and either PSB103 or PSB105 alone. FIG. 17. These data indicate that PSB105 has little or no effect on numbers of CD8⁺CMV⁺ cells, while PSB103 has some positive effect and PSB205 has a clearly greater positive effect than PSB103. Thus, PSB205 shows a synergistic effect (relative to PSB103 or PSB105 alone) on the expansion of CMV antigen-specific CD8⁺ T cells.

### Example 9: Efficacy of PSB205, PSB103, and PSB105 in an HCC827 Xenograft Tumor Model System

The aim of this study was to evaluate the efficacy of PSB205 and its component antibodies PSB103 and PSB105 against an established human lung adenocarcinoma cell line-derived tumor xenograft. The human adenocarcinoma cell line used to create the xenograft was HCC827. See, e.g., ATCC catalog number CRL-2868.

In more detail, each of 20 mice was inoculated subcutaneously in the right flank with 5×10⁶ HCC827 cells in 0.1 mL of PBS. The day of this inoculation is called Day 0. Study days thereafter are numbered upwards sequentially. Meanwhile, human PBMCs were isolated from peripheral blood of one healthy human donor using standard procedures and resuspended at 1×10⁸ cells/mL for implantation. When the mean tumor size reached 60-80 mm³ (about five days post-tumor inoculation), 1×10⁷ PBMCs were implanted intravenously into each mouse. Thereafter, all mice were weighed, and tumor size was measured using a caliper. Thereafter, the mice were divided into four groups for antibody treatment.

Treatment with either of four different antibody treatments (five mice per group) was started one hour after PBMC implantation. This treatment was the first dose in a course of twice per week (BIW) antibody treatments, which continued for three weeks. Antibodies were administered by intraperitoneal injection. Tumor volumes were measured twice weekly using a caliper. Details of the protocol are provided in Table 11 below.

TABLE 11
Experimental design

| | Number | | | | Antibody | |
|---|---|---|---|---|---|---|
| | of mice | HCC827 | PBMC | Antibody | dose | |
| Group | in group | inoculation | transplantation | treatment | (mg/kg) | Schedule |
| 1 | 5 | Day 0; | Injected when | Human | 7.5 | BIW for 3 |
| | | subcutaneous | tumor volume | IgG1 | | weeks |
| 2 | 5 | injection; 5 × | was 60-80 mm³; | PSB103 | 5 | |
| 3 | 5 | 10⁶ cells per | 1 × 10⁷ PBMCs | PSB105 | 2.5 | |
| 4 | 5 | mouse | per mouse | PSB205 | 7.5 | |

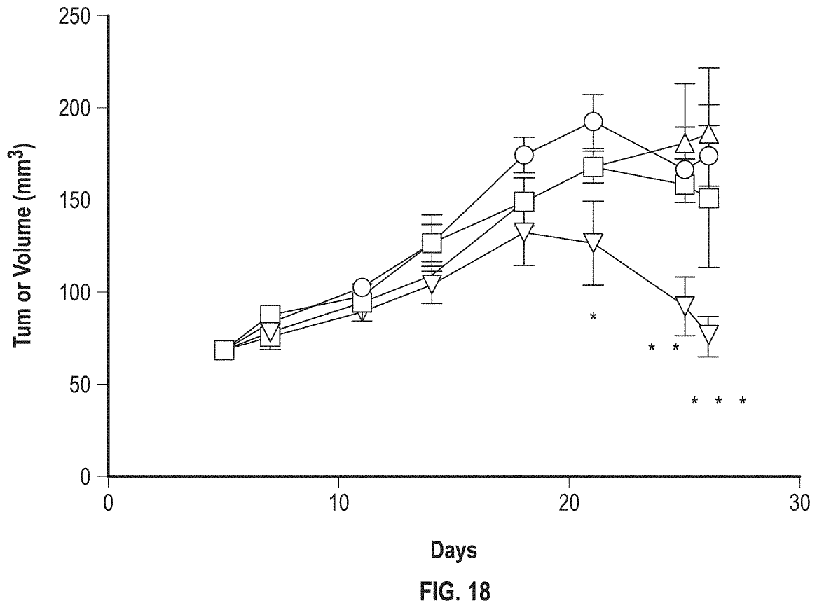

Results are shown in FIG. 18. A two-way Analysis of Variance (ANOVA) was used to analyze tumor inhibition as a function of time and treatment. These data indicated that mice in the PSB205 group showed a statistically significant tumor shrinkage compared to mice in the human IgG1 group, whereas mice in the PSB103 and PSB105 group did not. Thus, the combination of PSB103 and PSB105, i.e., PSB205, was more effective than either of these antibodies alone.

### Example 10: Preliminary Assessment of Dosing and Safety of PSB205 in Humans

An open label, dose escalation study to determine the safety of PSB205 in human patients was conducted in a center for treatment of nasopharyngeal cancer (NPC) and lung cancer (LC) patients. Dose escalation was based on an accelerated 3+3 design for doses of PSB205 from 0.3 to 10 mg/kg administered intravenously every 3 weeks (q3w). Expansion stage was carried out in selected dose cohorts. The primary objective of the study was to define the safety and tolerability of PSB205 by determining the maximum tolerated dose (MTD) and a recommended phase 2 dose (RP2D) of PSB205 in patients with advanced malignant tumors.

Forty-four NPC and LC patients were enrolled. The diameter(s) one or more tumors (up to a maximum of five tumors) from each patient was measured using a computed tomography scan (CT scan) at baseline and at weeks 7, 13, 22, and 31 thereafter. If more than one tumor was measured, a sum of the diameters of the measured tumors was determined. This sum is referred to as the sum of the target lesions. Patients were free to discontinue their participation in the study at any time. Table 12 below summarizes the enrollment status and preliminary efficacy data for the response evaluable subjects. To be “response evaluable” a patient had to have had at least the week 7 CT scan to determine whether her tumor(s) had shrunk, grown, or remained the same following treatment with PSB205.

TABLE 12
Subjects, dosing, and preliminary response data.

| | | | | | | | | Sum of the target |
|---|---|---|---|---|---|---|---|---|
| | | | | | | | | lesions at |
| | | | | | | | | baseline/week 7 |
| | | | | Prior | Date of | | | response⁹/week 13 |
| | | | | anti- | first dose | | | response/week 22 |
| | Subject | Dose | Tumor | hPD1 | (DD/MM/ | Cycle | Subject | response/week 31 |
| # | # | (mg/kg) | type | treatment ⁴ | YYYY) | #⁵ | status | response |
| 1 | 01001 | 0.3 | LC² | | 31/03/2020 | 2 | Disctd⁶ | 32/PD |
| 2 | 01002 | 1 | NPC³ | | 22/04/2020 | 11 | | 33/PR/PR/PR/PR |
| 3 | 01003 | 1 | LC | nivolumab | 23/04/2020 | 2 | Disctd | 53/PD |
| 4 | 01004 | 1 | LC | | 23/04/2020 | 11 | | 50/SD/SD/SD/PR |
| 5 | 01008 | 1-PK¹ | LC | | 03/06/2020 | 9 | | 57/SD/SD/PD |
| 6 | 01009 | 1-PK | NPC | | 05/06/2020 | 2 | Disctd | 95/PD |
| 7 | 01013 | 1-PK | NPC | camrelizumab/ | 19/06/2020 | 2 | Disctd | 71/PD) |
| | | | | placebo | | | | |
| 8 | 01005 | 3 | LC | | 15/05/2020 | 8 | Disctd | 52/SD/SD/SD/PD |
| 9 | 01006 | 3 | NPC | camrelizumab/ | 20/05/2020 | 4 | Disctd | 29/PD/PD |
| | | | | placebo | | | | |
| 10 | 01007 | 3 | NPC | camrelizumab/ | 27/05/2020 | 4 | Disctd | 21/PD/PD |
| | | | | placebo | | | | |
| 11 | 01015 | 3-PK | NPC | camrelizumab/ | 03/07/2020 | 8 | | 13/SD/SD/SD |
| | | | | placebo | | | | |
| 12 | 01025 | 3-PK | LC | | 14/08/2020 | 6 | | 138/PR/PR |
| 13 | 01028 | 3-PK | LC | Keytruda ® | 28/08/2020 | 3 | Disctd | 59/PD |
| 14 | 01010 | 5 | NPC | | 19/06/2020 | 8 | | 64/PR/PR/PR |
| 15 | 01011 | 5 | NPC | | 23/06/2020 | 8 | | 89/PR/PR/PR |
| 16 | 01014 | 5 | NPC | camrelizumab/ | 22/06/2020 | 8 | | 50/SD/PR/PR |
| | | | | placebo | | | | |
| 17 | 01016 | 5-PK | NPC | camrelizumab/ | 17/07/2020 | 2 | Disctd | 80/PD |
| | | | | placebo | | | | |
| 18 | 01017 | 5-PK | NPC | camrelizumab/ | 31/07/2020 | 2 | Disctd | 128/PD |
| | | | | placebo | | | | |
| 19 | 01018 | 5-PK | LC | | 22/07/2020 | 7 | | 49/SD/SD/PD |
| 20 | 01020 | 5-PK | NPC | camrelizumab/ | 30/07/2020 | 2 | Disctd | 45/PD |
| | | | | placebo | | | | |
| 21 | 01023 | 5-PK | NPC | toripalimab | 14/08/2020 | 2 | Disctd | 142/PD |
| 22 | 01024 | 5-PK | LC | Keytruda ® | 14/08/2020 | 2 | Disctd | 43/PD |
| 23 | 01029 | 5-PK | NPC | | 09/09/2020 | 2 | Disctd | 86/PD |
| 24 | 01031 | 5-PK | LC | | 11/09/2020 | 2 | Disctd | 143/PD |
| 25 | 01033 | 5-PK | LC | | 23/09/2020 | 4 | | 63/SD |
| 26 | 01038 | 5-PK | NPC | camrelizumab/ | 21/10/2020 | 2 | | 57/PD |
| | | | | placebo | | | | |
| 27 | 01040 | 5-PK | NPC | | 28/10/2020 | 3 | | 101/PR |
| 28 | 01041 | 5-PK | NPC | | 12/11/2020 | 2 | | 44/PR |
| 29 | 01042 | 5-PK | NPC | JS001/ | 04/11/2020 | 2 | | 38/SD |
| | | | | placebo | | | | |
| 30 | 01026 | 10 | LC | sintilimab | 27/08/2020 | 2 | Disctd | 57/PD |
| | | | | (IBI308)/ | | | | |
| | | | | placebo | | | | |
| 31 | 01027 | 10 | LC | nivolumab | 28/08/2020 | 5 | | 77/SD/PR |
| 32 | 01030 | 10 | NPC | | 02/09/2020 | 1 | Disctd⁷ | |
| 33 | 01035 | 10 | NPC | toripalimab | 13/10/2020 | 2 | Disctd⁸ | |
| 34 | 01036 | 10 | LC | | 14/10/2020 | 1 | Disctd⁷ | |
| 35 | 01037 | 10 | NPC | camrelizumab/ | 21/10/2020 | 2 | | 93/PR |
| | | | | placebo | | | | |

¹Dose indications followed by “-PK” indicate patients in which pharmacokinetic measurements were taken, in addition to monitoring safety and efficacy indices.
²“LC” stands for lung cancer
³“NPC” stands for nasopharyngeal cancer.
⁴ A blank box indicates that there was no prior anti-hPD1 targeted treatment. Patients described as having had a prior treatment that was either of two treatments, e.g., “camrelizumab/placebo”, had been in a blinded clinical trial in which they did not know whether they had received drug or placebo.
⁵The “cycle #” indicates the number of times the patient was dosed with PSB205.
⁶“Disctd” stands for discontinued.
⁷These subjects discontinued study treatment due to a dose limiting toxicity (DLT).
⁸This subject discontinued study treatment due to a grade 4 infusion reaction.
⁹Response is indicated as progressive disease (PD), partial response (PR), stable disease (SD), or complete response (CR) as defined herein above, consistent with RECIST guidelines (version 1.1).

To highlight the observed efficacy of PSB205 suggested by the results in this trial, the available preliminary data is summarized in Table 13, which shows the Disease Control Rate (DCR, i.e., the percentage of evaluable subjects that exhibited partial or complete response (PR or CR) or stable disease (SD)), as well as the Objective Response Rate (ORR, i.e., the percentage of patients showing a PR or a CR after treatment).

TABLE 13
Preliminary efficacy data

| | Subjects | | Partial | | | | | | | |
|---|---|---|---|---|---|---|---|---|---|---|
| Dose | (n) | | response | | Stable disease | | | | DCR | ORR |
| (mg/kg) | LC¹ | NPC² | LC | NPC | LC | NPC | DCR | ORR | (LC) | (NPC) |
| 0.3 | 1 | 0 | 0 | 0 | 0 | 0 | 0% | 0% | 0% | 0% |
| 1 | 3 | 3 | 1 | 1 | 1 | 0 | 50% | 33% | 67% | 33% |
| 3 | 3 | 3 | 1 | 0 | 1 | 1 | 50% | 17% | 67% | 0% |
| 5 | 4 | 12 | 0 | 5 | 2 | 1 | 50% | 31% | 50% | 42% |
| 10 | 2 | 1 | 1 | 1 | 0 | 0 | 67% | 67% | 50% | 100% |
| Total | 13 | 19 | 3 | 7 | 4 | 2 | | | | |
| DCR | 54% | 47% | | | | | | | | |
| ORR | 23% | 37% | | | | | | | | |

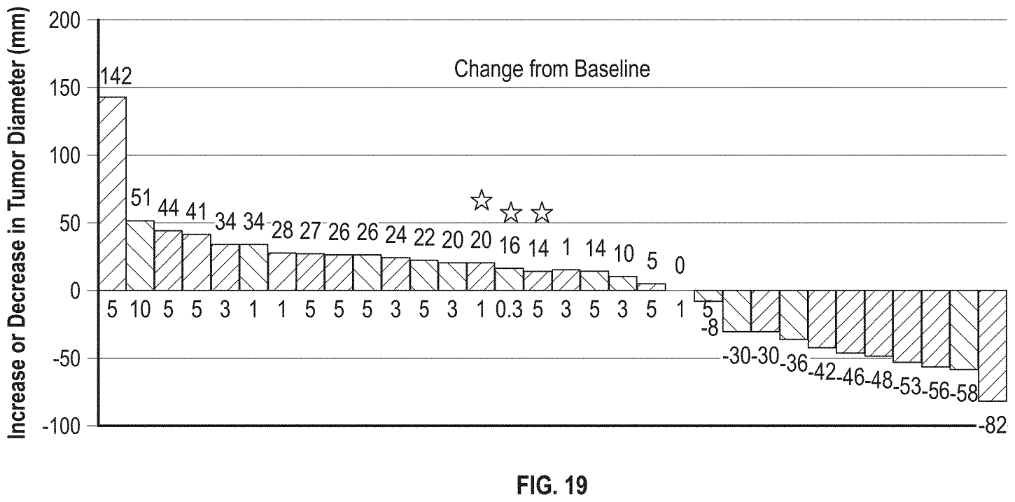

The collective data at doses from 1-10 mg/kg suggest that these doses of PSB205 may be effective treatment doses for LC and NPC. To illustrate the extent of the observed responses, FIG. 19 shows the change in tumor diameter from baseline for each of the 32 evaluable subjects, along with the dose each received. These data indicate that substantial responses were achieved by some patients receiving doses of PSB205 from 1-10 mg/kg.

Table 14 provides data on the adverse events (AEs) experienced by the patients dosed with 1 or 3 mg/kg PSB205. Grades 1, 2, 3, and 4 AEs are defined as stated above and in Common Terminology Criteria for Adverse Events (CTCAE) version 5.0 2010, available at //ctep.cancer/gov/protocoldevelopment/electronic_applications/doc/CTCAE_v5_Quick_Reference)8.5x11.pdf, which is incorporated here by reference). No AEs were observed in patients treated with 0.3 mg/kg.

TABLE 14
Summary of adverse events at doses of 1 and 3 mg/kg PSB205

| | 1.0 mg/kg | | | | | 3.0 mg/kg | | | |
|---|---|---|---|---|---|---|---|---|---|
| | (n = 6) | | | | | (n = 6) | | | |
| | | | | | Grade 3 | | | | Grade 3 |
| Description | Grade 1 | | Grade 2 | | or 4 | Grade 1 | | Grade 2 | or 4 |
| of AE | n(%) | | n(%) | | n(%) | n(%) | | n(%) | n(%) |
| Any treatment | 3 | (50%) | 2 | (33%) | 0 | 1 | (16.7%) | 1 (16.7%) | 0 |
| emergent AE | | | | | | | | | |
| (TEAE) | | | | | | | | | |

| | | | | | | | | |
|---|---|---|---|---|---|---|---|---|
| Pruritus | 2 | (33%) | 0 | 0 | 1 | (16.7%) | 1 (16.7%) | 0 |
| Rash | 2 | (33%) | 0 | 0 | 2 | (33%) | 0 | 0 |

| | | | | | | | |
|---|---|---|---|---|---|---|---|
| Aspartate | 1 | (16.7%) | 0 | 0 | 0 | 0 | 0 |
| aminotransferase | | | | | | | |
| increased | | | | | | | |

| | | | | | | | |
|---|---|---|---|---|---|---|---|
| Alanine | 0 | 1 | (16.7%) | 0 | 0 | 0 | 0 |
| aminotransferase | | | | | | | |
| increased | | | | | | | |

| | | | | | | | |
|---|---|---|---|---|---|---|---|
| Fatigue | 1 | (16.7%) | 0 | 0 | 0 | 0 | 0 |

| | | | | | | | | |
|---|---|---|---|---|---|---|---|---|
| Hypothyroidism | 0 | 2 | (33%) | 0 | 1 | (16.7%) | 0 | 0 |

| | | | | | | | |
|---|---|---|---|---|---|---|---|
| Hyperthyroidism | 0 | 1 | (16.7%) | 0 | 0 | 0 | 0 |

| Infusion-related | 0 | | 0 | 0 | 0 | 0 | 0 |
|---|---|---|---|---|---|---|---|
| reaction | | | | | | | |
| Pyrexia | 0 | | 0 | 0 | 0 | 0 | 0 |
| Asthenia | 1 | (16.7%) | 0 | 0 | 0 | 0 | 0 |
| Platelet count | 0 | | 0 | 0 | 0 | 0 | 0 |
| decreased | | | | | | | |
| Arthralgia | 1 | (16.7%) | 0 | 0 | 0 | 0 | 0 |
| Autoimmune | 0 | | 0 | 0 | 0 | 0 | 0 |
| nephritis | | | | | | | |
| Bilirubin | 0 | | 0 | 0 | 0 | 0 | 0 |
| conjugated | | | | | | | |
| increased | | | | | | | |
| Blood bilirubin | 0 | | 0 | 0 | 0 | 0 | 0 |
| increased | | | | | | | |
| Decreased | 1 | (16.7%) | 0 | 0 | 0 | 0 | 0 |
| appetite | | | | | | | |
| Dermatitis | 0 | | 0 | 0 | 0 | 0 | 0 |
| Gingival | 0 | | 0 | 0 | 0 | 0 | 0 |
| bleeding | | | | | | | |
| Hyponatraemia | 0 | | 0 | 0 | 0 | 0 | 0 |
| Oedema | 1 | (16.7%) | 0 | 0 | 0 | 0 | 0 |
| peripheral | | | | | | | |

| Total bile acids | 0 | 0 | 0 | 0 | | 0 | 0 |
|---|---|---|---|---|---|---|---|
| increased | | | | | | | |
| Vomiting | 0 | 0 | 0 | 1 | (16.7%) | 0 | 0 |

| | | | | | | |
|---|---|---|---|---|---|---|
| Weight | 0 | 0 | 0 | 0 | 1 (16.7%) | 0 |
| decreased | | | | | | |

Table 15 shows data on AEs observed in patients dosed with 5 or 10 mg/kg PSB205.

TABLE 15
Summary of adverse events at doses of 5 and 10 mg/kg PSB205

| | 5.0 mg/kg | | | | | 10.0 mg/kg | | | |
|---|---|---|---|---|---|---|---|---|---|
| | (n = 22) | | | | | (n = 6) | | | |
| | | | | | Grade 3 | | | Grade 3 | |
| Description | Grade 1 | | Grade 2 | | or 4 | Grade 1 | Grade 2 | or 4 | |
| of AE | n(%) | | n(%) | | n(%) | n(%) | n(%) | n(%) | |
| Any TEAE | 9 | (40.9%) | 3 | (13.6%) | 1 (4.5%) | 2 (33.3%) | 1 (16.7%) | 3 | (50%) |
| Pruritus | 4 | (18.2%) | 0 | | 0 | 1 (16.7%) | 0 | 0 | |
| Rash | 3 | (13.6%) | 1 | (4.5%) | 0 | 1 (16.7%) | 0 | 0 | |
| Aspartate | 5 | (22.7%) | 0 | | 1 (4.5%) | 0 | 0 | 0 | |
| aminotransferase | | | | | | | | | |
| increased | | | | | | | | | |
| Alanine | 0 | | 1 | (4.5%) | 0 | 2 (33.3%) | 0 | 0 | |
| aminotransferase | | | | | | | | | |
| increased | | | | | | | | | |
| Fatigue | 2 | (9.1%) | 0 | | 0 | 0 | 1 (16.7%) | 0 | |
| Hypothyroidism | 0 | | 1 | (4.5%) | 0 | 0 | 0 | 0 | |
| Hyperthyroidism | 2 | (9.1%) | 0 | | 0 | 0 | 0 | 0 | |
| Infusion-related | 1 | (4.5%) | 1 | (4.5%) | 0 | 0 | 0 | 1 | (16.7%) |
| reaction | | | | | | | | | |

| | | | | | | | |
|---|---|---|---|---|---|---|---|
| Pyrexia | 1 | (4.5%) | 0 | 0 | 1 (16.7%) | 1 (16.7%) | 0 |
| Asthenia | 1 | (4.5%) | 0 | 0 | 0 | 0 | 0 |

| Platelet count | 0 | 0 | 0 | 0 | 0 | 2 | (33.3%) |
|---|---|---|---|---|---|---|---|
| decreased | | | | | | | |
| Arthralgia | 0 | 0 | 0 | 0 | 0 | 0 | |
| Autoimmune | 0 | 0 | 0 | 0 | 0 | 1 | (16.7%) |
| nephritis | | | | | | | |

| Bilirubin | 0 | | 0 | 0 | 1 (16.7%) | 0 | 0 |
|---|---|---|---|---|---|---|---|
| conjugated | | | | | | | |
| increased | | | | | | | |
| Blood bilirubin | 0 | | 0 | 0 | 0 | 1 (16.7%) | 0 |
| increased | | | | | | | |
| Decreased | 0 | | 0 | 0 | 0 | 0 | 0 |
| appetite | | | | | | | |
| Dermatitis | 1 | (4.5%) | 0 | 0 | 0 | 0 | 0 |

| | | | | | | |
|---|---|---|---|---|---|---|
| Gingival | 0 | 0 | 0 | 1 (16.7%) | 0 | 0 |
| bleeding | | | | | | |
| Hyponatraemia | 0 | 0 | 0 | 1 (16.7%) | 0 | 0 |
| Oedema | 0 | 0 | 0 | 0 | 0 | 0 |
| peripheral | | | | | | |
| Total bile acids | 0 | 0 | 0 | 0 | 1 (16.7%) | 0 |
| increased | | | | | | |
| Vomiting | 0 | 0 | 0 | 0 | 0 | 0 |
| Weight decreased | 0 | 0 | 0 | 0 | 0 | 0 |

Table 16 summarizes data from Tables 14 and 15.

TABLE 16
Summary of all AEs at doses from 1-10 mg/kg

| | All doses from 1-10 mg/kg | | | | | | | |
|---|---|---|---|---|---|---|---|---|
| | (n = 40) | | | | | | | |
| Description | Grade 1 | | Grade 2 | | Grade 3 or 4 | | All grades | |
| of AE | n (%) | | n (%) | | n (%) | | n (%) | |
| Any TEAE | 15 | (37.5%) | 6 | (15%) | 4 | (10%) | 25 | (62.5%) |
| Pruritus | 8 | (20%) | 1 | (2.5%) | 0 | (0%) | 9 | |
| Rash | 8 | (20%) | 1 | (2.5%) | 0 | (0%) | 9 | |
| Aspartate | 6 | (15%) | 0 | (0%) | 1 | (2.5%) | 7 | |
| amino- | | | | | | | | |
| transferase | | | | | | | | |
| increased | | | | | | | | |
| Alanine | 2 | (5%) | 2 | (5%) | 0 | (0%) | 4 | (10%) |
| amino- | | | | | | | | |
| transferase | | | | | | | | |
| increased | | | | | | | | |
| Fatigue | 3 | (7.5%) | 1 | (2.5%) | 0 | (0%) | 4 | (10%) |
| Hypo- | 1 | (2.5%) | 3 | (7.5%) | 0 | (0%) | 4 | (10%) |
| thyroidism | | | | | | | | |
| Hyper- | 2 | (5%) | 1 | (2.5%) | 0 | (0%) | 3 | (7.5%) |
| thyroidism | | | | | | | | |
| Infusion-related | 1 | (2.5%) | 1 | (2.5%) | 1 | (2.5%) | 3 | (7.5%) |
| reaction | | | | | | | | |
| Pyrexia | 2 | (5%) | 1 | (2.5%) | 0 | (0%) | 3 | (7.5%) |
| Asthenia | 2 | (5%) | 0 | (0%) | 0 | (0%) | 2 | (5%) |
| Platelet count | 0 | (0%) | 0 | (0%) | 2 | (5%) | 2 | (5%) |
| decreased | | | | | | | | |
| Arthralgia | 1 | (2.5%) | 0 | (0%) | 0 | (0%) | 1 | (2.5%) |
| Autoimmune | 0 | (0%) | 0 | (0%) | 1 | (2.5%) | 1 | (2.5%) |
| nephritis | | | | | | | | |
| Bilirubin | 1 | (2.5%) | 0 | (0%) | 0 | (0%) | 1 | (2.5%) |
| conjugated | | | | | | | | |
| increased | | | | | | | | |
| Blood bilirubin | 0 | (0%) | 1 | (2.5%) | 0 | (0%) | 1 | (2.5%) |
| increased | | | | | | | | |
| Decreased | 1 | (2.5%) | 0 | (0%) | 0 | (0%) | 1 | (2.5%) |
| appetite | | | | | | | | |
| Dermatitis | 1 | (2.5%) | 0 | (0%) | 0 | (0%) | 1 | (2.5%) |
| Gingival | 1 | (2.5%) | 0 | (0%) | 0 | (0%) | 1 | (2.5%) |
| bleeding | | | | | | | | |
| Hyponatraemia | 1 | (2.5%) | 0 | (0%) | 0 | (0%) | 1 | (2.5%) |
| Oedema | 1 | (2.5%) | 0 | (0%) | 0 | (0%) | 1 | (2.5%) |
| peripheral | | | | | | | | |
| Total bile acids | 0 | (0%) | 1 | (2.5%) | 0 | (0%) | 1 | (2.5%) |
| increased | | | | | | | | |
| Vomiting | 1 | (2.5%) | 0 | (0%) | 0 | (0%) | 1 | (2.5%) |
| Weight | 0 | (0%) | 1 | (2.5%) | 0 | (0%) | 1 | (2.5%) |
| decreased | | | | | | | | |

The data in Tables 14-16 show that no grade 3 or 4 AEs were observed in the 1 mg/kg or 3 mg/kg dosage groups. In the 5 mg/kg group, only one out of 22 patients (4.5%) experienced a grade 3 or 4 event. At 10 mg/kg, three out of six patients (50%) experienced a grade 3 or 4 event. The most common AEs include grade 1 pruritus, rash, and increases in aspartate aminotransferase (which can indicate malfunction of, e.g., the liver, heart, or other organs).

### Example 11: Supplemental Assessment of Dosing and Safety of PSB205 in Humans

The following Example 11, including FIGS. 20-32, Tables 17-26, and their legends, provides additional data and analysis on various aspects of what is described above and is meant to supplement, but not limit the scope of, what is described above.

### Design and Generation of PSB205

A recombinant antibody is typically produced by a single engineered cell line in which the heavy chain (HC) and light chain (LC) of the antibody need to be correctly assembled before it can be secreted. In order to produce two antibodies together, two different HCs and LCs need to be introduced in the same host cell. Due to random pairing of HC and LC, total of ten products can be generated with only two of them having the cognate chain pairings (FIG. 20A). We specifically made changes in the HC/HC and HC/LC interface residues in such a way that the correct assemble of cognate HC/HC and HC/LC pairing is strongly favored. When these uniquely designed HC pairing keys and LC pairing keys were introduced, the two different antibodies can be expressed together without any mis-paired species. The product generated by this antibody engineering technology platform contains the mixture of two recombinant antibodies in a fixed ratio, and is designated as a MabPair molecule. PSB205 is a MabPair product developed to target PD-1 and CTLA-4, two key immune checkpoint regulators.

Anti-PD-1 and anti-CTLA4 antibodies were individually engineered and characterized (Table 20-21). Anti-PD-1 IgG4, designated as PSB103, was shown to possess a potency similar to pembrolizumab in multiple cell-based assays (FIG. 24, Table 20). Anti-CTLA-4 IgG1, designated as PSB105, exhibited blocking activities similar to ipilimumab. A single mutation at arginine 255 (R255K) was introduced in the Fc region to reduce the binding to FcRn (Table 22), leading to a faster clearance and shortening of antibody half-life in vivo for the anti-CTLA-4 compared to ipilimumab (Table 23).

PSB103 (anti-PD-1 IgG4) and PSB105 (anti-CTLA-4 IgG1) were produced together in a CHO cell line in a fixed ratio of 2:1 (FIG. 20B). The relative ratio of anti-PD-1 and anti-CTLA-4 antibodies in PSB205 was determined by using allometrically scaled PK simulations for its components. The simulation predicts that when PSB205 is dosed at three-week intervals, it will achieve a different level of steady state exposure for the anti-PD-1 and anti-CTLA-4 antibodies. PSB205 was manufactured as a single product, and its purity and product quality were fully characterized by using a panel of analytical methods. No detectable mis-pairing species were found in the product (FIG. 20C-E).

Preclinical characterization of PSB205

The ability of PSB205 and its anti-PD-1 component (PSB103) or anti-CTLA4 component (PSB105) to block the PD-1:PD-L1 interaction or CTLA-4:B7-1/B7-2 interaction was evaluated using two different dual-cell reporter assays. As displayed in FIG. 25, both PSB103 and PSB205 mediated concentration-dependent inhibition of the PD-L1:PD-1 interaction that enabled NFAT activation and an increased luciferase signal. PSB105 and PSB205 also released CTLA4 mediated inhibition in the reporter assay.

Dendritic cells express costimulatory (B7-1 and B7-2) and coinhibitory (PD-L1) molecules to engage CD28/CTLA4 and PD-1 expressed by T cells, respectively. To determine how PSB205 affects T cell stimulation by dendritic cells, immature dendritic cells derived from the monocytes of an alloreactive donor were used to stimulate purified T cells. As shown in FIG. 21A, both PSB103 alone and PSB205 increased interferon-γ (IFN-γ) production by T cells at different DC/T ratios. anti-CTLA-4 did not contribute to the increase of IFN-γ production.

Since PSB103 and PSB105 have different PK profiles, their ratio in human will change in time. To assess the range of synergistic effect of PSB205, SEB in the presence of various concentrations of PSB103 and PBS 105 mixed at different ratios were used to stimulate peripheral blood mononuclear cell (PBMC). As depicted in FIG. 21B and FIG. 26, the combination of PSB103 (ranging from 0.5 to 20 μg/mL) and PSB105 (ranging from 0.05 to 4 μg/mL) at different ratios (ranging from 5:1 to 0.2:1) increased interleukin 2 (IL-2) production by T cells, suggesting that PSB103 and PB S105 in PB S205 can achieve a broad range of a synergistic effect in vivo during the treatment.

To determine whether the combination of PSB103 and PSB105 at the ratio of 2:1 in PSB205 can achieve a synergistic effect in stimulating antigen specific CD8 T cells, cytomegalovirus (CMV) lysate from CMV infected cells was used to stimulate PBMCs from a seropositive individual. We enumerated the expansion of CD8 T cell specific for pp65 of CMV on day 7 using dextramer from immudex HLA-A*0201/NLVPMVATV. As shown in FIG. 21C and FIG. 27, higher percentages and absolute numbers of CMV+CD8 T cells were recovered from PSB205 treated group than the group that had been treated with either PSB103 (anti-PD1) or PSB105 (anti-CTLA4) alone.

PSB205 was evaluated in a tumor xenograft model in NOD-Prkdcscid IL2Rγ null (NCG) mouse containing human immune cells derived from PBMCs. As shown in FIG. 21D, the combination of PSB103 and PSB 105 at 2:1 ratio (PSB205) was effective in controlling HCC827 tumor growth, whereas either PSB103 or PSB105 alone did not show any effect in this model. We further tested PSB205 in Jeko-1 tumor model where the tumor grows faster than HCC827. As shown in FIG. 28, either PSB103 or PSB105 alone significantly inhibited Jeko-1 tumor growth, but PSB205 was more effective than PSB103 alone.

The PK profiles of PSB103 and PSB105 were evaluated individually in single-dose exploratory experiments in cynomolgus monkeys. Systemic exposure was achieved in all animals following a single i.v. injection. The average terminal elimination half-life (t_{1/2}) was determined to be 297 hours for PSB103. (Table 23). PSB105 showed an increased rate of clearance and reduced systemic exposure as compared to ipilimumab. t_{1/2} of PSB105 and ipilimumab were 109 and 397 hours, respectively (FIG. 21E). This is at least partially attributed to the reduced FcRn affinity in PSB 105.

PSB205 was evaluated in a multi-dose GLP toxicity experiment. Injection of PSB205 to cynomolgus monkeys every 2 weeks for over 4 weeks (Days 1, 15, and 29) at 3, 15 or 60 mg/kg, respectively, was well tolerated and did not result in any gross adverse events that were related to PSB205. Loose stools were observed for a few animals at 60 mg/kg, but no pathological changes were identified. PSB205 treatment led to an increase of Ki67+ T cells and ICOS+CD4 T cells, which is a unique biomarker for CTLA-4 blockade. The increase of ICOS+CD4+ T cells was directly related to the dose of PSB205 (FIG. 29).

### Phase 1 Clinical Trial of PSB205 (QL1706)

### Baseline Characteristics

A total of 47 patients with solid tumors between March 31 and Dec. 20, 2020, were enrolled, with 16 and 31 patients in the dose-escalation and expansion cohorts, respectively. The baseline characteristics of the included patients were shown in Table 17. The median age was 51 years (ranging from 27 to 73 year) and 38 (80.9%) was males. Seventeen (36.2%) patients had an ECOG performance status of 0. Of the patients, 25 (53.2%) had nasopharyngeal carcinoma (NPC), 20 (42.6%) had NSCLC, one (2.1%) had thyroid cancer and one (2.1%) had mucinous adenocarcinoma of the umbilical canal. Forty-six (97.9%) patients were in stage IV and only one in stage III. Twenty-eight (59.6%) patients had no history of immunotherapy, 18 (38.3%) had received ICI treatments and 4 (8.5%) received other immunotherapy. The median of prior lines of therapy was 2.0 (ranging 0-5).

### Pharmacokinetic Analysis

The PK profiles of anti-PD-1 and anti-CTLA-4 components of PSB205 were characterized separately by using two different anti-idiotypic antibodies that are specific to each component. The analysis was carried out with data collected from Cycle 1 (N=36) and Cycle 6 (N=9) of the treatment.

FIG. 22A-B illustrates the mean concentration-time profiles for aPD-1 and aCTLA-4 following administration of 0.3 to 10 mg/kg of PSB205 every 3 weeks (Q3W). The exposure of both aCTLA-4 and aPD-1 increased as the dose increased following single- and multiple-dose administration. As illustrated in FIG. 30A-D, the distributions of individual dose-normalized Cₘₐₓ and AUC₀₋₄ for aCTLA-4 and aPD-1 among different doses were similar, indicating that both aCTLA-4 and aPD-1 might exhibit linear PK characteristics at single doses ranging from 0.3 to 10 mg/kg. Given the limited number of patients with available multiple-dose data, the linearity of PK characteristics of aCTLA-4 and aPD-1 after multiple-dose administration were not established at this time. The PK parameters of aCTLA-4 and aPD-1 are summarized in Table 24.

The clearance (CL) of aCTLA-4 remained similar following single- and multiple-dose administration (i. e. 0.0159-0.0252 L/h and 0.0134-0.0225 L/h, respectively). The corresponding mean t_{1/2} were 104-121 h (4-5 days) and 111-190 h (5-8 days), respectively. No significant accumulation of aCTLA-4 was observed following multiple dosing.

The mean CL of aPD-1 was 0.0122-0.0159 L/h following single dose administration (n=10), which decreased to 0.00676-0.00720 L/h following multiple-dose administration (n=5, Cycle 6). The mean t_{1/2} were 147-227 h (6-9 days) following single dose administration (n=10), but could not be estimated accurately following multiple dose administration due to limited sampling time points. Nonetheless, much longer t_{1/2} would be expected at the steady state. As shown in Table 24, a certain accumulation of aPD-1 might exist after repeated Q3W administration of PSB205. The mean R_{ac_}C_{trough} (accumulation ratio assessed by trough concentration [C_{trough}]), R_{ac_}Cₘₐₓ (accumulation ratio assessed by Cₘₐₓ), and R_{ac_}AUC (accumulation ratio assessed by AUC₀₋ₜₐᵤ) were 1.79-2.40 (n=8), 1.24-1.59 (n=9), and 1.52-1.77 (n=4), respectively.

### Pharmacodynamics

The level of PD-1 target coverage by PSB205 was assessed by receptor occupancy assay on circulating CD3 T cells. A sustained high percentage of PD-1 receptor occupancy rate was observed in all dosing groups throughout the treatment cycle (FIG. 22C). No dose dependent difference in receptor occupancy was observed. The fluctuation in receptor occupancy shown in some patients taking 10 mg/kg was likely due to the smaller sample size and individual variation. PSB205 administration was associated with enhanced proliferation of both CD4 and CD8 cells. As depicted in FIG. 22D, the increase of KI67+ cells in CD4 and CD8 T cell population was more significant in the 5 mg/kg and 10 mg/kg group than in the lower dose groups. In addition, there was a dose dependent upregulation of ICOS on CD4 T cells, a well-recognized surrogate for CTLA-4 blockades. The highest increase of ICOS+CD4 T cells over the baseline was observed in 5 mg/kg and 10 mg/kg group (FIG. 22E, FIG. 31). The increase of ICOS+CD4 T cells was sustained for at least two weeks (336 hrs) in the 5 mg/kg and 10 mg/kg groups, but not in the 1 mg and 3 mg/kg groups (FIG. 32). The consistent and sustained increase of Ki67+ T cells and ICOS+CD4 T cells suggest functional blockade of PD-1 and CTLA-4 can be achieved at dose higher than 5 mg/kg.

### Safety Data

Patients received a median of 2 cycles (1-12) of PSB205 administered every 3 weeks. As of Dec. 20, 2020, the median follow-up was 54 days (16, 128), and 25 of 47 (53.2%) patients were still on treatment. Among the 22 patients who discontinued treatment, the most common reason was disease progression (n=15, 68.2%), followed by AEs (n=5, 22.7%) and withdrawal from the study (n=2, 9.1%).

Treatment related adverse events (TRAEs) occurred in 31 (66.0%) of the 47 patients, with the frequencies of 83.3% (5/6), 33.3 (2/6), 67.8 (19/28) and 83.3% (5/6), respectively, in the 1 mg/kg, 3 mg/kg 5 mg/kg, and 10 mg/kg groups (Table 18). Most patients experienced grade 1 TRAEs (38.3%, 18/47), especially in those who receiving 5 mg/kg (50%, 14/28). Two patients receiving 5 mg/kg and three patients receiving 10 mg/kg experienced TRAEs with a grade ≥3. Overall, the most common (≥5%) TRAEs were pruritus (23.4%, 11/47), rash (21.3%, 10/47), AST increased (14.9%, 7/47), fatigue (12.8%, 6/47), hyperthyroidism (10.6%, 5/47), hypothyroidism (10.6%, 5/47), ALT increased (8.5%, 4/47), pyrexia (8.5%, 4/47), and infusion related reaction (6.4%, 3/47).

irAEs of any grades occurred in 16 (34.0%) of 47 patients. The most common (≥5%) irAEs were pruritus (23.4%, 11/47), rash (21.3%, 10/47), hyperthyroidism (10.6%, 5/47), and hypothyroidism (10.6%, 5/47) (Table 25). Two (4.3%) patients, one in the 5 mg/kg group and one in the 10 mg/kg group, had an irAE with a grade ≥3.

Serious adverse events (SAE) regardless of causes occurred in seven (14.9%) of 47 patients, and six were considered drug-related, and occurred in 5 patients, one receiving 5 mg/kg and four receiving 10 mg/kg.

Two patients in the 10 mg/kg group experienced dose-limiting toxicity (DLT), including one case with grade 3 decreased platelet count complicated with grade 1 gingival bleeding and one case with grade 4 immune-mediated nephritis. Thus, the maximum tolerated dose (MTD) was determined as 5 mg/kg Q3W.

### Clinical Efficacy

As shown in Table 19, of 35 patients with available data for efficacy analysis, 10 (28.6%) had a partial response (PR), and 7 (20.0%) had a stable disease (SD), resulting in an objective response rate (ORR) of 28.6% and a disease control rate (DCR) of 48.5%. In 20 NPC patients, 7 (35.0%) achieved PR and 2 (10.0%) achieved SD, with an ORR of 35.5% and DCR of 45.0%. In 14 NSCLC patients, 3 (21.4%) achieved PR and 5 (35.7%) achieved SD, with an ORR of 21.4% and DCR of 57.1%. The best objective responses (BOR) of the target lesions from the baseline and the duration of treatment for all patients are shown in FIGS. 23A and 23B. The median progression free survival (PFS), duration of response, and overall survival (OS) were not evaluated due to the small number of patents that were followed up and the relatively short duration of follow-up. For the 20 patients with no prior immunotherapy, the PR and SD rates were 40.0% (n=8) and 25.0% (n=5), respectively, with an ORR of 40.0% (n=8) and a DCR of 65.0% (n=13) (Table 26).

Of the 10 patients who had received prior anti-PD-1/PD-L1 therapy, two (20.0%) achieved PR, and one (10.0%) achieved SD, resulting in an ORR of 20.0% and a DCR of 30.0% (Table 26). FIG. 23C and FIG. 23D illustrates the percentage change from the baseline in shrinkage of the tumor in patients without any prior immunotherapy and those with prior anti-PD-1/PD-L1 therapy. A 55-year-old man patient with stage IV NPC, whose best tumor response was progressive disease (PD) on prior bispecific antibody targeting PD-L1/TGFβ as 3^{rd} line therapy, was enrolled in the 5 mg/kg group and achieved PR at week 7. The tumor CT scan for this patient is shown in FIG. 23E. A 46-year-old man patient with stage IV NSCLC, whose best tumor response was PR during prior nivolumab therapy (2^{nd} line) and SD during prior anti-4-1BB antibody therapy (4^{th} line), was enrolled in 10 mg/kg cohort, and achieved PR at week 13 (FIG. 23F).

### RP2D Determination

Based on the overall assessment of tolerability, PK, and pharmacodynamics, the regimen of 5 mg/kg Q3W was selected as RP2D for further investigation of PSB205 in advanced solid malignancies.

### Discussion

In accordance with the present invention, provided herein is the design and phase 1 clinical trial of PSB205, the first MabPair product with dual blockades of PD-1 and CTLA-4. PSB205 demonstrated encouraging anti-tumor response in a mixed cohorts of phase 1 study including patients have been previously treated with other PD-1/PD-L1 inhibitors. While the trial is still ongoing, the initial analysis also indicates a good overall safety profile with low incidence of grade 3 or higher TRAEs as compared other dual PD-1 and CTLA-4 blockades. The preliminary data supports further investigation of PSB205 for its potential in improving patient outcomes with increased anti-tumor response and tolerability for the treatment of solid malignancy. This study represents the first clinical testing of MabPair molecules. The strategy used to develop PSB205 may be applicable to other programs in which the optimal balance of efficacy and toxicity needs to be carefully maintained for each antibody component.

Manufactured to work as a single product, the two antibody components of PSB205 target and inhibit PD-1 and CTLA-4, respectively. In contrast to a bispecific antibody in which two arms of the antibody are locked in 1:1 ratio, MabPair product such as PSB205 enables its two antibody components to provide a distinct target-specific level of PK coverage and antibody effector function. This function can be achieved by adjusting the ratio in which the two antibodies are produced together in the CHO cell line and the PK profile of each antibody. The anti-PD-1 component of PSB205 is an IgG4 while the anti-CTLA-4 component is an IgG1 isotype. The Fc mediated effector mechanism of anti-CTLA-4 IgG1 may be critical for its effects on regulatory T cells in the tumor microenvironment. This is supported by the recent report of improved response to ipilimumab in patients with high affinity allele of Fc receptor (CD16a-V158)²⁰. In addition, CTLA-4 blockade can improve the priming of T cell response and increase the diversity of T cell clones, which may help bring new T cells to the tumor²⁵. However, prolonged T cell expansion can lead to immune related toxicity²⁶. How to adjust the level of CTLA-4 blockade to achieve the optimal balance of strong costimulation and T cell expansion by dual blockade of PD1 and CTLA4 pathways and local invigoration of tumor specific T cells by PD1 inhibition within each treatment cycle will be key to the success of combination therapy. The anti-CTLA-4 IgG1 of PSB205 was engineered to reduce binding to FcRn, which leads to a faster clearance in circulation. The elimination half-life of anti-CTLA4 antibody in PSB205 is about 5 days in humans, which is significantly shorter compared to the half-life of 15 days for ipilimumab. This allows more flexibility in controlling its exposure during dose titrations and quick elimination of the drug in the event of TRAEs. This unique feature may be translated into improved tumor response and better tolerability in humans.

PK analysis of the first in human study suggests we have achieved the design goal of bringing different level of target coverage for PD-1 and CTLA-4 after each dose of PSB205. The average t_{1/2} of aPD-1 and aCTLA-4 component after the first single administration of PSB205 in the 5 mg/kg dose group were about 8 and 4 days, respectively. The average t_{1/2} of aCTLA-4 after multiple administrations was about 5 days. aCTLA-4 in each dose group showed no obvious accumulation in the body after multiple administrations. In contrast, the average t_{1/2} of nivolumab and ipilimumab were about 19.1²⁷ and 15.4 days, respectively. Due to its long half-life, the level of ipilimumab can accumulate significantly after multiple treatment cycles when used every 3 weeks, which may contribute to the elevated irAEs. When nivolumab (3 mg/kg) and ipilimumab (1 mg/kg) were used together Q3W, significantly higher rate of irAEs was observed compared to the regimen in which ipilimumab (1 mg/kg) was dosed every 6 weeks²³. Lowering the dosing frequency of ipilimumab to every 6 weeks can reduce the stead state concentration of the antibody and improve the tolerability of the combination treatment. Because of its shorter half-life in aCTLA-4 component, it is possible to maintain the desire trough level of aCTLA-4 without significant accumulation after repeated administration of PSB205 when dosed every 3 weeks. This unique PK profile may contribute to the overall good safety profile of PSB205. Despite its relative faster clearance, there is strong evidence of functional blockade of CTLA-4 after PSB205 treatment. When dosed at 5 mg/kg, PSB205 can effectively induce the proliferation of CD8 cells and expansion of ICOS+CD4 T cells, indicating functional blockade of PD1 and CTLA4 pathways at the dose of 5 mg/kg, but would not lead to exacerbated irAEs.

In this study, PSB205 was generally well-tolerated, with grade 3 or higher TRAEs occurring in 7.1% of patients at RP2D. Previous studies have shown that the incidence of ≥grade 3 TRAEs in the combination of Opdivo and Yervoy ranges 22%-59%, with 32.8% in NSCLC¹⁷, 22% in MSI-H/dMMR Colorectal Cancer²⁸, 29%-53% in hepatocellular carcinoma²⁹, 30.3% in malignant pleural mesotheliom³⁰, 46% in renal cell carcinoma³¹ and 59% in melanoma³². Although the data are still maturing with the ongoing trial, the overall safety profile of PSB205 based on the preliminary data compares favorably to published data from other studies of anti-PD-1 and anti-CTLA-4 antibody combination. Fewer incidence of TRAEs will enable patients to stay with the treatment for longer period of time, which may contribute to the improved efficacy.

PSB205 also showed encouraging clinical activity in the present study. Ten (28.6%) out of 35 evaluable patients achieved partial responses, including those previously treated with other PD1/PDL1 inhibitors. Although the duration of the response needs to be established with a longer following up, initial observations of anti-tumor activity are promising, particularly for NPC, for which the overall response rate was 35% (7/20). Currently, there is no approved immune therapy for NPC yet. Several clinical trials for ≥2 line NPC immunotherapy are ongoing and the reported ORR ranges from 20.5% to 34% ³³⁻³⁶. NPC patients are shown to have elevated infiltration of Tregs in the tumor³⁷. They might be more sensitive to the combination of anti-PD-1 and anti-CTLA-4 antibodies. A recent phase 2 study of nivolumab (3 mg/kg, every 2 weeks) plus low-dose ipilimumab (1 mg/kg, every 6 weeks) in ≥2 line NPC patients reported an overall response rate of 30% (12/40); 86% (34/40) of patients experienced TRAEs, and 10% (4/40) experienced grade 3 or higher TRAEs ³⁸. The initial encouraging data observed in the present study indicate the potential of PSB205 in achieving an anti-tumor response similar to that obtained by the combination of nivolumab and low-dose ipilimumab, which warrants further clinical studies in NPC patients. The initial finding of anti-tumor response in PD-1 refractory patients with NSCLC and NPC suggest PSB205 can be explored in these hard to treat patient population, bringing the promise of combination therapy.

With its good safety profile and promising anti-tumor activity, PSB205 can be further developed as a backbone for additional combination studies with other therapeutic molecules such as small molecules, cancer vaccines, oncolytic viruses or therapeutic antibodies. Compared to conventional antibody combination therapy, which requires the administration of two drugs, MabPair products can be developed as single entities with a simple regulatory path. This will reduce the time and cost for developing antibody combination therapy.

In accordance with the present invention, a new approach is provided herein to deliver antibody combination therapy with a single vial product. PSB205 (QL1607), the first MabPair product with dual blockades of PD-1 and CTLA-4, has been evaluated in a phase 1 clinical trial and has shown an acceptable safety profile and early evidence of clinical anti-tumor activity in advanced solid malignancies.

### Materials and Methods

### The Design and Generation of PSB205

The development of the MabPair platform will be described in a separate report (Liu Z et al, manuscript in preparation). The generation and engineering of anti-human PD-1 antibody clone #1 and anti-human CTLA-4 antibody 11F4 were separately described (US2019/0248899 and US2019/0276542). The variable heavy (V_{H}) and variable light (V_{L}) genes of the anti-PD1 antibody were inserted into a human gamma-4 constant heavy chain and a constant kappa light chain, respectively. A substitution S228P at the hinge region was introduced to prevent the Fab arm exchange of IgG4. The variable heavy (V_{H}) and variable light (V_{L}) genes of the anti-CTLA-4 were inserted into a human gamma-1 constant heavy chain and a constant kappa light chain, respectively. several substitutions were introduced in anti-CTLA-4 IgG1 antibody to precisely control the cognate HC/HC and HC/LC chain pairings when co-expressed with anti-PD-1 IgG4 antibody in the same cells. Two substitutions (D399R and K409E) in C_{H}3 region of anti-CTLA-4 IgG1 were introduced to control the HC pairing. Three substitutions (K147D, F170C, and V173C) in C_{H}1 region, one substitution (C220G) in the upper hinge region of heavy chain and four substitutions (S131K, Q160C, S162C, and C214S) in Cκ region were introduced to control the correct pairing of LC in anti-CTLA-4 IgG1 antibody. In addition, one substitution (R255K) in C_{H}2 region was introduced to alter the binding of FcRn.

The MabPair cocktail was produced by multiple rounds of transient transfections in both Expi293 and ExpiCHO cells and purified with Protein A column. Mass spectrometry analysis confirmed that all HC/HC and HC/LC chains are correctly assembled without any mispairings.

### Production of PSB205 (QL1706) in a Stable CHO Cell Line

The DNAs encoding both HC and LC of anti-PD-1 IgG4 antibody are subcloned in pCHO1.0 vector (from Thermo Fisher) and used for the transfection and selection of CHO-S™ cell line. One stable cell line producing a high level of anti-PD-1 IgG4 antibody, clone G19G4-4B4, was selected as a host cell for introducing the LC and HC of engineered anti-CTLA-4 IgG1 antibody. Stable clones with high expression titer of both antibodies were further screened to identify a single clone of CHO cell that can produce anti-PD1 IgG4 and anti-CTLA-4 IgG1 antibodies at approximate 2:1 ratio.

### Clinical Trial

### Study Design and Patients

This was a phase I, open-label, dose escalation and expansion study to evaluate the safety, tolerability, MTD, PK and primary clinical activity of PSB205 in patients with advanced malignancy tumors.

First dose escalation was performed to determine DLT, MTD, and the RP2D of PSB205. The accelerated titration combined with the standard 3+3 dose escalation design was adopted. In brief, only one subject was enrolled in the first dose group. During the DLT evaluation period, if only drug-related AEs ≤Grade 2 were observed, subjects will be enrolled in the second dose group, which was performed using a standard 3+3 dose design. The maximum administered dose (MAD) is set at 10 mg/kg. In the process of dose escalation, subjects in the selected dose group were expanded, thus providing sufficient cases for the PK assessment of PSB205 in patients with advanced malignancies as well as the primary efficacy evaluation. The study protocol has been approved by the ethics committee of Sun Yat-sen University Cancer Center (No. A2019-091-1). All participants have provided written informed consent.

Patients who met the following key inclusion criteria were enrolled: (1) Male or female subjects aged 18 years or older; (2) pathologically confirmed diagnosis of advanced malignancies with failed standard treatment or no effective therapies, and for solid tumor, imaging measurable lesions were observed according to RECIST v1.1; (3) with Eastern Cooperative Oncology Group (ECOG) performance status of 0 or 1 and a life expectancy of greater than 3 months; (4) required functional levels of organs before the first drug administration; (5) agree to practice effective barrier contraception during the entire study treatment period and through 180 days after the last dose of study drug. The key exclusion criteria were: (1) previous or active autoimmune disease, interstitial lung disease and other diseases requiring long-term use of systemic corticosteroids (>10 mg/day prednisone or equivalent) or other immunosuppressive drugs; (2) grade 3 or 4 immune-related AEs related to prior cancer immunotherapy; (3) prior treatment with a CTLA-4 inhibitor in combination with a PD-1 or PD-L1 inhibitor; (4) female subjects who are pregnant or breastfeeding.

### Treatment

Five doses of PSB205 (0.3, 1, 3, 5, and 10 mg/kg) were administered every 3 weeks via intravenous infusion. Each subject received only one dose. Any Subject may be discontinued from the study for any of the following reasons: disease progressed (unless the investigators believed that there was a continuous clinical benefit), completed the study (up to 2 years), developed intolerable AEs, started a new anti-tumor treatment, or withdrew the informed consent, whichever comes first.

For the patient with solid tumors who had disease progression according to the RECIST v1.1 standard, PSB205 treatment will continue if the investigator judged that the subject was clinically stable and the benefits of continuing treatment were favored. At the same time, disease progression should be monitored by imaging examinations (interval ≥4 weeks). If disease progression was confirmed, but the investigator judged that the subject was clinically stable and the benefits of continuing treatment are favored, PSB205 treatment until no benefits.

### Outcomes

The primary outcomes were the safety and tolerability of PSB205, as defined by the incidence of AEs, SAES, and DLTs in patients with advanced malignant tumors, as well as the RP2D of PSB205. The secondary outcomes were PK, preliminary efficacy and immunogenicity of PSB205 in patients with advanced malignant tumors.

The correlation between PSB205 exposure and functional RO, and the correlation between biomarkers and PSB205 efficacy were also analyzed.

Safety analysis included all subjects receiving at least one dose of PSB205. The grading of AEs was done according to the Common Adverse Event Evaluation Criteria (CTCAE) v 5.0. In the dose-escalation phase, DLT was evaluated based on PSB205-related AEs occurring within 21 days (1 cycle) following the administration of the first dose of PSB205. According to the CTCAE v5.0 standard, DLT was defined as follows: Grade 3 or 4 non-hematological toxicity (except for Grade 3 fatigue, Grade 3 nausea/vomiting that resolves within 72 hours with appropriate supportive care), any Grade 4 hematological event (including Grade 4 thrombocytopenia), any Grade 3 thrombocytopenia with bleeding, or Grade 3 febrile neutropenia and any new steroid-use events (excluding subjects who are already on steroids). irAEs were mainly managed according to local medical practice. The investigator comprehensively evaluated the benefit/risk ratio of the subject and made a judgment on stopping/resuming dosing according to Management of Immune-Related Adverse Events in Patients Treated with Immune Checkpoint Inhibitor Therapy: American Society of Clinical Oncology Clinical Practice Guideline (2018 version)⁴².

For patients with solid tumors, tumor response was assessed according to RECIST v1.1. CT scans or Mills were performed at baseline, every 2 treatment cycles (6 weeks) in the first four cycles and every 3 treatment cycles (9 weeks) thereafter.

Immunogenicity assessments were performed using blood collected at pre-dose of each cycle.

### Pharmacokinetic Sampling Schedule and Assay Method

Plasma samples to characterize the pharmacokinetics of aCTLA-4 and aPD-1 were collected at the following timepoints. On cycle 1 day 1 to day 14 (single-dose) and cycle6 day 1 to day 14 (multiple-dose): at pre-dose, end of infusion, 2, 8, 24, 48, 72,168, 336 hours post-dose; on cycle 2 and other cycles: at predose, end of infusion and 48 h after end of infusion; end of visit and 30, 60 and 90 days after the last administration. These samples were analyzed using a validated enzyme-linked immunoadsordent assay (ELISA) method.

Pharmacokinetic analysis method

PK parameters of aCTLA-4 and aPD-1 were analyzed using the non-compartmental approach in the software WinNonlin version 8.2 (Certara USA, Inc., New Jersey, US). PK parameters for the single-dose stage included time to reach maximum concentration (Tₘₐₓ), maximum concentration (Cₘₐₓ), trough concentration (C_{trough}), area under the curve from time zero to the time of the last quantifiable concentration (AUC₀₋ₜ), percentage of the area under the curve derived after extrapolation (AUC__{%Extrap}), area under the curve from time zero to infinity (AUC_{0-inf}), area under the curve from time zero to day 21 (AUC_{0-21d}), elimination half-life (t_{1/2}), clearance (CL) and volume of distribution (V_{z}), and for the multiple-dose stage included Tₘₐₓ, Cₘₐₓ, C_{trough}, average concentration (C_{avg}), area under the curve over a dosing interval (AUC₀₋ₜₐᵤ, tau equals to 21 day), AUC_{0-inf}, AUC__{%Extrap}, t_{1/2}, clearance at steady state (CLₛₛ), volume of distribution at steady state (Vₛₛ), the accumulation ratio assessed by Cₘₐₓ (R_{ac_}Cₘₐₓ), the accumulation ratio assessed by C_{trough} (R_{ac_}C_{trough}), the accumulation ratio assessed by AUC (R_{ac_}AUC)ₒ

### Pharmacodynamic Assessments

Blood for PD-1 receptor occupancy detection is collected on cycle 1 at pre-dose, 72, 168 and 336 hours postdose, cycle 2 day 1 at pre-dose and 48 hours postdose as well as each imaging evaluation of tumor. Other biomarkers were detected in T cells in cycle 1 at pre-dose, 168 and 336 hours postdose, cycle 2 day 1 at pre-dose and 48 hours postdose as well as each imaging evaluation of tumor.

### Statistical Analysis

This phase I trial was designed to allow assessments of safety and tolerability based on an accelerated titration combined with the standard 3+3 dose-escalation design. The analyses of demographics, safety, and tolerability were descriptive. Estimate of ORR (including DCR) and its 95% CI were calculated by Exact Clopper-Pearson method based on evaluable patients. Comparisons between predose and postdose (Cycle1 168 h) values for certain pharmacodynamic markers were made using Wilcoxon Signed-Rank Test.

### TABLES AND TABLE LEGENDS

TABLE 17
Baseline characteristics of included patients

| | Patients | |
|---|---|---|
| Characteristic | (N = 47) | |
| Age (year) | | |
| Median (range) | 51.0 | (27-73) |
| Gender, n (%) | | |
| Male | 38 | (80.9) |
| Female | 9 | (19.1) |
| ECOG performance status, n (%) | | |
| 0 | 17 | (36.2) |
| 1 | 30 | (63.8) |
| Tumor type, n (%) | | |
| NSCLC | 20 | (42.6) |
| NPC | 25 | (53.2) |
| Thyroid cancer | 1 | (2.1) |
| Mucinous adenocarcinoma of the umbilical canal | 1 | (2.1) |
| Staging, n (%) | | |
| III | 1 | (2.1) |
| IV | 46 | (97.9) |
| History of immunotherapy, n (%) | | |
| None | 28 | (59.6) |
| aPD-1/aPD-L1^{[a]} | 13 | (27.7) |
| aPD-1/placebo | 5 | (10.6) |
| Other immunotherapy^{[a][b]} | 4 | (8.5) |
| No. of prior lines of therapy, n (%) | | |
| 0 | 1 | (2.1) |
| 1 | 18 | (38.3) |
| 2 | 13 | (27.7) |
| 3 | 7 | (14.9) |
| 4 | 4 | (8.5) |
| 5 | 3 | (6.4) |
| Unknown | 1 | (2.1) |
| Median (range) | 2.0 | (0-5) |

Abbreviations:
ECOG, Eastern Cooperative Oncology Group;
NSCLC, non-small-cell lung cancer;
NPC, nasopharyngeal carcinoma;
aPD-1, anti-programmed cell death protein 1;
aPD-L1, anti-PD-1 ligand;
^{[a]}One subject received bispecific antibody against PD-L1/TGFβ; One subject received two kinds of immunotherapy: anti-PD-1 antibody and anti-4-1BB antibody; One subject received two kinds of immunotherapy: anti-PD-1 antibody and anti-OX40 antibody; these 3 cases were recorded once both in “aPD-1/aPD-L1” and “other immunotherapy”.
^{[b]}Including drugs targeting OX40, 4-1BB and TGFβ.

TABLE 18
Treatment related adverse events occurring in ≥5% PSB205-treated patients

| | 1 mg/kg (N = 6) ^{a} | | 3 mg/kg (N = 6) ^{a} | | 5 mg/kg (N = 28) | | |
|---|---|---|---|---|---|---|---|
| | Grade 1 | Grade 2 | Grade 1 | Grade 2 | Grade 1 | Grade 2 | Grade ≥3 ^{c} |
| TRAEs, n(%) | 2 (33.3) | 3 (50.0) | 1 (16.7) | 1 (16.7) | 14 (50.0) | 3 (10.7) | 2 (7.1) |
| Pruritus | 2 (33.3) | 0 | 1 (16.7) | 1 (16.7) | 6 (21.4) | 0 | 0 |
| Rash | 2 (33.3) | 0 | 2 (33.3) | 0 | 4 (14.3) | 1 (3.6) | 0 |
| AST increased | 1 (16.7) | 0 | 0 | 0 | 5 (17.9) | 0 | 1 (3.6) |
| Fatigue | 1 (16.7) | 0 | 0 | 0 | 4 (14.3) | 0 | 0 |
| Hypothyroidism | 0 | 2 (33.3) | 1 (16.7) | 0 | 0 | 1 (3.6) | 0 |
| Hyperthyroidism | 0 | 1 (16.7) | 0 | 0 | 3 (10.7) | 0 | 0 |
| ALT increased | 0 | 1 (16.7) | 0 | 0 | 0 | 1 (3.6) | 0 |
| Pyrexia | 0 | 0 | 0 | 0 | 2 (7.1) | 0 | 0 |
| Infusion | 0 | 0 | 0 | 0 | 1 (3.6) | 1 (3.6) | 0 |
| related reaction | | | | | | | |

| | 10 mg/kg (N = 6) | | | | Total (N = 47) ^{b} | | | | | | | |
|---|---|---|---|---|---|---|---|---|---|---|---|---|
| | Grade 1 | Grade 2 | Grade ≥3^{d} | | Grade 1 | | Grade 2 | | Grade ≥3 | | Any Grade | |
| TRAEs, n(%) | 1 (16.7) | 1 (16.7) | 3 | (50.0) | 18 | (38.3) | 8 | (17.0) | 5 | (10.6) | 31 | (66.0) |
| Pruritus | 1 (16.7) | 0 | 0 | | 10 | (21.3) | 1 | (2.1) | 0 | | 11 | (23.4) |
| Rash | 0 | 1 (16.7) | 0 | | 8 | (17.0) | 2 | (4.3) | 0 | | 10 | (21.3) |
| AST increased | 0 | 0 | 0 | | 6 | (12.8) | 0 | | 1 | (2.1) | 7 | (14.9) |
| Fatigue | 0 | 1 (16.7) | 0 | | 5 | (10.6) | 1 | (2.1) | 0 | | 6 | (12.8) |
| Hypothyroidism | 0 | 1 (16.7) | 0 | | 1 | (2.1) | 4 | (8.5) | 0 | | 5 | (10.6) |
| Hyperthyroidism | 1 (16.7) | 0 | 0 | | 4 | (8.5) | 1 | (2.1) | 0 | | 5 | (10.6) |
| ALT increased | 2 (33.3) | 0 | 0 | | 2 | (4.3) | 2 | (4.3) | 0 | | 4 | (8.5) |
| Pyrexia | 1 (16.7) | 1 (16.7) | 0 | | 3 | (6.4) | 1 | (2.1) | 0 | | 4 | (8.5) |
| Infusion | 0 | 0 | 1 | (16.7)^{e} | 1 | (2.1) | 1 | (2.1) | 1 | (2.1) | 3 | (6.4) |
| related reaction | | | | | | | | | | | | |

Abbreviations:
TRAE, treatment related adverse event;
AST, aspartate aminotransferase;
ALT, alanine aminotransferase
^{a} No Grade ≥3 TRAE occurred in the dose level.
^{b} No TRAE occurred in the 0.3 mg/kg (N = 1)
^{c} One patient experienced myocarditis (grade 3).
^{d}One patient experienced platelet count decreased (grade 3) and immune-mediated nephritis (grade 4), one patient experienced platelet count decreased (grade 4).
^{e}Grade 4.

TABLE 19
Best objective response according to RECIST v1.1

| | 0.3 mg/kg | 1 mg/kg | 3 mg/kg | 5 mg/kg | 10 mg/kg | Total |
|---|---|---|---|---|---|---|
| All | (N = 1) | (N = 6) | (N = 6) | (N = 19) | (N = 3) | (N = 35) |
| BOR n(%) | | | | | | |
| CR | 0 | 0 | 0 | 0 | 0 | 0 |
| PR | 0 | 2 (33.3) | 1 (16.7) | 5 (26.3) | 2 (66.7) | 10 (28.6) |
| SD | 0 | 1 (16.7) | 2 (33.3) | 4 (21.1) | 0 | 7 (20.0) |
| PD | 1 (100) | 3 (50.0) | 3 (50.0) | 10 (52.6) | 1 (33.3) | 18 (51.4) |
| ORR, | 0 | 2 (33.3) | 1 (16.7) | 5 (26.3) | 2 (66.7) | 10 (28.6) |
| n (%) 95% CI | (0.000, | (4.327, | (0.421, | (9.147, | (9.430, | (14.635, |
| | 97.500) | 77.722) | 64.123) | 51.203) | 99.160) | 46.304) |
| DCR, | 0 | 3 (50.0) | 3 (50.0) | 9 (47.4) | 2 (66.7) | 17 (48.6) |
| n (%) 95% CI | (0.000, | (11.812, | (11.812, | (24.447, | (9.430, | (31.383, |
| | 97.500) | 88.188) | 88.188) | 71.136) | 99.160) | 66.011) |
| | 0.3 mg/kg | 1 mg/kg | 3 mg/kg | 5 mg/kg | 10 mg/kg | Total |
| NPC | (N = 0) | (N = 3) | (N = 3) | (N = 13) | (N = 1) | (N = 20) |
| BOR n(%) | | | | | | |
| CR | 0 | 0 | 0 | 0 | 0 | 0 |
| PR | 0 | 1 (33.3) | 0 | 5 (38.5) | 1 (100) | 7 (35.0) |
| SD | 0 | 0 | 1 (33.3) | 1 (7.7) | 0 | 2 (10.0) |
| PD | 0 | 2 (66.7) | 2 (66.7) | 7 (53.8) | 0 | 11 (55.0) |
| ORR, | 0 | 1 (33.3) | 0 | 5 (38.5) | 1 (100) | 7 (35.0) |
| n (%) 95% CI | (—, —) | (0.840, | (0.000, | (13.858, | (2.500, | (15.391, |
| | | 90.570) | 70.760) | 68.422) | 100.000) | 59.219) |
| DCR, | 0 | 1 (33.3) | 1 (33.3) | 6 (46.2) | 1 (100) | 9 (45.0) |
| n (%) 95% CI | (—, —) | (0.840, | (0.840, | (19.223, | (2.500, | (23.058, |
| | | 90.570) | 90.570) | 74.865) | 100.000) | 68.472) |
| | 0.3 mg/kg | 1 mg/kg | 3 mg/kg | 5 mg/kg | 10 mg /kg | Total |
| NSCLC | (N = 1) | (N = 3) | (N = 3) | (N = 5) | (N = 2) | (N = 14) |
| BORn(%) | | | | | | |
| CR | 0 | 0 | 0 | 0 | 0 | 0 |
| PR | 0 | 1 (33.3) | 1 (33.3) | 0 | 1 (50.0) | 3 (21.4) |
| SD | 0 | 1 (33.3) | 1 (33.3) | 3 (60.0) | 0 | 5 (35.7) |
| PD | 1 (100) | 1 (33.3) | 1 (33.3) | 2 (40.0) | 1 (50.0) | 6 (42.9) |
| ORR, | 0 | 1 (33.3) | 1 (33.3) | 0 | 1 (50.0) | 3 (21.4) |
| n (%) 95% CI | (0.000, | (0.840, | (0.840, | (0.000, | (1.258, | (4.658, |
| | 97.500) | 90.570) | 90.570) | 52.182) | 98.742) | 50.798) |
| DCR, | 0 | 2 (66.7) | 2 (66.7) | 3 (60.0) | 1 (50.0) | 8 (57.1) |
| n (%) 95% CI | (0.000, | (9.430, | (9.430, | (14.663, | (1.258, | (28.861, |
| | 97.500) | 99.160) | 99.160) | 94.726) | 98.742) | 82.339) |

Abbreviations: BOR, best overall response; CR, complete response; PR, partial response; SD, stable disease; PD, progressive disease; ORR, objective response rate; DCR, disease control rate; NPC, nasopharyngeal carcinoma; NSCLC, non-small-cell lung cancer.

TABLE 20
Summary of Preclinical Assessment of PSB103 (anti-PD1 IgG4)
Table 20: Summary of Preclinical Assessment of PSB103 (anti-PD1 IgG4)

| Antibody | PSB 103 | | Nivolumab analog | | Pembrolizumab analog | |
|---|---|---|---|---|---|---|
| Binding Affinity | 2.18E−09 | M | 1.4E−08 | M | 1.02E−08 | M |
| rHuman PD-1 | | | | | | |
| IC₅₀ PD-1 | 1.49 +/− 0.3 | nM | 6.03 +/− 0.85 | nM | 1.63 +/− 0.64 | nM |
| Reporter Assay | | | | | | |
| IC₅₀ Allo MLR * | 0.54 ± 0.07 | nM | 5.3 ± 1.1 | nM | 2.8 ± 2.2 | nM |
| IC₅₀ CMV Recall | 0.15 | nM | 0.52 | nM | 0.20 | nM |
| Response | | | | | | |
| PK t_{1/2} cyno | 297 ± 32 | hrs | 261 ± 226\hat{ } | hrs | 1. ± 38\hat{ } | hrs |

* IFNμ production readout for T cell activation
\hat{ }published report

TABLE 21
Summary of Preclinical Assessment of PSB105 (anti-CTLA-4 IgG1)

| Antibody | PSB 103 | | Nivolumab analog | | Pembrolizumab analog | |
|---|---|---|---|---|---|---|
| Binding Affinity | 2.18E−09 | M | 1.4E−08 | M | 1.02E−08 | M |
| rHuman PD-1 | | | | | | |
| IC₅₀ PD-1 | 1.49 +/− 0.3 | nM | 6.03 +/− 0.85 | nM | 1.63 +/− 0.64 | nM |
| Reporter Assay | | | | | | |
| IC₅₀ Allo MLR * | 0.54 ± 0.07 | nM | 5.3 ± 1.1 | nM | 2.8 ± 2.2 | nM |
| IC₅₀ CMV Recall | 0.15 | nM | 0.52 | nM | 0.20 | nM |
| Response | | | | | | |
| PK t_{1/2} cyno | 297 ± 32 | hrs | 261 ± 226\hat{ } | hrs | 142. ± 38\hat{ } | hrs |

| | anti-CTLA-4 | | |
|---|---|---|---|
| Antibody | mAb_10 | PSB105 | Ipilimumab |
| Binding to T cells (EC50 pM) | 85 | 158 | 126 |
| Competition CTLA-Fc | 8.3 | 16.8 | 17.4 |
| binding to Raji (IC50 pM) | | | |
| Serum half life in cyno (hr) | ND | 109 ± 9 | 397 ± 100 |

TABLE 22
Summary of anti-CTLA-4 IgG1 variants binding to human
FcRn/β 2M complex at pH 6.0 by Biacore analysis.

| Anti-CTLA4 | | Concentration | Rmax (RU) | Relative FcRn |
|---|---|---|---|---|
| clone | Mutation | (nM) | at pH 6.0 | Binding |
| 10D4 | Parent | 125 | 228 | 1.00 |
| | | 62.5 | 181 | 1.00 |
| 10D4 | M252A | 125 | 217 | 0.95 |
| | | 62.5 | 146 | 0.81 |
| 10D4 | R255K | 125 | 196 | 0.86 |
| | | 62.5 | 150 | 0.83 |
| 10D4 | M252L | 125 | 121 | 0.53 |
| | | 62.5 | 90.5 | 0.50 |
| 11F4 | Parent | 125 | 132 | 1.00 |
| (PSB105P) | | 62.5 | 85.1 | 1.00 |
| 11F4 | M252A | 125 | 101 | 0.77 |
| | | 62.5 | 73.5 | 0.86 |
| 11F4 | R255K | 125 | 101 | 0.77 |
| (PSB105) | | 62.5 | 73.9 | 0.87 |
| 11F4 | M252L | 125 | 54.1 | 0.41 |
| | | 62.5 | 38.2 | 0.45 |
| 1194 | Parent | 125 | 110 | 1.00 |
| | | 62.5 | 78.8 | 1.00 |
| 1194 | H435R | 125 | 78.8 | 0.72 |
| | | 62.5 | 52.6 | 0.67 |

TABLE 23
Sex-averaged Pharmacokinetic Parameters of Test Articles Following
Single i.v. Administration in Cynomolgus Monkeys

| Test | | t_{1/2} | Tₘₐₓ | Cₘₐₓ | AUC₀₋ₗₐₛₜ | AUC_{0-∞} | Vz | Cl |
|---|---|---|---|---|---|---|---|---|
| Article | Parameter | (hr) | (hr) | (μg/mL) | (hr*μg/mL | (hr*μg/mL) | (mL/kg) | (mL/hr/kg) |
| PSB103 | N^{a} | 4 | 4 | 4 | 4 | 4 | 4 | 4 |
| (29102) | Mean | 297 | 2.17^{b} | 204 | 37300 | 48800 | 43.9 | 0.106 |
| | SD | 64.8 | 3.89 | 23.5 | 3540 | 10800 | 1.05 | 0.0203 |
| Human | N^{a} | 4 | 4 | 4 | 4 | 4 | 4 | 4 |
| IgG4 | Mean | 181 | 2.06^{b} | 138 | 16900 | 21000 | 64.5 | 0.270 |
| (16102) | SD | 51.0 | 3.96 | 19.9 | 8060 | 8420 | 9.27 | 0.108 |
| PSB105 | N | 2 | 2 | 2 | 2 | 2 | 2 | 2 |
| (10511) | Mean | 109 | 0.290 | 116 | 6080 | 6140 | 76.6 | 0.489 |
| | SD | 1.66 | 0.297 | 26.1 | 283 | 287 | 4.75 | 0.0229 |
| PSB105P | N | 2 | 2 | 2 | 2 | 2 | 2 | 2 |
| (10511P) | Mean | 125 | 0.08 | 153 | 10100 | 10300 | 52.3 | 0.291 |
| | SD | 21.2 | 0.00 | 4.67 | 440 | 631 | 5.69 | 0.0177 |
| ipilimumab | N | 2 | 2 | 2 | 2 | 2 | 2 | 2 |
| | Mean | 397 | 0.08 | 128 | 22500 | 31900 | 53.5 | 0.0948 |
| | SD | 104 | 0.00 | 4.67 | 93.1 | 3740 | 7.80 | 0.0111 |

IgG1 = Immunoglobulin G1;
SD = standard deviation

TABLE 24
Pharmacokinetic Parameters of aCTLA-4 and aPD-1 after Intravenous Infusion of PSB205 (QL1706)

| PK | Cycle 1 (first-dose) | | | | |
|---|---|---|---|---|---|
| Parameters | 0.3 mg/kg | 1 mg/kg | 3 mg/kg | 5 mg/kg | 10 mg/kg |
| (unit) | (n = 1) | (n = 6) | (n = 6) | (n = 17) | (n = 6) |
| aCTLA-4 | | | | | |
| Cₘₐₓ | 2.27 | 7.86 | 24.1 | 38.5 | 73.6 |
| (μg/mL) | [1] | (11%)[6] | (11%)[6] | (16%)[17] | (19%)[6] |
| Tₘₐₓ | 2.50 | 2.50 | 0.54 | 0.55 | 0.63 |
| (h) | [1] | (0.50, 48.40)[6] | (0.52, 2.50)[6] | (0.50, 2.58)[17] | (0.52, 2.53)[6] |
| C_{avg} | — | — | — | — | — |
| (μg/mL) | | | | | |
| C_{trough} | — | — | — | — | — |
| (μg/mL) | | | | | |
| AUC₀₋ₜ | 1.22 | 1170 | 3010 | 4330 | 8780 |
| (μg*h/mL) | [1] | (27%)[6] | (33%)[6] | (36%)[13]^{a} | (56%)[3] [symbol] |
| AUC_{0-21 d} | — | 1190 | 3020 | 4390 | 8300 |
| (μg*h/mL) | | (25%)[6] | (32%)[6] | (35%)[13] [symbol] | (41%)[6]] |
| AUC_{0-∞} | — | 1270 | 3180 | 4600 | 9020 |
| (μg*h/mL) | | (26%)[6] | (34%)[6] | (38%)[13] [symbol] | (51%)[6] |
| AUC_{_{—}}_{% Extrap} | — | 8 | 5 | 5 | 10 |
| (%) | | (26%)[6] | (24%)[6] | (68%)[13] [symbol] | (77%)[6] |
| t_{1/2} | — | 121 | 116 | 104 | 119 |
| (h) | | (8%)[6] | (13%)[6] | (27%)[13]^{b} | (46%)[6] |
| CL | — | 0.0159 | 0.0195 | 0.0238 | 0.0252 |
| (L/h) | | (36%)[6] | (38%)[6] | (35%)[13] [symbol] | (28%)[6] |
| V_{z} | — | 2.72 | 3.24 | 3.37 | 3.92 |
| (L) | | (28%)[6] | (39%)[6] | (28%)[13]^{b} | (16%)[6] |
| R [symbol] Cₘₐₓ | — | — | — | — | — |
| R [symbol] AUC | — | — | — | — | — |
| R [symbol] C_{trough} | — | — | — | — | — |
| aPD-1 | | | | | |
| Cₘₐₓ | 44.25 | 13.8 | 49.5 | 76.5 | 157 |
| (μg/mL) | [1] | (19%)[6] | (12%)[6] | (21%)[17] | (15%)[6] |
| Tₘₐₓ | 2.50 | 0.52 | 0.53 | 0.57 | 1.58 |
| (h) | [1] | (0.50, 24.62)[6] | (0.52, 5.7)[6] | (0.50, 166.72)[17] | (0.52, 8.50)[6] |
| C_{avg} | — | — | — | — | — |
| (μg/mL) | | | | | |
| C_{trough} | 0.529 | 2.80 | 10.4 | 11.6 | 21.5 |
| (μg/mL) | [1] | (29%)[6] | (22%)[6] | (47%)[03] [symbol] | (8.8%)[3]^{a} |
| AUC₀₋ₜ | 643 | 3160 | 9610 | 14800 | 27900 |
| (μg*h/mL) | [1] | (17%)[6] | (21%)[6] | (27%)[13] [symbol] | (19%)[3] [symbol] |
| AUC_{0-21 d} | 643 | 3080 | 11000 | 14100 | 25100 |
| (μg*h/mL) | [1] | (20%)[5] [symbol] | (12%)[3] [symbol] | (24%)[11] [symbol] | (23500, 26700)[2]^{c} |
| AUC_{0-∞} | — | 3060 | — | 15600 | 27300 |
| (μg*h/mL) | | [1]^{b} | | (36%)[7]^{b} | (26600, 28000)[2]^{b} |
| AUC_{_{—}}_{% Extrap} | — | 17 | — | 14 | 9 |
| (%) | | [1]^{b} | | (32%)[7]^{b} | (5, 13)[2]^{b} |
| t_{1/2} | — | 227 | — | 187 | 147 |
| (h) | | [1]^{b} | | (33%)[7]^{b} | (128, 167)[2] [symbol] |
| CL | — | 0.0122 | — | 0.0145 | 0.0159 |
| (L/h) | | [1]^{b} | | (17%)[7]^{b} | (0.0146, 0.0172)[2]^{b} |
| V_{z} | — | 3.98 | — | 3.77 | 3.41 |
| (L) | | [1]^{b} | | (22%)[7]^{b} | (2.70, 4.13)[2]^{b} |
| R [symbol] Cₘₐₓ | — | — | — | — | — |
| R [symbol] AUC | — | — | — | — | — |
| R [symbol] C_{trough} | — | — | — | — | — |

| PK | Cycle 6 (Multiple-dose) | | |
|---|---|---|---|
| Parameters | 1 mg/kg | 3 mg/kg | 5 mg/kg |
| (unit) | (n = 3) | (n = 2) | (n = 4) |
| aCTLA-4 | | | |
| Cₘₐₓ | 8.06 | 23.6 | 38.1 |
| (μg/mL) | (16%)[3] | (23.3, 23.9)[2] | (17%)[4] |
| Tₘₐₓ | 0.52 | 13.53 | 0.53 |
| (h) | (0.52, 0.55)[3] | (2.50, 24.55)[2] | (0.53, 2.50)[4] |
| C_{avg} | 2.63 | 5.86 | 10.2 |
| (μg/mL) | (5%)[3] | (5.45, 6.27)[2] | (34%)[3]^{f} |
| C_{trough} | 0.749 | 0.761 | 2.22 |
| (μg/mL) | (33%)[3] | (0.536, 0.985)[2] | (59%)[3]^{d} |
| AUC₀₋ₜ | 1350 | 2960 | 5070 |
| (μg*h/mL) | (3%)[3] | (2750, 3160)[21] | (34%)[3]^{d} |
| AUC_{0-21 d} | 1320 | 2960 | 5120 |
| (μg*h/mL) | (5%)[3] | (2750, 3160)[2] | (34%)[3]^{f} |
| AUC_{0-∞} | 1560 | 3080 | 5490 |
| (μg*h/mL) | (6%)[3] | (2820, 3340)[2] | (36%)[3] [symbol] |
| AUC_{_{—}}_{% Extrap} | 13 | 4 | 7 |
| (%) | (30%)[ 3] | (3, 5)[2] | (55%)[3] [symbol] |
| t_{1/2} | 190 | 111 | 121 |
| (h) | (2%)[3] | (94.9, 127)[2] | (24%)[3] [symbol] |
| CL | 0.0134 | 0.0180 | 0.0225 |
| (L/h) | (27%)[3] | (0.0172, 0.0187)[2] | (53%)[3] [symbol] |
| V_{z} | 3.50 | 2.65 | 3.71 |
| (L) | (24%)[3] | (2.60, 2.69)[2] | (38%)[3]^{g} |
| R [symbol] Cₘₐₓ | 1.04 | 0.888 | 0.862 |
| | (6%)[3] | (0.866, 0.911)[2] | (10%)[4] |
| R [symbol] AUC | 0.977 | 1.05 | 1.03 |
| | (15%)[3] | (0.992, 1.12)[2] | (6%)[3]^{h} |
| R [symbol] C_{trough} | 1.33 | 0.922 | 1.11 |
| | (43%)[3] | (0.752, 1.09)[2] | (0.777, 1.44)[2]ⁱ |
| aPD-1 | | | |
| Cₘₐₓ | 21.2 | 66.6 | 110 |
| (μg/mL) | (12%)[3] | (66.5, 66.7)[2] | (27%)[4] |
| Tₘₐₓ | 2.50 | 0.53 | 5.57 |
| (h) | (0.52, 2.50)[3] | (0.52, 0.53)[2] | (0.53, 8.67)[4] |
| C_{avg} | 11.0 | 34.9 | — |
| (μg/mL) | (13%)[3] | (34.6, 35.2)[2] | |
| C_{trough} | 6.57 | 20.1 | 37.1 |
| (μg/mL) | (28%)[3] | (19.6, 20.5)[2] | (54%)[3]^{d} |
| AUC₀₋ₜ | 5780 | 17600 | 27800 |
| (μg*h/mL) | (6%)[3] | (17400, 17700)[2] | (42%)[3] [symbol] |
| AUC_{0-21 d} | 5540 | 17600 | — |
| (μg*h/mL) | (13%) [3] | (17400, 17700)[2] | |
| AUC_{0-∞} | — | — | — |
| (μg*h/mL) | | | |
| AUC_{_{—}}_{% Extrap} | — | — | — |
| (%) | | | |
| t_{1/2} | — | — | — |
| (h) | | | |
| CL | 0.00720 | 0.00676 | — |
| (L/h) | (21%)[3] | (0.00662, 0.00690)[2] | |
| V_{z} | — | — | — |
| (L) | | | |
| R [symbol] Cₘₐₓ | 1.64 | 1.24 | 1.36 |
| | (30%)[3] | (1.16, 1.31)[2] | (10%)[4] |
| R [symbol] AUC | 1.84 | 1.52 | — |
| | (34%)[3] | [1]^{h} | |
| R [symbol] C_{trough} | 2.22 | 1.79 | 2.45 |
| | (46%)[3] | (1.68, 1.90)[2] | (25%)[3]ⁱ |

Note 1:
Pharmacokinetic parameters are presented as mean ± SD (CV %)[N] where N is the number of data included in the statistical description. If N is 2 then all parameters are presented as median (minimum, maximum) [2]. Tₘₐₓ and Tₗₐₛₜ are presented as median (minimum, maximum) [N].
Note 2:
The results of AUC_{0-21 d}, V_{z} and CL for Cycle 6 were reported using the results of AUC₀₋ₜₐᵤ, Vₛₛ and CLₛₛ in Cycle 6, respectively.
^{a}In Cycle 1, subject 01040, 01041, 01042 and 01048 in 5 mg/kg cohort and subject 01030, 01036 and 01037 in 10 mg/kg cohort did not collect all the samples, thus, C_{trough} and AUC₀₋ₜ of those subjects were not included in the analysis.
^{b}In Cycle1, λ_{z} was not estimated accurately when AUC_{_{—}}_{% Extrap} >20% and R²_adjust <0.9, thus, parameters for some subjects that were calculated based on λ_{z} including AUC_{0-∞}, AUC_{_{—}}_{% Extrap}, CL, V_{z}, t_{1/2} were not summarized in the analysis if λ_{z} was inaccurate.
^{c}In Cycle1, AUC_{0-21 d} for some subjects were not included in the analysis when λ_{z} was not estimated accurately, but on this condition, if Tₗₐₛₜ < tau (504 h) and the difference between Tₗₐₛₜ and tau was no more than 1%, the results of AUC_{0-21 d} were replaced with AUC₀₋ₜ values and were included in the analysis.
^{d}In Cycle 6, subject 01018 in 5 mg/kg cohort did not collect all the samples, thus, C_{trough} and AUC₀₋ₜ of this subject were not included in the analysis.
e. In Cycle 6, λ_{z} was not estimated accurately when AUC_{_{—}}_{% Extrap} >20% and R²_adjust <0.9, thus, parameters for some subjects that were calculated based on λ_{z} including AUC_{0-∞}, AUC_{_{—}}_{% Extrap}, t_{1/2} were not summarized in the analysis if λ_{z} was inaccurate.
^{f}In Cycle 6, when AUC₀₋ₜₐᵤ was not estimated accurately, parameters that were calculated based on AUC₀₋ₜₐᵤ such as C_{avg}, CLₛₛ were not summarized in the analysis.
^{g}In Cycle 6, if either λ_{z} or AUC₀₋ₜₐᵤ was not accurately estimated, Vₛₛ which was based on the two was not summarized.
^{h}In Cycle 6, if either AUC_{0-21 d} in Cycle 1 or AUC₀₋ₜₐᵤ in Cycle 6 for any subject were not accurately estimated then R_{ac —}AUC of those were considered inaccurate and were not summarized.
ⁱIn Cycle 6, if subjects did not collect all samples either in Cycle 1 or in Cycle 6 then R_{ac —}C_{trough} of those were considered inaccurate and were not summarized. [symbol]
indicates data missing or illegible when filed

TABLE 25
Immune-related AE

| | 1 mg/kg | | 3 mg/kg | | 5 mg/kg | | | 10 mg/kg |
|---|---|---|---|---|---|---|---|---|
| | (N = 6) ^{a} | | (N = 6) ^{a} | | (N = 28) | | | (N = 6) |
| | Grade 1 | Grade 2 | Grade 1 | Grade 2 | Grade 1 | Grade 2 | Grade ≥3 | Grade 1 |
| irAEs, n(%) | 1 (16.7) | 2 (33.3) | 1 (16.7) | 1 (16.7) | 6 (21.4) | 2 (7.1) | 1 (3.6) | 0 |
| Pruritus | 2 (33.3) | 0 | 1 (16.7) | 1 (16.7) | 6 (21.4) | 0 | 0 | 1 (16.7) |
| Rash | 2 (33.3) | 0 | 2 (33.3) | 0 | 4 (14.3) | 1 (3.6) | 0 | 0 |
| Hypothyroidism | 0 | 2 (33.3) | 1 (16.7) | 0 | 0 | 1 (3.6) | 0 | 0 |
| Hyperthyroidism | 0 | 1 (16.7) | 0 | 0 | 3 (10.7) | 0 | 0 | 1 (16.7) |
| Immune- | 0 | 0 | 0 | 0 | 0 | 0 | 0 | 0 |
| mediated | | | | | | | | |
| nephritis | | | | | | | | |
| Dermatitis | 0 | 0 | 0 | 0 | 1 (3.6) | 0 | 0 | 0 |
| Myocarditis | 0 | 0 | 0 | 0 | 0 | 0 | 1 (3.6) | 0 |
| Blood thyroid | 0 | 0 | 1 (16.7) | 0 | 0 | 0 | 0 | 0 |
| stimulating | | | | | | | | |
| hormone | | | | | | | | |
| decreased | | | | | | | | |

| | | | Total | | | |
|---|---|---|---|---|---|---|
| | 10 mg/kg | | (N = 47) ^{b} | | | |
| | (N = 6) | | | | | Any |
| | Grade 2 | Grade ≥3 | Grade 1 | Grade 2 | Grade ≥3 | Grade |
| irAEs, n(%) | 1 (16.7) | 1 (16.7) | 8 (17.0) | 6 (12.8) | 2 (4.3) | 16 (34.0) |
| Pruritus | 0 | 0 | 10 (21.3) | 1 (2.1) | 0 | 11 (23.4) |
| Rash | 1 (16.7) | 0 | 8 (17.0) | 2 (4.3) | 0 | 10 (21.3) |
| Hypothyroidism | 1 (16.7) | 0 | 1 (2.1) | 4 (8.5) | 0 | 5 (10.6) |
| Hyperthyroidism | 0 | 0 | 4 (8.5) | 1 (2.1) | 0 | 5 (10.6) |
| Immune- | 0 | 1 (16.7) | 0 | 0 | 1 (2.1) | 1 (2.1) |
| mediated | | | | | | |
| nephritis | | | | | | |
| Dermatitis | 0 | 0 | 1 (2.1) | 0 | 0 | 1 (2.1) |
| Myocarditis | 0 | 0 | 0 | 0 | 1 (2.1) | 1 (2.1) |
| Blood thyroid | 0 | 0 | 1 (2.1) | 0 | 0 | 1 (2.1) |
| stimulating | | | | | | |
| hormone | | | | | | |
| decreased | | | | | | |

Abbreviations: irAE, immune-related adverse event
^{a} No Grade ≥3 irAE occurred in the dose level.
^{b} There was no irAE occurred in the 0.3 mg/kg (N = 1).

TABLE 26
Best objective response based on prior immunotherapy history

| | Naïve to prior | Received prior anti- | Received prior | |
|---|---|---|---|---|
| | immunotherapy | PD-1/PD-L1 therapy | anti-PD-1/Placebo | Total |
| All | (N = 20) | (N = 10) | (N = 5) | (N = 35) |
| BOR n(%) | | | | |
| CR | 0 | 0 | 0 | 0 |
| PR | 8 (40.0) | 2 (20.0) | 0 | 10 (28.6) |
| SD | 5 (25.0) | 1 (10.0) | 1 (20.0) | 7 (20.0) |
| PD | 7 (35.0) | 7 (70.0) | 4 (80.0) | 18 (51.4) |
| ORR, | 8 (40.0) | 2 (20.0) | 0 | 10 (28.6) |
| n (%) 95% CI | (19.119, 63.946) | (2.521, 55.610) | (0.000, 52.182) | (14.635, 46.304) |
| DCR, | 13 (65.0) | 3 (30.0) | 1 (20.0) | 17 (48.6) |
| n (%) 95% CI | (40.781, 84.609) | (6.674, 65.245) | (0.505, 71.642) | (31.383, 66.011) |

Abbreviations:
BOR, best overall response;
CR, complete response;
PR, partial response;
SD, stable disease;
PD, progressive disease;
ORR, objective response rate;
DCR, disease control rate

### REFERENCES

1. Swain S M, Baselga J, Kim S-B, Ro J, Semiglazov V, Campone M, et al. Pertuzumab, Trastuzumab, and Docetaxel in HER2-Positive Metastatic Breast Cancer. New England Journal of Medicine. 2015; 372(8):724-734.
2. Finn R S, Qin S, Ikeda M, Galle P R, Ducreux M, Kim T Y, et al. Atezolizumab plus Bevacizumab in Unresectable Hepatocellular Carcinoma. The New England journal of medicine. 2020; 382(20):1894-1905.
3. Brunet J F, Denizot F, Luciani M F, Roux-Dosseto M, Suzan M, Maffei M G, et al. A new member of the immunoglobulin superfamily—CTLA-4. Nature. 1987; 328(6127):267-270.
4. Ishida Y, Agata Y, Shibahara K, Honjo T. Induced expression of PD-1, a novel member of the immunoglobulin gene superfamily, upon programmed cell death. EMBO J. 1992; 11(11):3887-3895.
5. Fife B T, Bluestone J A. Control of peripheral T-cell tolerance and autoimmunity via the CTLA-4 and PD-1 pathways. Immunol Rev. 2008; 224:166-182.
6. Wing K, Onishi Y, Prieto-Martin P, Yamaguchi T, Miyara M, Fehervari Z, et al. CTLA-4 control over Foxp3+ regulatory T cell function. Science. 2008; 322(5899):271-275.
7. Pardoll DM. The blockade of immune checkpoints in cancer immunotherapy. Nat Rev Cancer. 2012; 12(4):252-264.
8. Chen L, Flies DB. Molecular mechanisms of T cell co-stimulation and co-inhibition. Nat Rev Immunol. 2013; 13(4):227-242.
9. Robert C, Long GV, Brady B, Dutriaux C, Maio M, Mortier L, et al. Nivolumab in previously untreated melanoma without BRAF mutation. The New England journal of medicine. 2015; 372(4):320-330.
10. Wolchok J D, Hodi F S, Weber J S, Allison J P, Urba W J, Robert C, et al. Development of ipilimumab: a novel immunotherapeutic approach for the treatment of advanced melanoma. Ann N Y Acad Sci. 2013; 1291:1-13.
11. Wei S C, Levine J H, Cogdill A P, Zhao Y, Anang N A S, Andrews M C, et al. Distinct Cellular Mechanisms Underlie Anti-CTLA-4 and Anti-PD-1 Checkpoint Blockade. Cell. 2017; 170(6):1120-1133 e1117.
12. Wei S C, Anang NAS, Sharma R, Andrews MC, Reuben A, Levine J H, et al. Combination anti-CTLA-4 plus anti-PD-1 checkpoint blockade utilizes cellular mechanisms partially distinct from monotherapies. Proc Natl Acad Sci USA. 2019; 116(45):22699-22709.
13. Wei S C, Duffy CR, Allison JP. Fundamental Mechanisms of Immune Checkpoint Blockade Therapy. Cancer Discov. 2018; 8(9):1069-1086.
14. Rotte A. Combination of CTLA-4 and PD-1 blockers for treatment of cancer. Journal of experimental & clinical cancer research: CR. 2019; 38(1):255.
15. Kooshkaki O, Derakhshani A, Hosseinkhani N, Torabi M, Safaei S, Brunetti O, et al. Combination of Ipilimumab and Nivolumab in Cancers: From Clinical Practice to Ongoing Clinical Trials. International journal of molecular sciences. 2020; 21(12).
16. Larkin J, Chiarion-Sileni V, Gonzalez R, Grob J J, Rutkowski P, Lao C D, et al. Five-Year Survival with Combined Nivolumab and Ipilimumab in Advanced Melanoma. The New England journal of medicine. 2019; 381(16):1535-1546.
17. Hellmann M D, Paz-Ares L, Bernabe Caro R, Zurawski B, Kim S W, Carcereny Costa E, et al. Nivolumab plus Ipilimumab in Advanced Non-Small-Cell Lung Cancer. The New England journal of medicine. 2019; 381(21):2020-2031.
18. Rizvi N A, Cho B C, Reinmuth N, Lee K H, Luft A, Ahn M J, et al. Durvalumab With or Without Tremelimumab vs Standard Chemotherapy in First-line Treatment of Metastatic Non-Small Cell Lung Cancer: The MYSTIC Phase 3 Randomized Clinical Trial. JAMA oncology. 2020; 6(5):661-674.
19. Selby M J, Engelhardt J J, Quigley M, Henning K A, Chen T, Srinivasan M, et al. Anti-CTLA-4 antibodies of IgG2a isotype enhance antitumor activity through reduction of intratumoral regulatory T cells. Cancer Immunol Res. 2013; 1(1):32-42.
20. Arce Vargas F, Furness AJS, Litchfield K, Joshi K, Rosenthal R, Ghorani E, et al. Fc Effector Function Contributes to the Activity of Human Anti-CTLA-4 Antibodies. Cancer Cell. 2018; 33(4):649-663 e644.
21. Simpson TR, Li F, Montalvo-Ortiz W, Sepulveda M A, Bergerhoff K, Arce F, et al. Fc-dependent depletion of tumor-infiltrating regulatory T cells co-defines the efficacy of anti-CTLA-4 therapy against melanoma. J Exp Med. 2013; 210(9):1695-1710.
22. Sznol M, Ferrucci P F, Hogg D, Atkins M B, Wolter P, Guidoboni M, et al. Pooled Analysis Safety Profile of Nivolumab and Ipilimumab Combination Therapy in Patients With Advanced Melanoma. Journal of clinical oncology: official journal of the American Society of Clinical Oncology. 2017; 35(34):3815-3822.
23. Hellmann M D, Rizvi N A, Goldman J W, Gettinger S N, Borghaei H, Brahmer J R, et al. Nivolumab plus ipilimumab as first-line treatment for advanced non-small-cell lung cancer (CheckMate 012): results of an open-label, phase 1, multicohort study. The Lancet Oncology. 2017; 18(1):31-41.
24. Perets R, Bar J, Rasco D W, Ahn M J, Yoh K, Kim D W, et al. Safety and efficacy of quavonlimab, a novel anti-CTLA-4 antibody (MK-1308), in combination with pembrolizumab in first-line advanced non-small-cell lung cancer. Annals of oncology: official journal of the European Society for Medical Oncology. 2020.
25. Kvistborg P, Philips D, Kelderman S, Hageman L, Ottensmeier C, Joseph-Pietras D, et al. Anti-CTLA-4 therapy broadens the melanoma-reactive CD8+ T cell response. Science translational medicine. 2014; 6(254):254ra128.
26. Oh DY, Cham J, Zhang L, Fong G, Kwek S S, Klinger M, et al. Immune Toxicities Elicted by CTLA-4 Blockade in Cancer Patients Are Associated with Early Diversification of the T-cell Repertoire. Cancer Res. 2017; 77(6):1322-1330.
27. Lee KW, Lee DH, Kang JH, Park JO, Kim SH, Hong Y S, et al. Phase I Pharmacokinetic Study of Nivolumab in Korean Patients with Advanced Solid Tumors. The oncologist. 2018; 23(2):155-e117.
28. Lenz H-J, Lonardi S, Zagonel V, Van Cutsem E, Limon ML, Wong K Y M, et al. Nivolumab plus low-dose ipilimumab as first-line therapy in microsatellite instability-high/DNA mismatch repair deficient metastatic colorectal cancer: Clinical update. Journal of Clinical Oncology. 2020; 38(4_suppl):11-11.
29. Yau T, Kang YK, Kim TY, El-Khoueiry A B, Santoro A, Sangro B, et al. Efficacy and Safety of Nivolumab Plus Ipilimumab in Patients With Advanced Hepatocellular Carcinoma Previously Treated With Sorafenib: The CheckMate 040 Randomized Clinical Trial. JAMA oncology. 2020; 6(11).
30. Baas P, Scherpereel A, Nowak A, Fujimoto N, Zalcman GJJoTO. ID:2908 First-Line Nivolumab+Ipilimumab vs Chemotherapy in Unresectable Malignant Pleural Mesothelioma: CheckMate 743. 2020; 15(10):e42.
31. Motzer R J, Tannir N M, McDermott D F, Aran Frontera O, Melichar B, Choueiri T K, et al. Nivolumab plus Ipilimumab versus Sunitinib in Advanced Renal-Cell Carcinoma. The New England journal of medicine. 2018; 378(14):1277-1290.
32. Hodi F S, Chiarion-Sileni V, Gonzalez R, Grob J J, Rutkowski P, Cowey C L, et al. Nivolumab plus ipilimumab or nivolumab alone versus ipilimumab alone in advanced melanoma (CheckMate 067): 4-year outcomes of a multicentre, randomised, phase 3 trial. The Lancet Oncology. 2018; 19(11):1480-1492.
33. Fang W, Yang Y, Ma Y, Hong S, Lin L, He X, et al. Camrelizumab (SHR-1210) alone or in combination with gemcitabine plus cisplatin for nasopharyngeal carcinoma: results from two single-arm, phase 1 trials. The Lancet Oncology. 2018; 19(10):1338-1350.
34. Hsu C, Lee SH, Ejadi S, Even C, Cohen RB, Le Tourneau C, et al. Safety and Antitumor Activity of Pembrolizumab in Patients With Programmed Death-Ligand 1-Positive Nasopharyngeal Carcinoma: Results of the KEYNOTE-028 Study. Journal of clinical oncology: official journal of the American Society of Clinical Oncology. 2017; 35(36):4050-4056.
35. Ma B B Y, Lim W T, Goh B C, Hui E P, Lo K W, Pettinger A, et al. Antitumor Activity of Nivolumab in Recurrent and Metastatic Nasopharyngeal Carcinoma: An International, Multicenter Study of the Mayo Clinic Phase 2 Consortium (NCI-9742). Journal of clinical oncology: official journal of the American Society of Clinical Oncology. 2018; 36(14):1412-1418.
36. Wang FH, Wei XL, Feng J, Li Q, Xu N, Hu X C, et al. Efficacy, Safety, and Correlative Biomarkers of Toripalimab in Previously Treated Recurrent or Metastatic Nasopharyngeal Carcinoma: A Phase II Clinical Trial (POLARIS-02). Journal of clinical oncology: official journal of the American Society of Clinical Oncology. 2021:Jco2002712.
37. Lau K M, Cheng S H, Lo K W, Lee S A, Woo J K, van Hasselt C A, et al. Increase in circulating Foxp3+CD4+CD25(high) regulatory T cells in nasopharyngeal carcinoma patients. British journal of cancer. 2007; 96(4):617-622.
38. Kao H, Ang M, Ng QS, Tan D S W, Tan W, Rajasekaran T, et al. 266O—Combination ipilimumab and nivolumab in recurrent/metastatic nasopharyngeal carcinoma (RIM NPC): Updated efficacy and safety analysis of NCT03097939. Annals of Oncology. 2020; 31(suppl_6): S1347-51354.
39. Socinski M A, Jotte R M, Cappuzzo F, Orlandi F, Stroyakovskiy D, Nogami N, et al. Atezolizumab for First-Line Treatment of Metastatic Nonsquamous NSCLC. The New England journal of medicine. 2018; 378(24):2288-2301.
40. McGregor B A, McKay R R, Braun DA, Werner L, Gray K, Flaifel A, et al. Results of a Multicenter Phase II Study of Atezolizumab and Bevacizumab for Patients With Metastatic Renal Cell Carcinoma With Variant Histology and/or Sarcomatoid Features. Journal of clinical oncology: official journal of the American Society of Clinical Oncology. 2020; 38(1):63-70.
41. Kudo M, Matilla A, Santoro A, Melero I, Gracian AC, Acosta-Rivera M, et al. Checkmate-040: Nivolumab (NIVO) in patients (pts) with advanced hepatocellular carcinoma (aHCC) and Child-Pugh B (CPB) status. Journal of Clinical Oncology. 2019; 37(4_suppl):327-327.
42. Brahmer J R, Lacchetti C, Schneider B J, Atkins M B, Brassil K J, Caterino J M, et al. Management of Immune-Related Adverse Events in Patients Treated With Immune Checkpoint Inhibitor Therapy: American Society of Clinical Oncology Clinical Practice Guideline. Journal of clinical oncology: official journal of the American Society of Clinical Oncology. 2018; 36(17):1714-1768.

What is claimed is:

1. A mixture of antibodies comprising:
(a) an anti-human Programmed Death 1 (anti-hPD1) antibody comprising a heavy chain (HC) and a light chain (LC), wherein (1) the HC of the anti-hPD1 antibody is encoded by a nucleic acid sequence which encodes the amino acid sequence of SEQ ID NO: 1, and (2) the LC of the anti-hPD1 antibody is encoded by a nucleic acid sequence which encodes the amino acid sequence of SEQ ID NO: 5; and
(b) an anti-human cytotoxic T-lymphoctye associated protein 4 (anti-hCTLA4) antibody comprising an HC and an LC, wherein (1) the HC of the anti-hCTLA4 antibody is encoded by a nucleic acid sequence which encodes the amino acid sequence of SEQ ID NO: 13, and (2) the LC of the anti-hCTLA4 antibody is encoded by a nucleic acid sequence which encodes the amino acid sequence of SEQ ID NO: 17;
wherein the weight for weight (w/w) ratio of the amount of the anti-hCTLA4 antibody in the mixture to the amount of the anti-hPD1 antibody in the mixture (anti-hCTLA4:anti-hPD1 ratio) ranges from 1:1 to 1:4,
wherein the anti-hPD1 antibody has an in vivo half-life (t_{1/2}) in a single dose study in cynomolgus monkeys of 220 to 380 hours and/or the anti-hPD1 antibody has an in vivo t_{1/2} of 135 to 300 hours when administered to a human who has not previously been dosed with the anti-hPD1 antibody, and
wherein the anti-hCTLA4 antibody has an in vivo t_{1/2} in a single dose study in cynomolgus monkeys of 40 to 150 hours and/or the anti-hCTLA4 antibody has an in vivo t_{1/2} of 90 to 210 hours when administered to a human who has not previously been dosed with the anti-hCTLA4 antibody.

2. The mixture of claim 1,
wherein the nucleic acid sequence which encodes the amino acid sequence of SEQ ID NO: 1 also encodes the amino acid sequence of SEQ ID NO: 10,
wherein the nucleic acid sequence which encodes the amino acid sequence of SEQ ID NO: 5 also encodes the amino acid sequence of SEQ ID NO: 12,
wherein the nucleic acid sequence which encodes the amino acid sequence of SEQ ID NO: 13 also encodes the amino acid sequence of SEQ ID NO: 22, and
wherein the nucleic acid sequence which encodes the amino acid sequence of SEQ ID NO: 17 also encodes the amino acid sequence of SEQ ID NO: 24.

3. The mixture of claim 1, wherein the anti-hCTLA4:anti-hPD1 ratio ranges from an amount selected from the group consisting of: from 1:1 to 1:3; from 1:1.2 to 1:2.5; from 1:1.5 to 1:2.5; and from 1:1.7 to 1:2.3.

4. The mixture of any one of claim 1, further comprising one or more of (a)-(e) as follows:
(a) wherein the amino acid sequences of the HC and LC of the anti-hPD1 antibody are encoded by the nucleic acid sequences of SEQ ID NOs: 2 and 6, respectively; and
the amino acid sequences of the HC and LC of the anti-hCTLA4 antibody are encoded by the nucleic acid sequences of SEQ ID NOs: 14 and 18, respectively;
(b) wherein the anti-hPD1 antibody has an in vivo t_{1/2} in a single dose study in cynomolgus monkeys of 250 to 350 hours and/or the anti-hPD1 antibody has an in vivo t_{1/2} of 140 to 250 hours when administered to a human who has not previously been dosed with the anti-hPD1 antibody, and
wherein the anti-hCTLA4 antibody has an in vivo t_{1/2} in a single dose study in cynomolgus monkeys of 70 to 130 hours and/or the anti-hCTLA4 antibody has an in vivo t_{1/2} of 90 to 140 hours when administered to a human who has not previously been dosed with the anti-hCTLA4 antibody;
(c) wherein when the mixture is administered to a group of at least ten human patients at a dose of no more than 5 mg/kg, no more than 15%, 14%, 13%, 12%, or 11% of the patients experience a grade 3 or grade 4 adverse event (AE);
(d) wherein when the mixture is administered to a group of at least ten human patients at a dose of no more than 5 mg/kg, no more than 10%, 9%, or 8% of the patients experience a grade 3 or grade 4 AE;
and/or
(e) wherein when the mixture is administered to a group of at least ten human patients at a dose of no more than 5 mg/kg, no more than 7%, 6%, or 5% of the patients experience a grade 3 or grade 4 AE.

5. A pharmaceutical composition comprising the mixture of claim 1.

6. The pharmaceutical composition of claim 5,
wherein the pH of the pharmaceutical composition is from pH 4.5 to pH 5.5; and/or
wherein the total protein concentration in the composition is from 20 mg/mL to 30 mg/mL; and/or
wherein the pharmaceutical composition has an osmolality from 250 to 380 mOsm/kg.

7. One or more polynucleotide(s) encoding the mixture of claim 1.

8. The polynucleotide(s) of claim 7, wherein the polynucleotide(s) comprise the nucleic acid sequences of SEQ ID NOs: 2, 6, 14, and 18.

9. One or more vector(s) comprising the polynucleotide(s) of claim 7, wherein the vector(s) is selected from one or more of: viral vector(s); oncolytic viral vector(s); retroviral, adenoviral, adeno-associated viral (AAV), vaccinia viral, modified vaccina viral Ankara (MVA), herpes viral, lentiviral, measles viral, coxsackie viral, Newcastle Disease viral, reoviral, and/or poxviral vector(s).

10. A host cell comprising the polynucleotide(s) of claim 16, wherein the host cell can produce a mixture of anti-hCTLA4:anti-hPD1 antibodies.

11. The host cell of claim 10, wherein the anti-hCTLA4:anti-hPD1 ratio of the mixture produced by the host cell ranges from an amount selected from the group consisting of: from 1:1.2 to 1:3; from 1:1.5 to 1:2.5; and from 1:1.7 to 1:2.3.

12. The host cell of claim 10, which is a CHO cell or a mouse myeloma cell.

13. A method for making a mixture of antibodies comprising the following steps:
culturing the host cell of claim 10; and
recovering the mixture of antibodies from the culture supernatant or the host cell mass.

14. A method for treating a patient having a cancer, an immunodeficiency disorder, or an infection comprising:
(a) administering to the patient a dose of the mixture of claim 1, or a pharmaceutical composition thereof, to the patient.

15. The method of claim 14(a), wherein the dose of the mixture or pharmaceutical composition is administered about twice a week, once a week, or once every two, three, four, five, six, seven, or eight weeks, and wherein the dose of the mixture or pharmaceutical composition is described by one or more of the following:
(1) the dose is at least about 0.5, 1.0, 2.0, 3.0, 4.0, 5.0, 6.0, 7.0, or 8.0 mg/kg;
(2) the dose is at most about 9, 8, 7, 6, 5, 4, or 3 mg/kg;
(3) the dose is about 1.0, 2.0, 3.0, 4.0, 5.0, 6.0, 7.0, or 8.0 mg/kg;
(4) the dose is about 200, 225, 250, 275, 300, 325, 350, 375, 400, 425, 450, 475, or 500 mg;
(5) the dose is at least about 75, 100, 125, 150, 200, 225, or 250 mg; and
(6) the dose is at most about 600, 500, 400, or 300 mg.

16. The method of claim 15, wherein
the dose is at least 3 mg/kg and no more than 5 mg/kg, and/or
the dose is at least 180 mg and no more than 400 mg,
wherein the dose is administered about once every three weeks.

17. The method of claim 16, wherein the dose is about 5 mg/kg.

18. The method of claim 16, wherein the dose is 300 to 400 mg.

19. The method of claim 15,
wherein the patient has a cancer,
wherein the mixture or the pharmaceutical composition is administered to at least 10 patients, and
wherein the objective response rate (ORR) is at least 5, 10, 15, 20, 25, 30, or 35 percent and/or the disease control rate (DCR) is at least 25, 30, 35, 40, 45, 50, 55, or 60 percent.

20. The method of any one of claim 14, further comprising one or more of the following:
wherein the dose of the mixture or pharmaceutical composition is administered by intravenous injection, including infusion or bolus injection, subcutaneous injection, or intramuscular injection; or
wherein the patient has melanoma, lung cancer, including squamous non-small cell lung cancer and small cell lung cancer, nasopharyngeal cancer, squamous cell carcinoma of the head and neck, gastric or gastroesophageal carcinoma, clear cell or non-clear cell renal cell carcinoma, urothelial cancer, soft tissue or bone sarcoma, mesothelioma, classical Hodgkin lymphoma, primary mediastinal large B-cell lymphoma, bladder cancer, Merkel cell carcinoma, neuroendocrine carcinoma, cervical cancer, hepatocellular carcinoma, ovarian cancer, or microsatellite instability high (MSI-H) or DNA mismatch repair deficient (dMMR) adult and pediatric solid tumors; or
wherein the patient is treated with a chemotherapeutic agent or radiation before, after, or concurrently with the dose of the mixture or the pharmaceutical composition; or
wherein the dose of the mixture or the pharmaceutical composition is administered to at least ten patients, wherein the patients to whom the dose has been administered are not treated concurrently with radiation or with a chemotherapeutic agent, and wherein no more than 15%, 14%, 13%, 12%, or 11% of the patients to whom the dose has been administered experience a grade 3 or grade 4 AE; or
wherein no more than 10%, 9%, or 8% of the patients to whom the dose has been administered experience a grade 3 or grade 4 AE; or
wherein no more than 7%, 6%, 5%, 4%, 3%, 2%, 1%, or 0% of the patients to whom the dose has been administered experience a grade 3 or grade 4 AE.

21. A method for treating a patient having a cancer, an immunodeficiency disorder, or an infection comprising:
(a) administering to the patient a dose of the polynucleotide(s) of claim 16 or a vector(s) comprising them.

22. The method of claim 21(a), wherein the dose of the polynucleotide(s) or the vector(s) is administered about twice a week, once a week, or once every two, three, four, five, six, seven, or eight weeks, and wherein the dose of the polynucleotide(s) or vector(s) is described by one or more of the following:
(1) the dose is at least about 5×10⁹ copies of the polynucleotide(s) or the vector(s) per kilogram of patient body weight (copies/kg);
(2) the dose is at most about 10¹⁵ copies/kg;
(3) the dose is from about 10¹⁰ copies/kg to about 10¹⁴ copies/kg; and
(4) the dose is about 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, or 10¹⁵ copies.

23. The method of any one of claim 11, further comprising one or more of the following:
wherein the dose of the polynucleotide(s), or vector(s) is administered by intravenous injection, including infusion or bolus injection, subcutaneous injection, or intramuscular injection; or
wherein the patient has melanoma, lung cancer, including squamous non-small cell lung cancer and small cell lung cancer, nasopharyngeal cancer, squamous cell carcinoma of the head and neck, gastric or gastroesophageal carcinoma, clear cell or non-clear cell renal cell carcinoma, urothelial cancer, soft tissue or bone sarcoma, mesothelioma, classical Hodgkin lymphoma, primary mediastinal large B-cell lymphoma, bladder cancer, Merkel cell carcinoma, neuroendocrine carcinoma, cervical cancer, hepatocellular carcinoma, ovarian cancer, or microsatellite instability high (MSI-H) or DNA mismatch repair deficient (dMMR) adult and pediatric solid tumors; or
wherein the patient is treated with a chemotherapeutic agent or radiation before, after, or concurrently with the dose of the mixture or the pharmaceutical composition; or
wherein the dose of the mixture or the pharmaceutical composition is administered to at least ten patients, wherein the patients to whom the dose has been administered are not treated concurrently with radiation or with a chemotherapeutic agent, and wherein no more than 15%, 14%, 13%, 12%, or 11% of the patients to whom the dose has been administered experience a grade 3 or grade 4 AE; or
wherein no more than 10%, 9%, or 8% of the patients to whom the dose has been administered experience a grade 3 or grade 4 AE; or
wherein no more than 7%, 6%, 5%, 4%, 3%, 2%, 1%, or 0% of the patients to whom the dose has been administered experience a grade 3 or grade 4 AE.


SEQUENCE LISTING (34 sequences)

SEQ ID NO: 1 - protein, 446 aa, Artificial Sequence
  Description of Artificial Sequence Synthetic polypeptide
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - DNA, 1344 bp, Artificial Sequence
  Description of Artificial Sequence Synthetic polynucleotide
caggtgcagctggtgcagtctggcgccgaagtgaagaaacctggcgcctccgtgaaggtgtcctgcaaggcttccggctacacctttaccaactactggatccactgggtgcgacaggcccctggacagggcctggaatggatgggcgagatcgacccctacgactcctacaccaactacaaccagaaattcaagggccgcgtgaccatgaccgtggacaagtccacctccaccgtgtacatggaactgtcctccctgcggagcgaggacaccgccgtgtactactgtgccagacccggcttcacctacggcggcatggatttttggggccagggcaccctcgtgaccgtgtcctctgcttctaccaagggcccctccgtgttccctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagcgggtggaatctaagtacggccctccctgccctccttgcccagcccctgagtttctgggcggacccagcgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgacatcgccgtggaatgggagtccaacggccagcctgagaacaactacaagaccaccccccctgtgctggactccgacggctccttcttcctgtactctcggctgacagtggataagtcccggtggcaggaaggcaacgtgttctcctgctccgtgatgcacgaggccctgcacaaccactacacccagaagtccctgtccctgtctctgggaaagtgataa

SEQ ID NO: 3 - protein, 119 aa, Artificial Sequence
  Description of Artificial Sequence Synthetic polypeptide
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 4 - DNA, 357 bp, Artificial Sequence
  Description of Artificial Sequence Synthetic polynucleotide
caggtgcagctggtgcagtctggcgccgaagtgaagaaacctggcgcctccgtgaaggtgtcctgcaaggcttccggctacacctttaccaactactggatccactgggtgcgacaggcccctggacagggcctggaatggatgggcgagatcgacccctacgactcctacaccaactacaaccagaaattcaagggccgcgtgaccatgaccgtggacaagtccacctccaccgtgtacatggaactgtcctccctgcggagcgaggacaccgccgtgtactactgtgccagacccggcttcacctacggcggcatggatttttggggccagggcaccctcgtgaccgtgtcctct

SEQ ID NO: 5 - protein, 220 aa, Artificial Sequence
  Description of Artificial Sequence Synthetic polypeptide
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 666 bp, Artificial Sequence
  Description of Artificial Sequence Synthetic polynucleotide
gacatccagatgacccagtccccctccagcctgtctgcctctgtgggcgacagagtgaccatcacatgcaagtcctcccagtccctgttcaactccggcaaccagaagaactacctggcctggtatcagcagaaacccggcaaggtgcccaagctgctgatctacggcgcctccaccagagactctggcgtgccctacagattctccggctctggctctggcaccgactttaccctgaccatcagctccctgcagcccgaggatgtggccacctactactgccagaacgaccactactacccctacaccttcggcggaggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgctgataa

SEQ ID NO: 7 - protein, 113 aa, Artificial Sequence
  Description of Artificial Sequence Synthetic polypeptide
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 8 - DNA, 339 bp, Artificial Sequence
  Description of Artificial Sequence Synthetic polynucleotide
gacatccagatgacccagtccccctccagcctgtctgcctctgtgggcgacagagtgaccatcacatgcaagtcctcccagtccctgttcaactccggcaaccagaagaactacctggcctggtatcagcagaaacccggcaaggtgcccaagctgctgatctacggcgcctccaccagagactctggcgtgccctacagattctccggctctggctctggcaccgactttaccctgaccatcagctccctgcagcccgaggatgtggccacctactactgccagaacgaccactactacccctacaccttcggcggaggcaccaaggtggaaatcaag

SEQ ID NO: 9 - protein, 119 aa, Artificial Sequence
  Description of Artificial Sequence Synthetic polypeptide
  MOD_RES (1)..(1): Pyroglutamate
EVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 10 - protein, 445 aa, Artificial Sequence
  Description of Artificial Sequence Synthetic polypeptide
  MOD_RES (1)..(1): Pyroglutamate
  MOD_RES (161)..(161): Asparagine or a deamidated version of asparagine
  MOD_RES (314)..(314): Asparagine or a deamidated version of asparagine
  MOD_RES (360)..(360): Asparagine or a deamidated version of asparagine
  MOD_RES (383)..(383): Asparagine or a deamidated version of asparagine
  MOD_RES (420)..(420): Asparagine or a deamidated version of asparagine
EVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 113 aa, Artificial Sequence; identical to SEQ ID NO: 7
  Description of Artificial Sequence Synthetic polypeptide
  MOD_RES (31)..(31): Asparagine or a deamidated version of asparagine
  MOD_RES (96)..(96): Asparagine or a deamidated version of asparagine

SEQ ID NO: 12 - protein, 220 aa, Artificial Sequence; identical to SEQ ID NO: 5
  Description of Artificial Sequence Synthetic polypeptide
  MOD_RES (31)..(31): Asparagine or a deamidated version of asparagine
  MOD_RES (96)..(96): Asparagine or a deamidated version of asparagine
  MOD_RES (144)..(144): Asparagine or a deamidated version of asparagine

SEQ ID NO: 13 - protein, 448 aa, Artificial Sequence
  Description of Artificial Sequence Synthetic polypeptide
QVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSSASTKGPSVFPLAPSSKSTSGGTAALGCLVDDYFPEPVTVSWNSGALTSGVHTCPACLQSSGLYSLSSVVTVPSSSLGTQTYICNVNHKPSNTKVDKKVEPKSGDKTHTCPPCPAPELLGGPSVFLFPPKPKDTLMISKTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 14 - DNA, 1350 bp, Artificial Sequence
  Description of Artificial Sequence Synthetic polynucleotide
caggtgcagctggtggaatctggcggcggagtggtggaaccaggcagaagcctgagactgagctgtgccgccagcggcttcaccttcagcagctacggaatgcactgggtgcgccaggcccctggcaaaggactggaatgggtggccgtgatctggtacaagcccagcgagaaggactacgccgacagcgccaagggccggttcaccatctcccgggacaacagcaagaacaccctgtacctgcagatgaacagcctgcgggccgaggacaccgccgtgtactattgtgctagaggcggcctgctgggctacttcgactattggggccagggcaccctcgtgaccgtgtctagcgctagcaccaagggcccatccgtcttccccctggcgccctcctccaagagcacctctgggggcacagcggccctgggctgcctggtcgacgactacttccccgaaccggtgacggtgtcgtggaactcaggcgccctgaccagcggcgtgcacacctgcccggcttgcctacagtcctcaggactctactccctcagcagcgtggtgaccgtgccctccagcagcttgggcacccagacctacatctgcaacgtgaatcacaagcccagcaacaccaaggtggacaagaaagttgagcccaaatctggcgacaaaactcacacatgcccaccgtgcccagcacctgaactcctggggggaccgtcagtcttcctcttccccccaaaacccaaggacaccctcatgatctccaagacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctgcggtccgacggctccttcttcctctatagcgagctcaccgtggacaagagcaggtggcagcaggggaacgtcttctcatgctccgtgatgcatgaggctctgcacaaccactacacgcagaaaagcctctccctgtctccgggtaaatgatga

SEQ ID NO: 15 - protein, 118 aa, Artificial Sequence
  Description of Artificial Sequence Synthetic polypeptide
QVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSS

SEQ ID NO: 16 - DNA, 354 bp, Artificial Sequence
  Description of Artificial Sequence Synthetic polynucleotide
caggtgcagctggtggaatctggcggcggagtggtggaaccaggcagaagcctgagactgagctgtgccgccagcggcttcaccttcagcagctacggaatgcactgggtgcgccaggcccctggcaaaggactggaatgggtggccgtgatctggtacaagcccagcgagaaggactacgccgacagcgccaagggccggttcaccatctcccgggacaacagcaagaacaccctgtacctgcagatgaacagcctgcgggccgaggacaccgccgtgtactattgtgctagaggcggcctgctgggctacttcgactattggggccagggcaccctcgtgaccgtgtctagc

SEQ ID NO: 17 - protein, 214 aa, Artificial Sequence
  Description of Artificial Sequence Synthetic polypeptide
EIVLTQSPGTLSLSPGERATLSCRASQSINSYLAWYQQKPGQAPRPLIYGVSSRATGIPDRFSGSGSGTDFTLTISRLEPEDFAVYYCQQYGRYPFTFGPGTKVDIKRTVAAPSVFIFPPSDEQLKSGTAKVVCLLNNFYPREAKVQWKVDNALQSGNSCECVTEQDSKDSTYSLSSTLTLSKADYEKHKVYACEVTHQGLSSPVTKSFNRGES

SEQ ID NO: 18 - DNA, 648 bp, Artificial Sequence
  Description of Artificial Sequence Synthetic polynucleotide
gagatcgtgctgacccagagccctggcaccctgtcactgtctccaggcgagagagccaccctgagctgtagagccagccagagcatcaacagctacctggcctggtatcagcagaagcccggacaggcccccagacccctgatctatggcgtgtccagcagagccaccggcatccccgatagattttccggcagcggctccggcaccgacttcaccctgacaatcagcagactggaacccgaggactttgccgtgtattactgccagcagtacggcagataccctttcaccttcggcccaggaacaaaagtggacatcaagcgtacggtggctgcaccatctgtcttcatcttcccgccatctgatgagcagttgaaatctggaactgccaaggttgtgtgcctgctgaataacttctatcccagagaggccaaagtacagtggaaggtggataacgccctccaatcgggtaactcctgcgagtgtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagagctgatga

SEQ ID NO: 19 - protein, 107 aa, Artificial Sequence
  Description of Artificial Sequence Synthetic polypeptide
EIVLTQSPGTLSLSPGERATLSCRASQSINSYLAWYQQKPGQAPRPLIYGVSSRATGIPDRFSGSGSGTDFTLTISRLEPEDFAVYYCQQYGRYPFTFGPGTKVDIK

SEQ ID NO: 20 - DNA, 321 bp, Artificial Sequence
  Description of Artificial Sequence Synthetic polynucleotide
gagatcgtgctgacccagagccctggcaccctgtcactgtctccaggcgagagagccaccctgagctgtagagccagccagagcatcaacagctacctggcctggtatcagcagaagcccggacaggcccccagacccctgatctatggcgtgtccagcagagccaccggcatccccgatagattttccggcagcggctccggcaccgacttcaccctgacaatcagcagactggaacccgaggactttgccgtgtattactgccagcagtacggcagataccctttcaccttcggcccaggaacaaaagtggacatcaag

SEQ ID NO: 21 - protein, 118 aa, Artificial Sequence
  Description of Artificial Sequence Synthetic polypeptide
  MOD_RES (1)..(1): Pyroglutamate
EVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSS

SEQ ID NO: 22 - protein, 447 aa, Artificial Sequence
  Description of Artificial Sequence Synthetic polypeptide
  MOD_RES (1)..(1): Pyroglutamate
  MOD_RES (316)..(316): Asparagine or a deamidated version of asparagine
  MOD_RES (362)..(362): Asparagine or a deamidated version of asparagine
  MOD_RES (385)..(385): Asparagine or a deamidated version of asparagine
EVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSSASTKGPSVFPLAPSSKSTSGGTAALGCLVDDYFPEPVTVSWNSGALTSGVHTCPACLQSSGLYSLSSVVTVPSSSLGTQTYICNVNHKPSNTKVDKKVEPKSGDKTHTCPPCPAPELLGGPSVFLFPPKPKDTLMISKTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPG

SEQ ID NO: 23 - protein, 107 aa, Artificial Sequence; identical to SEQ ID NO: 19
  Description of Artificial Sequence Synthetic polypeptide
  MOD_RES (30)..(30): Asparagine or a deamidated version of asparagine

SEQ ID NO: 24 - protein, 214 aa, Artificial Sequence; identical to SEQ ID NO: 17
  Description of Artificial Sequence Synthetic polypeptide
  MOD_RES (30)..(30): Asparagine or a deamidated version of asparagine
  MOD_RES (137)..(137): Asparagine or a deamidated version of asparagine

SEQ ID NO: 25 - protein, 12 aa, Artificial Sequence
  Description of Artificial Sequence Synthetic peptide
  MOD_RES (7)..(7): Asparagine or a deamidated version of asparagine
SSQSLFNSGNQK

SEQ ID NO: 26 - protein, 42 aa, Artificial Sequence
  Description of Artificial Sequence Synthetic polypeptide
  MOD_RES (29)..(29): Asparagine or a deamidated version of asparagine
FSGSGSGTDFTLTISSLQPEDVATYYCQNDHYYPYTFGGGTK

SEQ ID NO: 27 - protein, 16 aa, Artificial Sequence
  Description of Artificial Sequence Synthetic peptide
  MOD_RES (12)..(12): Asparagine or a deamidated version of asparagine
SGTASVVCLLNNFYPR

SEQ ID NO: 28 - protein, 49 aa, Artificial Sequence
  Description of Artificial Sequence Synthetic polypeptide
  MOD_RES (12)..(12): Asparagine or a deamidated version of asparagine
DYFPEPVTVSWNSGALTSGVHTFPAVLQSSGLYSLSSVVTVPSSSLGTK

SEQ ID NO: 29 - protein, 16 aa, Artificial Sequence
  Description of Artificial Sequence Synthetic peptide
  MOD_RES (14)..(14): Asparagine or a deamidated version of asparagine
VVSVLTVLHQDWLNGK

SEQ ID NO: 30 - protein, 10 aa, Artificial Sequence
  Description of Artificial Sequence Synthetic peptide
  MOD_RES (1)..(1): Asparagine or a deamidated version of asparagine
NQVSLTCLVK

SEQ ID NO: 31 - protein, 22 aa, Artificial Sequence
  Description of Artificial Sequence Synthetic peptide
  MOD_RES (14)..(14): Asparagine or a deamidated version of asparagine
GFYPSDIAVEWESNGQPENNYK

SEQ ID NO: 32 - protein, 23 aa, Artificial Sequence
  Description of Artificial Sequence Synthetic peptide
  MOD_RES (5)..(5): Asparagine or a deamidated version of asparagine
WQEGNVFSCSVMHEALHNHYTQK

SEQ ID NO: 33 - protein, 30 aa, Artificial Sequence
  Description of Artificial Sequence Synthetic polypeptide
  MOD_RES (6)..(6): Asparagine or a deamidated version of asparagine
ASQSINSYLAWYQQKPGQAPRPLIYGVSSR

SEQ ID NO: 34 - protein, 11 aa, Artificial Sequence
  Description of Artificial Sequence Synthetic peptide
  MOD_RES (6)..(6): Asparagine or a deamidated version of asparagine
VVCLLNNFYPR